(19) United States
(12) Patent Application Publication
(10) Pub. No.: US 2023/0049529 A1
(43) Pub. Date: Feb. 16, 2023

(54) MODIFIED DOUBLE STRANDED OLIGONUCLEOTIDE

(71) Applicants: Alnylam Pharmaceuticals, Inc., Cambridge, MA (US); THE SCRIPPS RESEARCH INSTITUTE, La Jolla, CA (US)

(72) Inventors: Alexander V. KEL'IN, Cambridge, MA (US); Alex R. NANNA, La Jolla, CA (US); Christopher RADER, La Jolla, MA (US); Christopher THEILE, Cambridge, MA (US); Justin PIERSON, Cambridge, MA (US); Kevin FITZGERALD, Cambridge, MA (US); Zhi Xiang VOO, Palm Beach Gardens, FL (US)

(73) Assignees: Alnylam Pharmaceuticals, Inc., Cambridge, MA (US); THE SCRIPPS RESEARCH INSTITUTE, La Jolla, CA (US)

(21) Appl. No.: 17/621,029

(22) PCT Filed: Jun. 18, 2020
(86) PCT No.: PCT/US2020/038475

§ 371 Date: Dec. 20, 2021

Related U.S. Application Data
(60) Provisional application No. 62/864,755, filed on Jun. 21, 2019.

(51) Int. Cl. A61K 47/68; C12N 15/113; A61K 47/54; C07K 16/28; C07K 16/32

(52) U.S. Cl. CPC A61K 47/6849; C12N 15/113; A61K 47/549; C07K 16/2803; C07K 16/2878; C07K 16/2896; A61K 47/6807; C07K 16/32; C12N 2310/3513; C12N 2310/322; C12N 2310/3231; C12N 2310/321; C12N 2310/315; C12N 2310/14; C07K 2317/31; C07K 2317/24; A61K 2039/505

(57) ABSTRACT

The disclosure is directed to dual variable domain immunoglobulin double-stranded RNA conjugates that are advantageous for inhibition of target gene expression, as well as compositions suitable for therapeutic use. The dual variable domain immunoglobulin comprises a first variable domain that binds to a binding target, and a second variable domain that comprises a reactive residue, where the linker is covalently conjugated to the reactive residue. The dsRNA is linked to the linker and is capable of inhibiting the expression of the target gene by RNA interference. The disclosure also provides pharmaceutical compositions comprising these conjugate and methods of inhibiting the expression of a target gene by administering these conjugates, e.g., for the treatment of various disease conditions.

### RELATED APPLICATIONS

This application is a 35 U.S.C. § 371 National Phase Entry Application of International Patent Application No. PCT/US2020/038475 filed on Jun. 18, 2020 which claims benefit under 35 U.S.C. § 119(e) of the U.S. Provisional Application No. 62/864,755 filed Jun. 21, 2019, the content of which is incorporated herein by reference in their entireties.

### SEQUENCE LISTING

The instant application contains a Sequence Listing which has been submitted electronically in ASCII format and is hereby incorporated by reference in its entirety. Said ASCII copy, created on Jul. 20, 2020, is named 051058-095570WOPT_SL.txt and is 76,163 bytes in size.

### FIELD OF THE INVENTION

The invention relates to dual variable domain immunoglobulin siRNA conjugates that are advantageous for inhibition of target gene expression, as well as compositions suitable for therapeutic use. Additionally, the invention provides methods of inhibiting the expression of a target gene by administering these conjugates, e.g., for the treatment of various diseases.

### BACKGROUND

RNA interference or “RNAi” is a term initially coined by Fire and co-workers to describe the observation that double-stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

The 1998 discovery of RNA mediated posttranscriptional gene silencing was pivotal in biological research. This process, known as RNA interference (RNAi), enabled the specific knockdown of any gene making it a commonly used technique in all biological research. From a therapeutic standpoint, RNAi has the advantage of being able to target any disease-associated RNA-based factor, many of which are considered “undruggable” by small molecules. Furthermore, the sequence-specific target recognition makes off-target toxicity less of a concern. There are several RNAi based strategies that use different classes of RNA for efficient knockdown. Short interfering RNAs (siRNAs) are one type that are fully complementary to the target sequence on the transcript and are introduced into target cells as a duplex. After entering cells, the siRNA is loaded into an RNA-induced silencing complex (RISC). During the loading process, the passenger (sense) strand is removed and the guide (antisense) strand remains within the RISC where it binds to its complementary site on the target mRNA. The bound mRNA can then be cleaved by the nuclease activity of RISC and then further degraded by cellular nucleases.¹

Although siRNAs are highly efficient at gene silencing, there are several challenges that have to be overcome to enable their use as therapeutics: 1.) siRNA size and high negative charge prevents passive uptake into cells; 2.) unmodified siRNAs exhibit both a short half-life in biological matrices due to rapid degradation by nucleases, and 3.) siRNA is potentially immunogenic. For certain tissues, like the eye and lung, some of these problems can be overcome by local administration of siRNA via intravitreal injection and inhalation, respectively. For siRNA delivery to the liver, tremendous progress over the past years has yielded several clinically validated delivery technologies, which have been shown to be safe and effective in humans. One system comprises multi-component lipid nanoparticles (LNPs) in which the siRNAs are encapsulated during most of their journey.^{2,3} The LNPs are designed to release their siRNA payload into the cytoplasm of hepatocytes, where they can engage with the RISC machinery.^{4Error! Bookmark not defined.,Error! Bookmark not defined.} Another well-established and clinically established approach utilizes a multivalent N-acetylgalactosamine (GalNAc) ligand covalently conjugated to the siRNA. The ligand is designed to bind with high affinity and specificity to the asialoglycoprotein receptor (ASGPR), a cell surface receptor expressed on hepatocytes.⁴ In addition to the utilization of GalNAc targeting ligands, the development of advanced siRNA chemistries has been critical for the success of this approach.⁵ These approaches for siRNA delivery to the liver have transformed the field and led to the first FDA approved RNAi based therapy in 2018 (Patisiran; Onpattro; Alnylam Pharmaceuticals) for the treatment of hereditary transthyretin amyloidosis (hATTR) with polyneuropathy.

Although there have been several advances in RNAi based therapies directed towards the liver, the ability to target other tissues is highly desirable. Monoclonal antibodies (mAbs) are particularity well suited as delivery vehicles because of their high specificity towards antigens expressed on target tissues and long-circulatory half-life.⁵ These properties have contributed towards mAbs being a highly successful therapeutic class with currently over 60 FDA approved antibody-based therapeutics.⁶ Furthermore, mAbs are an already validated delivery vehicle for the generation of antibody-drug conjugates (ADCs), which involve the conjugation of highly potent small molecules for their selective delivery to target cancer cells.⁷ Thus, the generation of antibody-RNA conjugates (ARCs) is a promising strategy for the delivery of siRNA to target cells. There have been several methods to prepare ARCs, but these strategies use nonspecific conjugation. See, for example,⁸⁻¹⁴ resulting in mixtures or site-specific methods that require multiple steps and the introduction of mutations.¹⁵⁻⁴⁷

### SUMMARY

The disclosure provides dual variable domain (DVD) immunoglobulin conjugates and uses thereof are provided. Generally, the conjugate comprises a DVD immunoglobulin molecule having a first and a second variable domain, and a dsRNA molecule that is covalently conjugated to the second variable domain via a linker. Methods of making and using the conjugates for inhibiting target gene expression and therapeutic uses are also provided.

Generally, a conjugate of the invention comprises a dual variable domain immunoglobulin molecule (Ig) or an antigen-binding fragment thereof, and a double-stranded RNA (dsRNA) molecule, linked together via a linker. Usually, the dual variable domain immunoglobulin molecule comprises. (i) a first variable domain that binds to a binding target; and (ii) a second variable domain that comprises a reactive residue, where the linker is covalently conjugated to the reactive residue.

The conjugates disclosed herein can be represented by the formula: Ig-(L-R)ₙ, where Ig is a dual variable domain immunoglobulin molecule, or an immunoglobulin-fragment (antigen-binding fragment) thereof, where the dual variable domain immunoglobulin molecule comprises a first variable domain that binds to a binding target, and a second variable domain that comprises a reactive residue; L is a linker that is covalently conjugated to the reactive residue of the second variable domain of Ig; R is a double-stranded RNA molecule, and n is an integer selected from the group consisting of 1, 2, 3, 4, 5, 6, 7, 8, 9, 10, 11, and 12. In an aspect, a reactive residue allows for the stoichiometric attachment of L, and encompasses, but is not limited to, natural and unnatural amino acids containing SH, NH2, OH, SeH, N3, alkyne, alkene, strained alkynes, strained alkenes, C═O and activated C—H as reactive functional groups. In some embodiments of the various aspects disclosed herein, the reactive residue of the second variable domain is lysine or arginine.

The double-stranded RNA molecule is conjugated to the linker and comprises a sense strand and an antisense strand, each strand having 14 to 40 nucleotides, wherein the antisense strand has sufficient complementarity to a target sequence to mediate RNA interference, and the dsRNA is capable of inhibiting the expression of the target gene. In some embodiments of the various aspects disclosed herein, the dsRNA molecule has at least one, e.g., two, three, four, five, six, seven, eight, nine or all ten of the following characteristics: (i) a melting temperature (Tm) of from about 40° C. to about 80° C.; (ii) the antisense strand comprises 2, 3, 4, 5 or 6 2′-fluoro modifications; (iii) the antisense strand comprises 1, 2, 3 or 4 phosphorothioate internucleotide linkages; (iv) the sense strand is conjugated with the linker; (v) the sense strand comprises 2, 3, 4 or 5 2′-fluoro modifications; (vi) the sense strand comprises 1, 2, 3 or 4 phosphorothioate internucleotide linkages; (vii) the dsRNA comprises at least four 2′-fluoro modifications; (viii) the dsRNA comprises a duplex region of 12-40 nucleotide pairs in length; (ix) the dsRNA has a blunt end at 5′end of the antisense strand; and (x) the dsRNA has an overhang at 3′-end of the antisense strand.

In another aspect, the disclosure provides a method for inhibiting the expression of a target gene sequence. Generally, the method comprises administering a conjugate described herein to a cell in an amount sufficient to inhibit expression of the target gene. The cell can be in vitro or in vivo.

In still another aspect, the disclosure provides a pharmaceutical composition comprising a conjugate described herein.

In yet another aspect, the disclosure provides a method for treating a subject using a conjugate described herein. Generally, the method for treatment comprises administering a therapeutically effective amount of a conjugate described herein to a subject in need thereof.

### BRIEF DESCRIPTION OF THE DRAWINGS

FIG. 1A is a schematic representation showing a comparison of h38C2 IgG1 to an anti-multiple myeloma (MM) DVD-IgG1. The DVD-IgG1 is composed of variable domains of anti-CD138, BCMA, or SLAMF7 (blue), h38C2 (green) with reactive lysine (Lys; K, yellow circle), and constant domains (gray). A fully human spacer sequence (ASTKGP, red lines) was used to join the two variable domains together.

FIG. 1B is a Coomassie stained SDS-PAGE confirming the purity of all MM-targeting DVD-IgG1s under non-reducing (expected ˜200 kDa) and reducing conditions (expected heavy chain ˜63 kDa, light chain ˜36 KDa). Molecular weights from a pre-stained protein ladder are shown on the left.

FIG. 1C is a flow cytometry analysis showing specific binding of DVD-IgG1s against three MM cell lines (U-266, NCI-H929, and RPMI-8226). h38C2 IgG1 (black) was used as a negative control.

FIG. 2 shows structures of siRNA compounds. β-lactam hapten (blue) functionalized siRNA at the 3′ (4, SEQ ID NO: 40 and 43) and 5′ (5, SEQ ID NO: 39 and 43) end targeting CTNNB1 for DVD-IgG1 attachment. Control β-lactam hapten functionalized siRNA at the 3′ end (6, SEQ ID NO: 41 and 44) targeting human transthyretin (TTR^{Error! Bookmark not defined.}), which is an irrelevant target in this study. Control siRNA targeting CTNNB1 lacking the β-lactam hapten moiety (7, SEQ ID NO: 37 and 43) for DVD-IgG1 conjugation. Black circles in the siRNA indicate 2′-OMe-modified nucleosides, green circles stand for 2′-F-modified nucleosides, and blue circles for 2′-NMA 5-Me-U nucleosides containing a 5′-vinylphosphonate (VP) moiety. Yellow bars denote phosphorothioate (PS) linkages for exonuclease protection. The combination of 2′-OMe and 2′-F-modified nucleosides with PS linkages is in accordance with the previously reported “enhanced stabilization chemistry” (ESC) template.^{Error! Bookmark not defined.}

FIG. 3A is a schematic representation of ARC assembly. ARCs (8-13) were assembled by incubating DVD-IgG1s (1-3) with ten equivalents (eq) of β-lactam siRNA (4 and 5) at room temperature (rt) for 2 h. siRNA attachment (red and black helix) occurs at the reactive Lys of h38C2 (K, yellow circle), located in the inner heavy chain variable domain of the DVDs, to form a stable amide bond.

FIG. 3B is a line graph showing the catalytic retro-aldol activity of the reactive Lys of h38C2. The activity was measured using methodol as a substrate, which is converted to a fluorescent aldehyde and detected. The signal is reported in relative fluorescent units (RFU; mean f SD of triplicates). The assembled ARCs (8-13) were catalytically inactive due to amide formation at the reactive Lys, indicating complete conjugation. Unconjugated DVD-IgG1s (1-3) were used as positive controls and trastuzumab IgG1 (black) as a negative control.

FIG. 4A is a bar graph showing CTNNB1 mRNA knockdown in NCI-H929 cells after treatment with SLAMF7 (8 and 11), BCMA (9 and 12), or CD138 (10 and 13) targeting ARCs for 72 h at 37° C. at 90 nM (antibody concentration). Unconjugated DVD-IgG1s (1-3) (black) and transfected free siRNA (FIG. 2, 7) (white) were used as negative and positive controls, respectively.

FIG. 4B is a bar graph showing dose response of BCMA-targeting ARCs (9 and 12) with NCI-H929 cells. BCMA ARC (14) is conjugated to an siRNA targeting human TTR (FIG. 2, 6) and was used as a negative control. Error bars in (A) and (B) correspond to biological duplicates. A student's t-test was used to determine significance when each group was compared to the untreated group.

FIG. 5 is a bar graph showing CTNNB1 mRNA knockdown in NCI-H929 cells after treatment with unpurified or purified BCMA targeting ARCs (9 and 12) for 72 h at 37° C. at 90 nM (antibody concentration). Unconjugated anti-BCMA DVD-IgG1 (2), anti-BCMA ARCs conjugated to an siRNA targeting human TTR (14), and anti-HER2 ARCs (15 and 16) conjugated to an siRNA targeting CTNNB1 were used as negative controls. Transfected free siRNA (FIG. 2, 7) was used as a positive control. Error bars correspond to biological triplicates.

FIG. 6 is gel picture showing CTNNB1 protein knockdown in NCI-H929 cells after treatment with BCMA targeting ARCs (9 and 12, lanes 3 and 4) for 72 h at 37° C. at 90 nM (antibody concentration). Untreated cells (lane 1), unconjugated anti-BCMA DVD-IgG1 (2, lane 2), anti-BCMA ARC targeting TTR (14, lane 5), and anti-HER2 ARCs targeting CTNNB1 (15 and 16, lanes 6 and 7) were used as negative controls. Transfected free siRNA (7, lane 8) was used as a positive control. The western blot depicted is a representative example from 3 biological replicates.

FIG. 7 is a schematic representation showing the synthesis of an exemplary sense strand 19 (SEQ ID NO: 39) containing a β-lactam moiety by reaction of bis-β-lactam derivative 15 with the corresponding single-stranded siRNA 16 (SEQ ID NO: 36) containing an amino function appended at the 5′-end. Compounds 20 and 21 containing a β-lactam moiety at the 3′-end of the strand were prepared analogously from the corresponding precursor strands 17 and 18.

FIGS. 8A and 8B are HPLC chromatograms of 19 (SEQ ID NO: 18) without (FIG. 8A) and with butylamine (FIG. 8B) pretreatment. Complete cleavage of the product by HPLC eluents was observed. After pretreatment of an aliquot with excess of butylamine, the product was converted to the butylamine adduct prior to the chromatography.

FIG. 9 shows the catalytic retro-aldol activity of the reactive Lys of h38C2. The activity was measured using methodol as a substrate, which is converted to a fluorescent aldehyde and detected. The signal is reported in relative fluorescent units (RFU; mean f SD of triplicates). The anti-BCMA DVD-IgG1 (2) was conjugated to an siRNA-targeting human TTR (FIG. 2,6) using the conditions shown in FIG. 3A to generate the antibody-RNA conjugate (14). The ARC is catalytically inactive due to amide formation at the reactive Lys, indicating complete conjugation. Unconjugated anti-BCMA DVD-IgG1 (2) was used as a positive control and trastuzumab IgG1 (black) as a negative control.

FIG. 10A is a schematic showing optimized assembly of anti-BCMA ARC 9 and 12. ARCs were assembled by incubating the anti-BCMA DVD-IgG1 (2) with two equivalents (eq) of β-lactam siRNA (4 and 5; FIG. 2) at room temperature (rt) for 4 h. siRNA attachment (red and black helix) occurs at the reactive Lys of h38C2 (K, yellow circle), located in the inner heavy chain variable domain of the DVD-IgG1, to form a stable amide bond.

FIG. 10B show the catalytic retro-aldol activity of the reactive Lys of h38C2 of the anti-BCMA ARCs 9 and 12 (FIG. 10A). The activity was measured using methodol as a substrate, which is converted to a fluorescent aldehyde and detected. The signal is reported in relative fluorescent units (RFU; mean f SD of triplicates). The assembled ARCs (9 and 12) were catalytically inactive due to amide formation at the reactive Lys, indicating complete conjugation. Unconjugated anti-BCMA DVD-IgG1 (2) was used as a positive control and trastuzumab IgG1 (black) as a negative control.

FIGS. 11A-11D are size exclusion chromatographs showing purified anti-BCMA DVD-IgG1 2 (FIG. 11A) and ARCs anti-BCMA ARC 9 (FIG. 11B), anti-BCMA ARC 12 (FIG. 11C), and anti-BCMA ARC 14 (FIG. 11D). The major peaks are indicated in mL.

FIGS. 12A-12D show the assembly of the anti-HER2 control ARCs and their size-exclusion chromatographs. FIG. 12A shows the catalytic retro-aldol activity of the reactive Lys of h38C2. The activity was measured using methodol as a substrate after conjugation of the anti-HER2 DVD-IgG1 with β-lactam siRNA (4 and 5) as described in FIG. 3. The signal is reported in relative fluorescent units (RFU; mean f SD of triplicates). The assembled ARCs (15 and 16) were catalytically inactive due to amide formation at the reactive Lys, indicating complete conjugation. Unconjugated anti-HER2 DVD (blue) was used as a positive control and trastuzumab IgG1 (black) as a negative control. (B) anti-HER2 DVD. (C) anti-HER2 ARC (15). (D) anti-HER2 ARC (16). FIGS. 12-B-12D are size exclusion chromatographs showing purified anti-HER2 DVD (FIG. 12B), anti-HER2 ARC 15 (FIG. 12C), and anti-HER2 ARC 16 (FIG. 12D). The major peaks are indicated in mL.

FIGS. 13A and 13B are bar graphs showing β-catenin knockdown in U266 (FIG. 13A) and RPMI-8226 (FIG. 13B) cells after treatment with SLAMF7, BCMA, or CD138 targeting ARCs (8-13) for 72 h at 37° C. at 90 nM. Unconjugated DVDs (1-3) (black) and transfected free siRNA (6) (gray) were used as controls. A student's t-test was used to determine significance when each group was compared to the untreated group. The error bars correspond to biological triplicates

FIGS. 14A-14C show cytotoxicity of ARCs (top-to-down: 1, 8, 11, 2, 9, 12, 3, 10, 13, and 25, CD138 ADC is 28) following incubation with MM cell lines U266 (FIG. 14A), NCI-H929 (FIG. 14B) and RPMI-8226 (FIG. 14C) for 72 h at 37° C. (mean f SD of triplicates). Unconjugated DVDs were used as negative controls and an anti-CD138 DVD conjugated to cytotoxic MMAF (22, black) was used as a positive control.

FIGS. 15A-15C show surface plasmon resonance (SPR) binding analysis of exemplary anti-BSMA Fab-DVD conjugated with siRNA_4 (FIG. 15A) and with siRNA_5 (FIG. 15B) and without conjugation with an siRNA (FIG. 15C). The calculated equilibrium dissociation constants (K_{d}) were identical before and after conjugation, indicating that the conjugation with siRNA does not affect outer variable domain binding to BCMA.

FIG. 16 is a schematic representation of a DVD-IgG1 ARC construct (1:2 conjugation).

FIG. 17 is a schematic representation of an exemplary DVD-IgG1 ARC/EEP construct (1:2 conjugation) with a fusogenic peptide.

FIGS. 18 and 19 show size exclusion chromatography and flow cytometry analysis (FIG. 18) and Fab format, binding and surface plasmon resonance analysis (FIG. 19) of an exemplary DVD-IgG1 ARC (BCMA/CTNNB 1-A).

FIGS. 20 and 21 show knockdown of CTNNB1 mRNA (FIG. 20) and CTNNB1 protein (FIG. 21) with an exemplary DVD-IgG1 ARC (BCMA/CTNNB 1-A). Sequences in FIG. 20 are SEQ ID NO: 39 (top) and SEQ ID NO: 43 (bottom).

FIG. 22 shows pharmacokinetics of an exemplary DVD-IgG1 ARC (BCMA/CTNNB 1-A). Sequences are SEQ ID NO: 51 (top) and SEQ ID NO: 43 (bottom).

FIG. 23 is a schematic showing effect of interferon regulatory factor 4 (IRF4) in multiple myeloma. Adapted from Shaffer et al. Nature (2008), 454(7201):226-31 and Shaffer et al. Clin Cancer Res (2009), 15(9): 2954-2961.

FIG. 24 shows knockdown of IRF4 mRNA with an exemplary DVD-IgG1 ARC (BCMA/CTNNB 1-A). Sequences are SEQ ID NO: 52 (top) and SEQ ID NO: 53 (bottom).

FIG. 25 shows cytotoxicity of exemplary ARCs, with and without endosomal escape peptide (BCMA/IRF4 and SLAMF7/IRF4 (4-A=1:2) conjugates).

### DETAILED DESCRIPTION

While the preparation of structurally defined conjugates of dual variable domain (DVD) antibodies containing uniquely reactive lysine residues site-specifically conjugated to small molecule cargoes such as cytotoxic agents using a hapten-like β-lactam moiety as an attachment anchor has been described in the art (See, Nanna et al., Nat. Commun. 2017; and Int. Patent Publication No. WO2017/049139), the practical preparation of the corresponding DVD-siRNA conjugates for therapeutic use represented significant challenges with many unknowns. For example, large, positively charged siRNA cargo molecules can compromise binding affinity and cell internalization once conjugated to the DVD; the siRNA molecules can lose their gene-silencing activity once conjugated to the DVD; siRNA can have enhanced nucleolytic degradation once conjugated to the DVD; chemistry for conjugating drugs to DVD (e.g., labile β-lactams) is not compatible with the currently established solid support synthesis of siRNAs; the reactivity of β-lactam moiety attached to the siRNA may not be high enough to support efficient conjugation to the uniquely reactive Lys residue within the hydrophobic pocket of the DVD using mild and neutral conditions so to not compromise integrity of the DVD and the siRNA; and purification of DVD-siRNA conjugates is not obvious. Further, the attachment point on the siRNA can also comprise the activity of the siRNA.

The inventors have now discovered that conjugation of two siRNA molecules to DVD surprisingly and unexpectedly did not compromise the binding affinity and internalization of the DVD-Ig/dsRNA conjugates described herein. Further, they found surprisingly and unexpectedly that DVD conjugation at the 3′ or 5′-positions of a sense strand of siRNA does not compromise gene-silencing activity. Moreover, novel and nonobvious non-cleavable and cleavable bis-β-lactam linkers described herein can be used for post-conjugation to an siRNA containing alkyl-amino group after solid-support synthesis. In addition, siRNAs containing a 3-aryl-β-lactam moiety can be efficiently and quantitatively conjugated to the corresponding DVDs using mild and neutral conditions in aqueous buffer (phosphate buffered saline, pH 7.4) without compromising the integrity of either siRNA or DVD. It was also surprising and unexpected that DVD-siRNA conjugates can be efficiently purified using size-exclusion chromatography.

In one aspect, the invention provides a conjugate comprising: (i) a dual variable domain immunoglobulin molecule (Ig), or an antigen-binding fragment thereof, wherein the dual variable domain immunoglobulin molecule comprises a first variable domain that binds to a binding target and a second variable domain that comprises a reactive residue; (ii) a linker covalently conjugated to the reactive residue of the second variable domain of Ig; and (iii) a double-stranded RNA (dsRNA) molecule conjugated to the linker, where the dsRNA is capable of inhibiting the expression of a target gene, where the dsRNA comprises a sense strand and an antisense strand, each strand having 14 to 40 nucleotides, wherein the antisense strand has sufficient complementarity to the target sequence to mediate RNA interference.

dsRNA Molecules

Aspects of the invention include double-stranded RNA molecules. Generally, the dsRNA molecule comprises a sense strand (also referred to as passenger strand) and an antisense strand (also referred to as guide strand). Each strand of the dsRNA molecule independently can range from 12-40 nucleotides in length. For example, each strand independently can be between 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. In some embodiments, the antisense strand is longer, e.g., by 1, 2, 3, 4, or 5 nucleotides than the sense strand.

In some embodiments, the antisense strand is of length 18 to 35 nucleotides. In some embodiments, the antisense strand is 21-25, 19-25, 19-21 or 21-23 nucleotides in length. In some particular embodiments, the antisense strand is 23 nucleotides in length.

Similar to the antisense strand, the sense strand can be, in some embodiments, 18-35 nucleotides in length. In some embodiments, the sense strand is 21-25, 19-25, 19-21 or 21-23 nucleotides in length. In some particular embodiments, the antisense strand is 21 nucleotides in length.

In some particular embodiments, sense strand is 21 nucleotides in length and the antisense strand is 23 nucleotides in length.

The double-stranded RNA molecule has a double-stranded or duplex region. Generally, the duplex region (double-stranded region) is 12-40 nucleotide base pairs in length. For example, the dsRNA has a duplex region of 12-25 nucleotide pairs in length. In some embodiments, the dsRNA has a duplex region of 18, 19, 20, 21, 22, 22, 23, 24, or 25 nucleotide base pairs in length. In some particular embodiments, the dsRNA has a duplex region of 19, 20, 21 or 22 nucleotide base pairs in length.

The dsRNA molecule can comprise thermally stabilizing modifications. For example, the dsRNA molecule can comprise at least four, e.g., five, six, seven, eight, nine, ten, eleven, twelve, thirteen, fourteen, fifteen or more thermally stabilizing modifications.

Without limitations, the thermally stabilizing modifications all can be present in one strand. In some embodiments, both the sense and the antisense strands comprise at least one, e.g., two, three, four or more thermally stabilizing modifications. The thermally stabilizing modification can occur on any nucleotide of the sense strand or antisense strand. For instance, the thermally stabilizing modification can occur on every nucleotide on the sense strand and/or antisense strand; each thermally stabilizing modification can occur in an alternating pattern on the sense strand or antisense strand; or the sense strand and antisense strand both comprise thermally stabilizing modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the thermally stabilizing modifications on the antisense strand.

The antisense strand of the dsRNA molecule can comprise at least one, e.g., two, three, four, five, six, seven, eight, nine, ten or more thermally stabilizing modifications. In some embodiments, the antisense strand comprises two, three, four, five or six thermally stabilizing modifications. Without limitations, a thermally stabilizing modification in the antisense strand can be present at any position. In some embodiments, the antisense strand comprises at least three thermally stabilizing modifications. For example, the antisense strand comprises thermally stabilizing modifications at least at positions 2, 14 and 16 from the 5′-end. In some other embodiments, the antisense comprises at least four thermally stabilizing modifications. For example, the antisense comprises thermally stabilizing modifications at least at positions 2, 6, 14 and 16 from the 5′-end. In some further embodiments, the antisense strand comprises at least five thermally stabilizing modifications. For example, the antisense strand comprises thermally stabilizing modifications at least at positions 2, 6, 9, 14 and 16 from the 5′-end. In still some further embodiments, the antisense strand comprises at least six thermally stabilizing modifications. For example, the antisense strand comprises thermally stabilizing modifications at least at positions 2, 6, 8, 9, 14 and 16 from the 5′-end.

The sense strand of the dsRNA molecule can comprise at least one, e.g., two, three, four, five, six, seven, eight, nine, ten or more thermally stabilizing modifications. In some embodiments, the sense strand comprises two, three, four, or five thermally stabilizing modifications. For example, the sense strand comprises three or four thermally stabilizing modifications. Without limitations, a thermally stabilizing modification in the sense strand can be present at any positions. In some embodiments, the sense strand comprises at least three thermally stabilizing modifications. For example, the sense comprises thermally stabilizing modification at least at positions 7, 10 and 11 from the 5′-end. In some other embodiments, the sense strand comprises at least four thermally stabilizing modifications. For example, the sense comprises thermally stabilizing modification at least at positions 7, 9, 10 and 11 from the 5′-end.

In some embodiments, the sense strand comprises thermally stabilizing modifications at positions opposite or complimentary to positions 11, 12 and 15 of the antisense strand, counting from the 5′-end of the antisense strand. In some other embodiments, the sense strand comprises thermally stabilizing modifications at positions opposite or complimentary to positions 11, 12, 13 and 15 of the antisense strand, counting from the 5′-end of the antisense strand. In some embodiments, the sense strand comprises a block of two, three or four thermally stabilizing modification.

In some embodiments, the sense strand comprises thermally stabilizing modifications at least at positions 7, 9, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, and 11 from the 5′-end, and the antisense strand comprises thermally stabilizing modifications at least at positions 2, 6, 8, 9, 14 and 16 from the 5′-end.

In some embodiments, the sense strand comprises thermally stabilizing modifications at least at positions 7, 9, 10, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, 10, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, 10, and 11 from the 5′-end, and the antisense strand comprises thermally stabilizing modifications at least at positions 2, 6, 8, 9, 14 and 16 from the 5′-end.

In some embodiments, the sense strand does not comprise a thermally stabilizing modification in position opposite or complimentary to the thermally destabilizing modification of the duplex in the antisense strand.

Exemplary thermally stabilizing modifications include, but are not limited to 2′-fluoro modifications and locked nucleic acid (LNA).

The dsRNA molecule can comprise 2′-fluoro nucleotides, i.e., 2′-fluoro modifications. For example, the dsRNA molecule can comprise at least four, e.g., five, six, seven, eight, nine, ten, eleven, twelve, thirteen, fourteen, fifteen or more 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand and antisense strand both comprise 2′-fluoro modifications in an alternating pattern. The alternating pattern of the 2′-fluoro modifications on the sense strand can be the same or different from the antisense strand, and the alternating pattern of the 2′-fluoro modifications on the sense strand can have a shift relative to the alternating pattern of the 2′-fluoro modifications on the antisense strand.

The antisense strand of the dsRNA molecule can comprise at least two (e.g., two, three, four, five, six, seven, eight, nine, ten or more) 2′-fluoro nucleotides. In some embodiments, the antisense strand comprises two, three, four, five or six 2′-fluoro nucleotides. Without limitations, a 2′-fluoro modification in the antisense strand can be present at any position. In some embodiments, the antisense strand comprises at least three 2′-fluoro nucleotides. For example, the antisense strand comprises 2′-fluoro nucleotides at least at positions 2, 14 and 16 from the 5′-end. In some other embodiments, the antisense comprises at least four 2′-fluoro nucleotides. For example, the antisense comprises 2′-fluoro nucleotides at least at positions 2, 6, 14 and 16 from the 5′-end. In some further embodiments, the antisense strand comprises at least five 2′-fluoro nucleotides. For example, the antisense strand comprises 2′-fluoro nucleotides at least at positions 2, 6, 9, 14 and 16 from the 5′-end. In still some further embodiments, the antisense strand comprises at least six 2′-fluoro nucleotides. For example, the antisense strand comprises 2′-fluoro nucleotides at least at positions 2, 6, 8, 9, 14 and 16 from the 5′-end.

The sense strand of the dsRNA molecule can comprise at least two (e.g., two, three, four, five, six, seven, eight, nine, ten or more) 2′-fluoro nucleotides. In some embodiments, the sense strand comprises two, three, four, or five 2′-fluoro nucleotides. For example, the sense strand comprises three or four 2′-fluoro nucleotides. Without limitations, a 2′-fluoro modification in the sense strand can be present at any positions. In some embodiments, the sense strand comprises at least three 2′-fluoro nucleotides. For example, the sense comprises 2′-fluoro nucleotides at least at positions 7, 10 and 11 from the 5′-end. In some other embodiments, the sense strand comprises at least four 2′-fluoro nucleotides. For example, the sense comprises 2′-fluoro nucleotides at least at positions 7, 9, 10 and 11 from the 5′-end.

In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand comprises 2′-fluoro nucleotides at least at positions 7, 9, and 11 from the 5′-end, and the antisense strand comprises 2′-fluoro nucleotides at least at positions 2, 14 and 16 from the 5′-end. In some other embodiments, the sense strand comprises 2′-fluoro nucleotides at least at positions 7, 9, and 11 from the 5′-end, and the antisense strand comprises 2′-fluoro nucleotides at least at positions 2, 6, 9, 14 and 16 from the 5′-end. In yet some other embodiments, the sense strand comprises 2′-fluoro nucleotides at least at positions 7, 9, and 11 from the 5′-end, and the antisense strand comprises 2′-fluoro nucleotides at least at positions 2, 6, 8, 9, 14 and 16 from the 5′-end.

In some embodiments, the sense strand comprises 2′-fluoro nucleotides at least at positions 7, 9, 10, and 11 from the 5′-end, and the antisense strand comprises 2′-fluoro nucleotides at least at positions 2, 14 and 16 from the 5′-end. In some other embodiments, the sense strand comprises 2′-fluoro nucleotides at least at positions 7, 9, 10, and 11 from the 5′-end, and the antisense strand comprises 2′-fluoro nucleotides at least at positions 2, 6, 9, 14 and 16 from the 5′-end. In yet some other embodiments, the sense strand comprises 2′-fluoro nucleotides at least at positions 7, 9, 10, and 11 from the 5′-end, and the antisense strand comprises 2′-fluoro nucleotides at least at positions 2, 6, 8, 9, 14 and 16 from the 5′-end.

In some embodiments, the antisense strand does not comprise a 2′-fluoro nucleotide at positions 3-9, counting from 5′-end.

The dsRNA molecule can comprise at least one, e.g., one, two, three, four, five, six, seven, eight, nine, ten, eleven, twelve, thirteen, fourteen, fifteen, sixteen, seventeen, eighteen, nineteen, twenty or more 2′-OMe nucleotides. Without limitations, the 2′-OMe nucleotides all can be present in one strand. In some embodiments, both the sense and the antisense strands comprise at least one 2′-OMe nucleotide. The 2′-OMe modification can occur on any nucleotide of the sense strand or antisense strand. For instance, the 2′-OMe modification can occur on every nucleotide on the sense strand and/or antisense strand; each thermally stabilizing modification can occur in an alternating pattern on the sense strand or antisense strand; or the sense strand and antisense strand both comprise 2′-OMe modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the 2′-OMe modifications on the antisense strand.

The antisense strand of the dsRNA molecule can comprise at least one, e.g., two, three, four, five, six, seven, eight, nine, ten, eleven, twelve, thirteen, fourteen, fifteen, sixteen, seventeen or more 2′-OMe modifications. Without limitations, a thermally stabilizing modification in the antisense strand can be present at any position. In some embodiments, the antisense strand comprises at least three thermally stabilizing modifications.

For example, the antisense strand does not comprise 2′-OMe modifications at least at positions 2, 14 and 16 from the 5′-end. In some other embodiments, the antisense does not comprise 2′-OMe modifications at least at positions 2, 6, 14 and 16 from the 5′-end. In some further embodiments, the antisense strand does not comprise 2′-OMe modifications at least at positions 2, 6, 9, 14 and 16 from the 5′-end. In still some further embodiments, the antisense strand does not comprise 2′-OMe modifications at least at positions 2, 6, 8, 9, 14 and 16 from the 5′-end.

The sense strand of the dsRNA molecule can comprise at least one, e.g., two, three, four, five, six, seven, eight, nine, ten, eleven, twelve, thirteen, fourteen, fifteen, sixteen or more 2′-OMe modifications. Without limitations, a 2′-OMe modification in the sense strand can be present at any positions. In some embodiments, the sense does not comprise 2′-OMe modifications at least at positions 7, 10 and 11 from the 5′-end. In some other embodiments, the sense does not comprise 2′-OMe modifications at least at positions 7, 9, 10 and 11 from the 5′-end.

The dsRNA molecule can comprise locked nucleic acid (LNA). For example, the dsRNA molecule can comprise can comprise at least one, e.g., one, two, three, four, five, six, seven, eight, nine, ten, eleven, twelve, thirteen, fourteen, fifteen, sixteen, seventeen, eighteen, nineteen, twenty or more LNA modifications. Without limitations, the LNA nucleotides all can be present in one strand. In some embodiments, both the sense and the antisense strands comprise at least LNA modifications. The LNA modification can occur on any nucleotide of the sense strand or antisense strand. For instance, the LNA modification can occur on every nucleotide on the sense strand and/or antisense strand; each LNA modification can occur in an alternating pattern on the sense strand or antisense strand; or the sense strand and antisense strand both comprise LNA modifications in an alternating pattern. The alternating pattern of the LNA modifications on the sense strand can be the same or different from the antisense strand, and the alternating pattern of the LNA modifications on the sense strand can have a shift relative to the alternating pattern of the 2′-fluoro modifications on the antisense strand.

The antisense strand of the dsRNA molecule can comprise at least one, e.g., two, three, four, five, six, seven, eight, nine, ten or more LNA modifications. Without limitations, a LNA modification in the antisense strand can be present at any position.

The sense strand of the dsRNA molecule can comprise at least one, e.g., two, three, four, five, six, seven, eight, nine, ten or more LNA modifications. Without limitations, a LNA modification in the sense strand can be present at any position. In some embodiments, the sense strand comprises at least one, e.g., two, three, four, five, six, seven, eight, nine, ten or more LNA modifications and the antisense strand does not comprise a 2′-fluoro nucleotide at positions 3-9, counting from 5′-end.

The dsRNA molecule can comprise bridged nucleic acid (BNA). For example, the dsRNA molecule can comprise can comprise at least one, e.g., one, two, three, four, five, six, seven, eight, nine, ten, eleven, twelve, thirteen, fourteen, fifteen, sixteen, seventeen, eighteen, nineteen, twenty or more BNA modifications. Without limitations, the BNA nucleotides all can be present in one strand. In some embodiments, both the sense and the antisense strands comprise at least BNA modifications. The BNA modification can occur on any nucleotide of the sense strand or antisense strand. For instance, the BNA modification can occur on every nucleotide on the sense strand and/or antisense strand; each BNA modification can occur in an alternating pattern on the sense strand or antisense strand; or the sense strand and antisense strand both comprise BNA modifications in an alternating pattern. The alternating pattern of the BNA modifications on the sense strand can be the same or different from the antisense strand, and the alternating pattern of the BNA modifications on the sense strand can have a shift relative to the alternating pattern of the 2′-fluoro modifications on the antisense strand.

The antisense strand of the dsRNA molecule can comprise at least one, e.g., two, three, four, five, six, seven, eight, nine, ten or more BNA modifications. Without limitations, a BNA modification in the antisense strand can be present at any position.

The sense strand of the dsRNA molecule can comprise at least one, e.g., two, three, four, five, six, seven, eight, nine, ten or more BNA modifications. Without limitations, a BNA modification in the sense strand can be present at any position. In some embodiments, the sense strand comprises at least one, e.g., two, three, four, five, six, seven, eight, nine, ten or more BNA modifications and the antisense strand does not comprise a 2′-fluoro nucleotide at positions 3-9, counting from 5′-end.

The dsRNA molecule can comprise cyclohexene nucleic acid (CeNA). For example, the dsRNA molecule can comprise can comprise at least one, e.g., one, two, three, four, five, six, seven, eight, nine, ten, eleven, twelve, thirteen, fourteen, fifteen, sixteen, seventeen, eighteen, nineteen, twenty or more CeNA modifications. Without limitations, the CeNA nucleotides all can be present in one strand. In some embodiments, both the sense and the antisense strands comprise at least CeNA modifications. The CeNA modification can occur on any nucleotide of the sense strand or antisense strand. For instance, the CeNA modification can occur on every nucleotide on the sense strand and/or antisense strand; each CeNA modification can occur in an alternating pattern on the sense strand or antisense strand; or the sense strand and antisense strand both comprise CeNA modifications in an alternating pattern. The alternating pattern of the CeNA modifications on the sense strand can be the same or different from the antisense strand, and the alternating pattern of the CeNA modifications on the sense strand can have a shift relative to the alternating pattern of the 2′-fluoro modifications on the antisense strand.

The antisense strand of the dsRNA molecule can comprise at least one, e.g., two, three, four, five, six, seven, eight, nine, ten or more CeNA modifications. Without limitations, a CeNA modification in the antisense strand can be present at any position.

The sense strand of the dsRNA molecule can comprise at least one, e.g., two, three, four, five, six, seven, eight, nine, ten or more CeNA modifications. Without limitations, a CeNA modification in the sense strand can be present at any position. In some embodiments, the sense strand comprises at least one, e.g., two, three, four, five, six, seven, eight, nine, ten or more CeNA modifications and the antisense strand does not comprise a 2′-fluoro nucleotide at positions 3-9, counting from 5′-end.

In some embodiments, the dsRNA molecule comprises one or more overhang regions (i.e., single-stranded region) and/or capping groups of dsRNA molecule at the 3′-end, or 5′-end or both ends of a strand. Without limitations, the overhang can be 1-10 nucleotides in length, 1-6 nucleotides in length, 1-5 nucleotides in length, 1-4 nucleotides in length, 1-3 nucleotides in length, 2-6 nucleotides in length, 2-5 nucleotides in length 2-4 nucleotides in length, 2-3 nucleotides in length, or 1-2 nucleotides in length. The overhangs can be the result of one strand being longer than the other, or the result of two strands of the same length being staggered. The overhang can form a mismatch with the sequence being targeted or it can be complementary to the sequence being targeted or can be other sequence. The first and second strands can also be joined, e.g., by additional bases to form a hairpin, or by other non-base linkers. Without limitations the overhang can be present at the 3′-end of the sense strand, antisense strand or both strands.

In some embodiments, the dsRNA molecule comprises a single overhang. For example, the dsRNA molecule has a single overhang and the overhang is at least two, three, four, five, six, seven, eight, nine, or ten nucleotides in length. In some embodiments, the overhang is present at the 3′-end of the antisense strand. In some particular embodiments, the dsRNA comprises a two nucleotide overhang at the 3′-end of the antisense strand.

The dsRNA can also have a blunt end. For example, one end of the dsRNA is a blunt end and the other end has an overhang. Without limitations, the blunt end can be located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. In some embodiments, the dsRNA has a 2 nucleotide overhang on the 3′-end of the antisense strand and a blunt end at the 5′-end of the antisense strand.

In some other embodiments, the dsRNA molecule has two blunt ends, i.e., at both ends of the dsRNA.

The nucleotides in the overhang region of the dsRNA molecule can each independently be a modified or unmodified nucleotide including, but not limited to 2′-sugar modified, such as, 2′-Fluoro, 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, TT (or UU) can be an overhang sequence for either end on either strand. The 5′- or 3′-overhangs at the sense strand, antisense strand or both strands of the dsRNA molecule can be phosphorylated. In some embodiments, the overhang region contains two nucleotides having a phosphorothioate internucleotide linkage between the two nucleotides, where the two nucleotides in the overhang region can be the same or different.

The dsRNA molecule can comprise at least one, e.g., two, three, four, five, six, seven, eight, nine, ten or more phosphorothioate or methylphosphonate internucleotide linkage. The phosphorothioate or methylphosphonate internucleotide linkage modification can occur on any nucleotide of the sense strand or antisense strand or both in any position of the strand. For instance, the internucleotide linkage modification can occur on every nucleotide on the sense strand and/or antisense strand; each internucleotide linkage modification can occur in an alternating pattern on the sense strand or antisense strand; or the sense strand or antisense strand comprises both internucleotide linkage modifications in an alternating pattern. The alternating pattern of the internucleotide linkage modification on the sense strand can be the same or different from the antisense strand, and the alternating pattern of the internucleotide linkage modification on the sense strand can have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides can be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides can be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA molecule comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some exemplary dsRNA molecules, the sense strand can comprise 0, 1, 2, 3 or 4 phosphorothioate internucleotide linkages. For example, the sense strand comprises phosphorothioate internucleotide linkages between nucleotide positions 1 and 2, and between nucleotide positions 2 and 3.

In some exemplary dsRNA molecules, the antisense strand can comprise 1, 2, 3 or 4 phosphorothioate internucleotide linkages. For example, the sense strand comprises phosphorothioate internucleotide linkages between nucleotide positions 21 and 22, and between nucleotide positions 22 and 23. In an additional example, the antisense strand comprises phosphorothioate internucleotide linkages between nucleotide positions 1 and 2, between nucleotide positions 2 and 3, between nucleotide positions 21 and 22, and between nucleotide positions 22 and 23.

In some embodiments, the sense strand comprises phosphorothioate internucleotide linkages between nucleotide positions 1 and 2, and between nucleotide positions 2 and 3, and the antisense strand comprises phosphorothioate internucleotide linkages between nucleotide positions 21 and 22, and between nucleotide positions 22 and 23. For example, the sense strand comprises phosphorothioate internucleotide linkages between nucleotide positions 1 and 2, and between nucleotide positions 2 and 3, and the antisense strand comprises phosphorothioate internucleotide linkages between nucleotide positions 1 and 2, between nucleotide positions 2 and 3, between nucleotide positions 21 and 22, and between nucleotide positions 22 and 23.

### 5′-Modifications

In some embodiments, the dsRNA molecule can be 5′ phosphorylated or include a phosphoryl analog at the 5′ terminus. Exemplary 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HSXS)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)2(O)P-5′-CH2-), 5′-alkyletherphosphonates (R=alkylether=methoxymethyl (MeOCH2-), ethoxymethyl, etc., e.g. RP(OH)(O)—O-5′-). The modification can in placed in the antisense strand of a dsRNA molecule. For example, the antisense strand can comprise a 5′-vinylphosphonate nucleotide at 5′-end.

In some embodiments, the antisense comprises 5′-E-vinylphosphanate. In some embodiments, the antisense strand comprises 5′-E-vinylphosphanate and a nucleoside at position N−1 that reduces or inhibits activity of siRNA relative to a siRNA having the same antisense strand sequence but unmodified N−1 position and a nucleoside at position N−1 that reduces or inhibits activity of siRNA relative to a siRNA having the same antisense strand sequence but unmodified N−1 position

In some embodiments, the sense strand comprises a 5′-morpholino, a 5′-dimethylamino, a 5′-deoxy, an inverted abasic, or an inverted abasic locked nucleic acid modification at the 5′-end.

The linker between the Ig and the dsRNA molecule can be attached to the sense strand, antisense strand or both strands. Further, the linker can be conjugated at the 3′-end, 5′-end or both ends of a strand. For instance, the linker can be conjugated to the sense strand. In some embodiments, the linker is conjugated to the 3′-end of the sense strand. In some other embodiments, the linker is conjugated to the 3′-end of the sense strand.

Generally, the dsRNA has a melting temperature in the range from about 40° C. to about 80° C. For example, the dsRNA has a melting temperature with a lower end of the range from about 40° C., 45° C., 50° C., 55° C., 60° C. or 65° C., and upper end of the range from about 70° C., 75° C. or 80° C. In some embodiments, the dsRNA has a melting temperature in the range from about 55° C. to about 70° C. or in the range from about 60° C. to about 75° C. In some embodiments, the dsRNA has a melting temperature in the range from about 57° C. to about 67° C. In some particular embodiments, the dsRNA has a melting temperature in the range from about 60° C. to about 67° C. In some additional embodiments, the dsRNA has a melting temperature in the range from about 62° C. to about 66° C.

Without wishing to be bound by a theory, dsRNA molecules having a melting temperature of at least 60° C. are more effective in vivo and in vitro. Accordingly, in some embodiments, the dsRNA has a melting temperature of at least 60° C.

Without wishing to be bound by a theory, for the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 7 after in vivo administration. In some embodiments, at least 40/a, for example at least 45%, at least 50%, at least 55%, at least 60%, at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 15 after in vivo administration.

Without wishing to be bound by a theory, thermally destabilizing modifications in the seed region of the antisense strand (i.e., at positions 2-9 from the 5′-end of the antisense strand) can reduce or inhibit off-target gene silencing. Accordingly, in some embodiments, the antisense strand comprises at least one (e.g., one, two, three, four, five or more) thermally destabilizing modification of the duplex within the first 9 nucleotide positions of the 5′ region of the antisense strand. The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s).

In some embodiments, thermally destabilizing modification is located at position 2, 3, 4, 5, 6, 7, 8 or 9, or preferably at position 4, 5, 6, 7, or 8, from the 5′-end of the antisense strand. In some embodiments, the thermally destabilizing modification is located at position 2, 3, 4, 5 or 9 from the 5′-end of the antisense strand. In some other embodiments, the thermally destabilizing modification is located at position 6, 7 or 8 from the 5′-end of the antisense strand. In some particular embodiments, the thermally destabilizing modification is located at position 7 from the 5′-end of the antisense strand.

The thermally destabilizing modifications can include, but are not limited to, abasic modifications; mismatch with the opposing nucleotide in the opposing strand; and sugar modification such as 2′-deoxy modification or acyclic nucleotide, e.g., unlocked nucleic acids (UNA) or glycol nucleic acid (GNA).

Exemplary abasic modifications include, but are not limited to, the following:

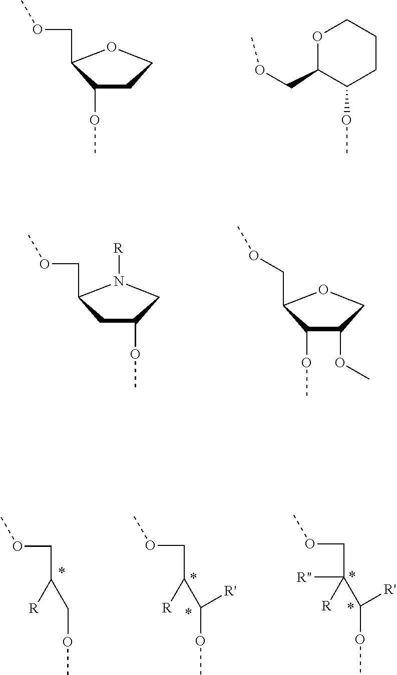

wherein R is H, Me, Et or OMe; R′ is H, Me, Et or OMe; R″ is H, Me, Et or OMe; and * represents either R, S or racemic.

Exemplary destabilizing sugar modifications include, but are not limited to the following:

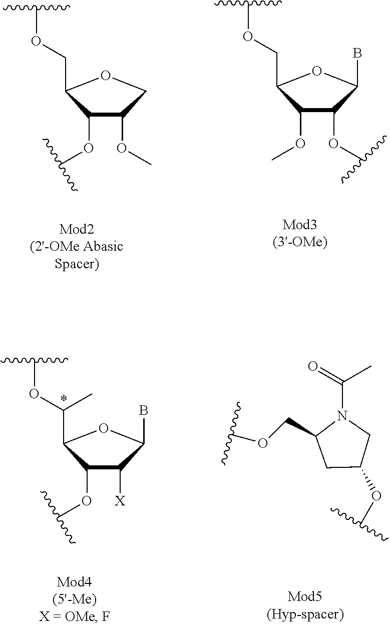

wherein B is a modified or unmodified nucleobase.

Additional sugar modifications include, but are not limited to the following:

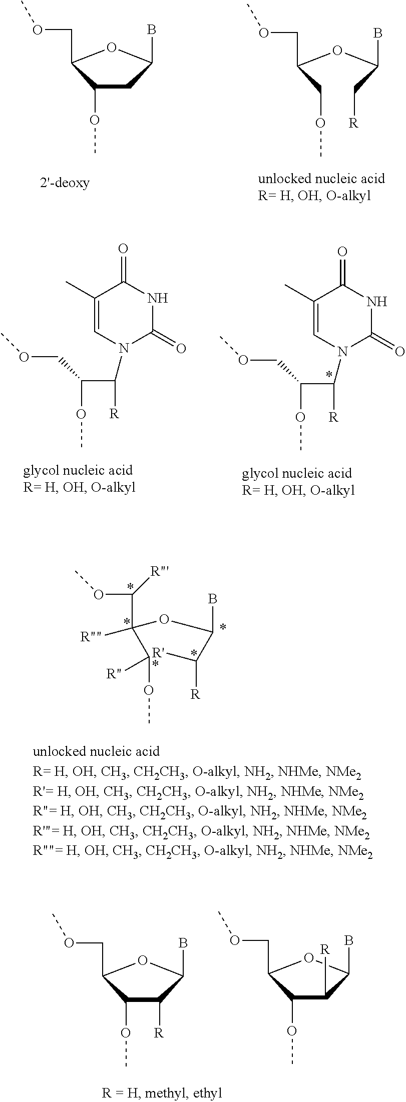

wherein B is a modified or unmodified nucleobase.

In some embodiments the thermally destabilizing modification is selected from the group consisting of:

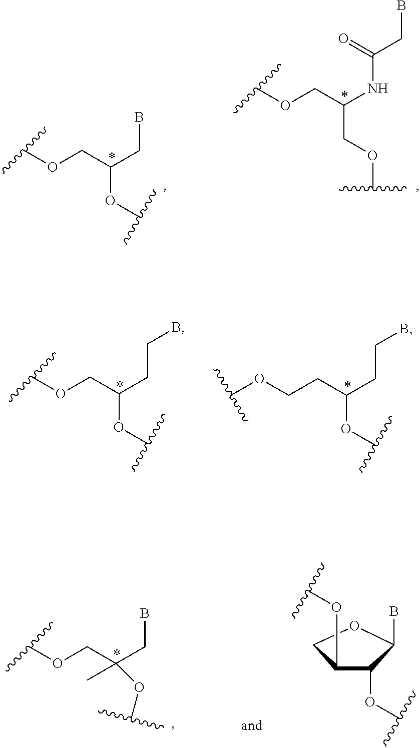

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′-04′, or C1′-04′) is absent and/or at least one of ribose carbons or oxygen (e.g., C1′, C2′, C3′, C4′ or 04′) are independently or in combination absent from the nucleotide. In some embodiments, acyclic nucleotide is

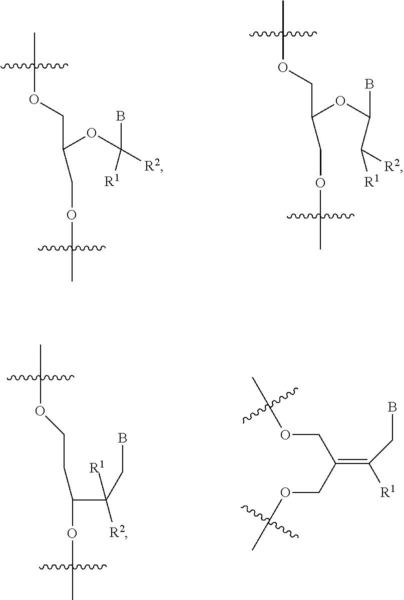

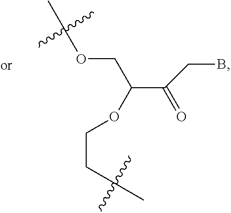

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

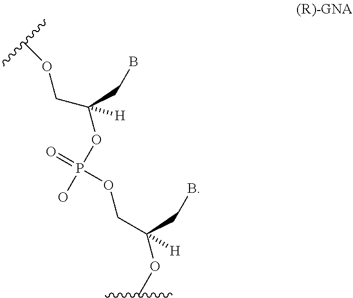

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule comprises at least one nucleobase in the mismatch pairing that is a 2′-deoxy nucleobase; e.g., the 2′-deoxy nucleobase is in the sense strand.

In some embodiments, the thermally destabilizing modification in the seed region of the antisense strand includes nucleotides with impaired W—C H-bonding to complementary base on the target mRNA. Exemplary, nucleotides with impaired W—C H-bonding to complementary base on the target mRNA include, but are not limited to, nucleotides comprising a nucleobase independently selected from the following:

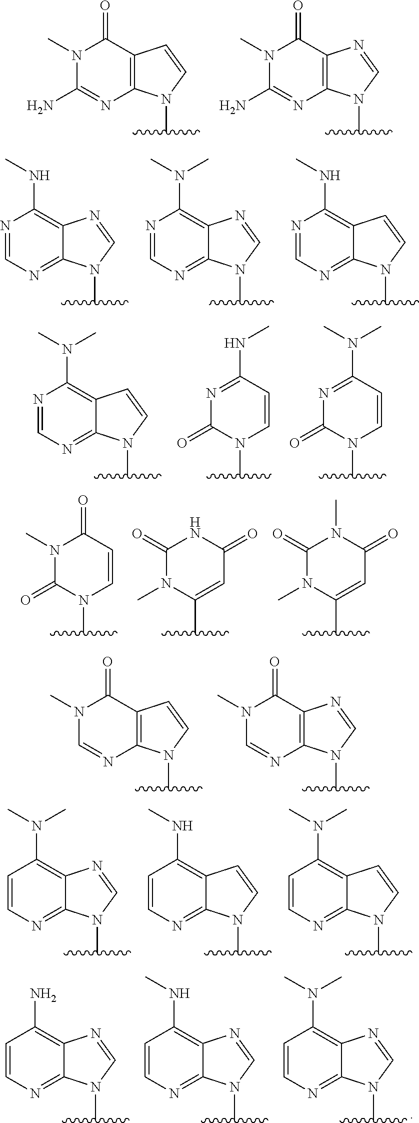

Additional examples of abasic nucleotide, acyclic nucleotide modifications (including UNA and GNA), and mismatch modifications have been described in detail in WO 2011/133876, which is herein incorporated by reference in its entirety.

The thermally destabilizing modifications can also include a universal nucleobase with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary such nucleobase modifications are:

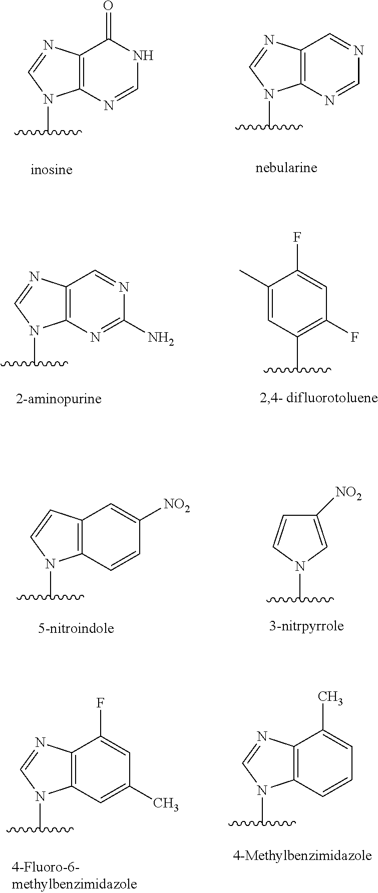

In some embodiments, the thermally destabilizing modification includes one or more □-nucleotide complementary to the base on the target mRNA, such as:

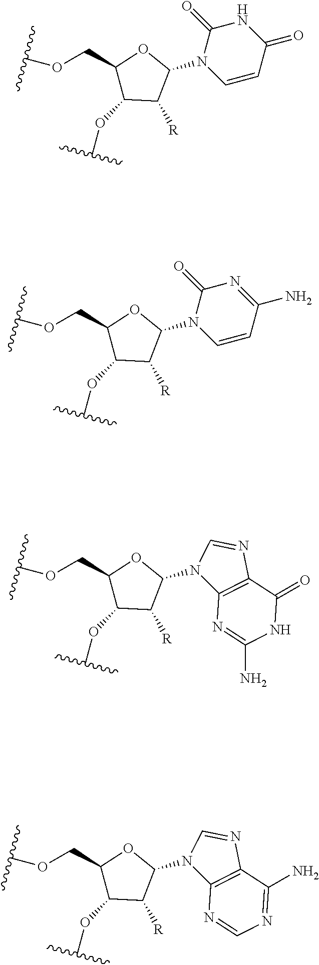

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages include, but are not limited to, the following:

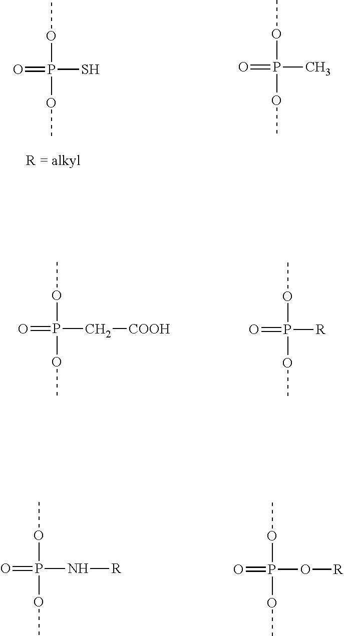

The alkyl for the R group can be a C1-C6alkyl. Specific alkyls for the R group include, but are not limited to methyl, ethyl, propyl, isopropyl, butyl, pentyl and hexyl.

In some embodiments, the destabilizing modification is selected from the following:

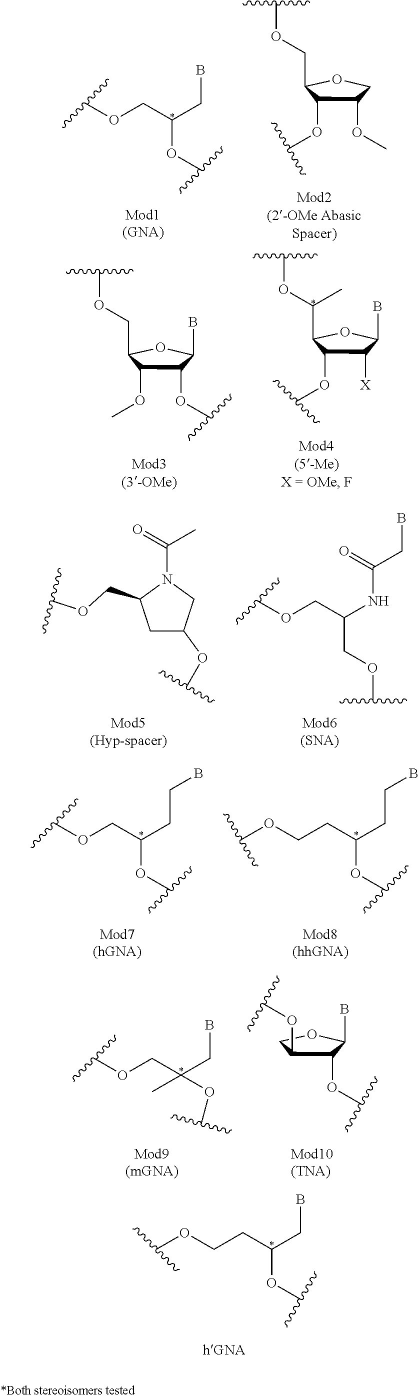

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions+1 and +2 from the position of the destabilizing modification.

In some embodiments, the sense strand does not comprise a thermally stabilizing modification in position opposite or complimentary to the thermally destabilizing modification of the duplex in the antisense strand.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotides at the 3′-end of the destabilizing modification, i.e., at positions+1 and +2 from the position of the destabilizing modification.

In some embodiments, the sense strand does not comprise a 2′-fluoro nucleotide in position opposite or complimentary to the thermally destabilizing modification of the duplex in the antisense strand.

In some embodiments, every nucleotide in the sense strand and/or the antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of monomers, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O-N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA molecule comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments, the dsRNA molecule comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule comprises at least one of the first 1, 2, 3, 4, or 5 base pairs within the duplex regions from the 5′-end of the antisense strand can be chosen independently from the group of A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

Without wishing to be bound by a theory, introducing 4′-modified and/or 5′-modified nucleotides to the 3′-end of a phosphodiester (PO), phosphorothioate (PS), and/or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of the dsRNA molecule. For instance, a 5′-alkylated nucleoside can be introduced at the 3′-end of a dinucleotide at any position of the dsRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, a 4′-modified nucleoside is introduced at the 3′-end of a dinucleotide at any position of the dsRNA. For instance, a 4′-alkylated nucleoside may be introduced at the 3′-end of a dinucleotide at any position of dsRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, a 5′-alkylated nucleoside is introduced at any position on the sense strand or antisense strand of the dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, a 4′-alkylated nucleoside is introduced at any position on the sense strand or antisense strand of the dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, a 4′-O-alkylated nucleoside is introduced at any position on the sense strand or antisense strand of the dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA molecule can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC. In some embodiments, the sense strand comprises a 2′-5′-linkage between positions N−1 and N−2, counting from 5′-end.

In some embodiments, the dsRNA molecule can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC. In some embodiments, the sense strand comprises a L sugar nucleotide at the 5′-end.

Exemplary dsRNA Embodiments

In some embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 6, 8, 9, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In still other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 6, 9, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 3′-end; and 2′-fluoro modifications at positions 7, 10, and 11 (counting from the 5′ end); and; and (ii) an antisense strand having a length of 23 nucleotides; 2′-fluoro modifications at positions 2, 6, 8, 9, 14, and 16 (counting from the 5′ end); phosphorothioate internucleotide linkages between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having: a length of 23 nucleotides; 2′-fluoro modifications at positions 2, 6, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In yet other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally a thermally destabilizing modification of the duplex at position 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having the linker attached to the 3′-end; and phosphorothioate internucleotide linkages between nucleotide positions 1 and 2, and between nucleotide positions 2 and 3 (counting from the 5′ end); and (ii) an antisense strand having: 2′-fluoro modifications at positions 2, 14, and 16 (counting from the 5′ end); and optionally, a thermally destabilizing modification of the duplex at position 6 or 7 (counting from the 5′ end).

In other embodiments, the dsRNA molecule comprises: (i) a sense strand having the linker attached to the 3′-end and phosphorothioate internucleotide linkages between nucleotide positions 1 and 2, and between nucleotide positions 2 and 3 (counting from the 5′ end); and (ii) an antisense strand having 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 3-end; 2′-fluoro modifications at positions 7, 10, and 11 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and at least one, e.g., one, two, three, four, five, six, seven, eight, nine, ten or more LNA modification; and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and a LNA modification at least at one, e.g., one, two or three of positions 1, 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); at least one, e.g., one, two, three, four, five, six, seven, eight, nine, ten or more LNA modifications; and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); and a LNA modification at least at one, e.g., one, two or three of positions 1, 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 3′-end; 2′-fluoro modifications at positions 7, 9, 10, and 11 (counting from the 5′ end); a LNA modification at least at one, e.g., one, two or three of positions 1, 2 and 3 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In still other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 6, 8, 9, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In still other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 6, 9, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 5′-end; and 2′-fluoro modifications at positions 7, 10, and 11 (counting from the 5′ end); and; and (ii) an antisense strand having a length of 23 nucleotides; 2′-fluoro modifications at positions 2, 6, 8, 9, 14, and 16 (counting from the 5′ end); phosphorothioate internucleotide linkages between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having: a length of 23 nucleotides; 2′-fluoro modifications at positions 2, 6, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 7 (counting from the 5′ end); and wherein the dsRNA molecule has a two nucleotide overhang at the 3′-end of the antisense strand, and a blunt end at the 5′-end of the antisense strand.

In yet other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally a thermally destabilizing modification of the duplex at position 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having the linker attached to the 5′-end; and phosphorothioate internucleotide linkages between nucleotide positions 1 and 2, and between nucleotide positions 2 and 3 (counting from the 5′ end); and (ii) an antisense strand having: 2′-fluoro modifications at positions 2, 14, and 16 (counting from the 5′ end); and optionally, a thermally destabilizing modification of the duplex at position 6 or 7 (counting from the 5′ end).

In other embodiments, the dsRNA molecule comprises: (i) a sense strand having the linker attached to the 5′-end and phosphorothioate internucleotide linkages between nucleotide positions 1 and 2, and between nucleotide positions 2 and 3 (counting from the 5′ end); and (ii) an antisense strand having 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 5′-end; 2′-fluoro modifications at positions 7, 10, and 11 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and at least one, e.g., one, two, three, four, five, six, seven, eight, nine, ten or more LNA modification; and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and a LNA modification at least at one, e.g., one, two or three of positions 1, 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); at least one, e.g., one, two, three, four, five, six, seven, eight, nine, ten or more LNA modifications; and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some other embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); and a LNA modification at least at one, e.g., one, two or three of positions 1, 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some embodiments, the dsRNA molecule comprises: (i) a sense strand having a length of 21 nucleotides; the linker attached to the 5′-end; 2′-fluoro modifications at positions 7, 9, 10, and 11 (counting from the 5′ end); a LNA modification at least at one, e.g., one, two or three of positions 1, 2 and 3 (counting from the 5′ end); and phosphorothioate internucleotide linkages between nucleotide positions 1 and 2, and between nucleotide positions 2 and 3 (counting from the 5′ end); and (ii) an antisense strand having a length of 23 nucleotides; 2′-fluoro modifications at positions 2, 14, and 16 (counting from the 5′ end); phosphorothioate internucleotide linkages between nucleotide positions 1 and 2, between nucleotide positions 2 and 3, between nucleotide positions 21 and 22, and between nucleotide positions 22 and 23 (counting from the 5′ end); and optionally, a thermally destabilizing modification of the duplex at position 5, 6 or 7 (counting from the 5′ end); and wherein the dsRNA molecule has a two nucleotide overhang at the 3′-end of the antisense strand, and a blunt end at the 5′-end of the antisense strand.

In some further embodiments of the exemplary dsRNA molecules described above, the sense strand comprises at least one, e.g., two, three, four, five, six, seven, eight, nine, ten, eleven, twelve, thirteen, fourteen, fifteen, sixteen or seventeen 2′-OMe modifications.

In some other further embodiments, of the exemplary dsRNA molecules described above, the antisense strand comprises at least one, e.g., two, three, four, five, six, seven, eight, nine, ten, eleven, twelve, thirteen, fourteen, fifteen, sixteen, seventeen, eighteen, nineteen or twenty 2′-OMe modifications.

In yet some other further embodiments, of the exemplary dsRNA molecules described above, the sense strand comprises at least one, e.g., two, three, four, five, six, seven, eight, nine, ten, eleven, twelve, thirteen, fourteen, fifteen, sixteen or seventeen 2′-OMe modifications, and the antisense strand comprises at least one, e.g., two, three, four, five, six, seven, eight, nine, ten, eleven, twelve, thirteen, fourteen, fifteen, sixteen, seventeen, eighteen, nineteen or twenty 2′-OMe modifications.

### Dual Variable Domain Immunoglobulins

Aspects of the invention include dual variable domain (DVD) immunoglobulin molecules (Ig). Generally, the dual variable domain immunoglobulin molecule comprises a first variable domain that binds to a target antigen, and a second variable domain that includes uniquely reactive residues that provide a site for covalent attachment of a linker molecule. Further, the DVD immunoglobulin molecule includes two identical light chains, as well as two identical heavy chains. Each light chain and each heavy chain includes an N-terminus and a C-terminus, Assembly of two light chains and two heavy chains results in the formation of a DVD immunoglobulin molecule, with various inter-chain and intra-chain disulfide bonds stabilizing the interactions of the light and heavy chains.

Each light chain includes a first and a second variable domain, designated as V_{L}1 and V_{L}2, as well as a constant domain, designated as CL. In some embodiments, a light chain comprises a kappa light chain. In some other embodiments, a light chain comprises a lambda light chain.

In some embodiments, the second variable domain comprises a single, uniquely reactive lysine residue that provides a site for covalent attachment of a linker molecule.

In some other embodiments, the second variable domain comprises a single, uniquely reactive arginine residue that provides a site for covalent attachment of a linker molecule.

In some embodiments, each heavy chain independently includes a first and a second variable domain, designated as V_{H}1 and V_{H}2, as well as a constant domain designated as C_{H}1, followed by heavy chain Fe region domains. In some embodiments, Fc region domains on a heavy chain can include Fc region domains that are specific to a particular immunoglobulin type or subtype, including but not limited to Fe regions from an IgG (such as an IgG1, IgG2, IgG3 or IgG4), IgA (such as an IgA1 or IgA2), IgM, IgE or IgD antibody. For example, an immunoglobulin belongs to the IgG class, and the heavy chain comprises a γ heavy chain. In some embodiments, an immunoglobulin belongs to the IgG1 class, and the heavy chain comprises a γ1 heavy chain. In some other embodiments, an immunoglobulin belongs to the IgG2 class, and the heavy chain comprises a γ2 heavy chain. In still some embodiments, an immunoglobulin belongs to the IgG3 class, and the heavy chain comprises a γ3 heavy chain. In some embodiments, an immunoglobulin belongs to the IgG4 class, and the heavy chain comprises a γ4 heavy chain.

In some embodiments, an immunoglobulin belongs to the IgA class. For example, an immunoglobulin belongs to the IgA class and a heavy chain comprises an a heavy chain. In some embodiments, an immunoglobulin belongs to the IgA1 class, and a heavy chain comprises an al heavy chain. In some embodiments, an immunoglobulin belongs to the IgA2 class, and a heavy chain comprises an a2 heavy chain.

In some embodiments, an immunoglobulin belongs to the IgD class and a heavy chain comprises a δ heavy chain. In some embodiments, an immunoglobulin belongs to the IgE class, and a heavy chain comprises an a heavy chain. In some embodiments, an immunoglobulin belongs to the IgM class, and a heavy chain comprises a heavy chain.

In some embodiments, an immunoglobulin molecule can comprise a native polypeptide sequence that occurs in nature.

The organization of the variable and constant domains along the light chain generally proceeds from the N-terminus to the C-terminus as V_{L}1-V_{L}2-C_{L}. However, in some embodiments, the organization of the variable domains on the light chain can be reversed, such that the organization from N- to C-terminus is V_{L}2-V_{L}1-C_{L} This same organization applies to binding fragments of the subject DVD immunoglobulins, wherein the organization from N- to C-terminus can be V_{L}1-V_{L}2 or V_{L}2-V_{L}1. In some embodiments, the light chain does not comprise a second variable domain. For example, the organization of the variable and constant domains along a light chain can be organized such that the sequence of the domains along the light chain proceeds from N- to C-terminus as V_{L}1-C_{L}1 or V_{L}-C_{L}.

Similarly, the organization of the variable and constant domains along the heavy chain generally proceeds from the N-terminus to the C-terminus, such as V_{H}1-V_{H}2-C_{H}1, V_{H}1-C_{H}1-V_{H}2-C_{H}1, V_{H}1-V_{H}2-C_{H}1-FC or V_{H}1-C_{H}1-V_{H}2-C_{H}1-FC, but can be modified to mirror the organization of the domains on a light chain so that the appropriate domains on a light chain are paired with the appropriate domains on a heavy chain when the immunoglobulin molecule, or binding fragment thereof, is assembled.

In some embodiments, the organization of the variable and constant domains along a light and heavy chain can be organized such that the sequence of the domains along a light chain proceeds from N- to C-terminus as V_{L}1-V_{L}2-C_{H}1, and the organization of domains along a heavy chain proceeds from N- to C-terminus as V_{H}1-V_{H}2-C_{L}-FC. This particular organization is referred to as a CrossMAb organization, and is described in detail in Klein et al., mAbs 4, 653-663 (2012), the disclosure of which is incorporated by reference herein in its entirety. In some embodiments, a CrossMAb organization can be used to generate bispecific DVD immunoglobulins, which are described further below.

In some embodiments, the organization of the variable and constant domains along a light chain can be organized such that the sequence of the domains along the light chain proceeds from N- to C-terminus as V_{L}1-C_{H}1 and the organization of the variable and constant domains along a heavy chain can be organized such that the sequence of the domains along a heavy chain proceeds from N- to C-terminus as V_{H}1-V_{H}2-C_{H}1, V_{H}1-C_{H}1-V_{H}2-C_{H}1, V_{H}1-V_{H}2-C_{H}1-FC or V_{H}1-C_{H}1-V_{H}2-C_{H}1-FC.

In some embodiments, the organization of the variable and constant domains along a light chain can be organized such that the sequence of the domains along the light chain proceeds from N- to C-terminus as V_{H}1-C_{H}1 and the organization of the variable and constant domains along a heavy chain can be organized such that the sequence of the domains along a heavy chain proceeds from N- to C-terminus as V_{H}1-C_{H}1-V_{H}2-C_{H}1 or V_{H}1-C_{H}1-V_{H}2-C_{H}1-FC.

It is noted that different domains, e.g., two variable domains, a variable domain and a constant domain, a variable domain and an Fe domain, and/or a constant domain and an Fe domain can be linked together via a linker. The linker can be a chemical linker, a single peptide bond (e.g., linked directly to each other) or a peptide linker containing one or more amino acid residues (e.g. with an intervening amino acid or amino acid sequence between the domain. The term “peptide linker” as used herein denotes a peptide with amino acid sequences, which is in some embodiments of synthetic origin. It is noted that peptide linkers may affect folding of a given fusion protein, and may also react/bind with other proteins, and these properties can be screened for by known techniques. A peptide linker can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. Conversely, a peptide linker can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids.

In some embodiments of the various aspects described herein, the peptide linker comprises from about 5 amino acids to about 40 amino acids. For example, the peptide linker can comprise from about 5 amino acids to about 35 amino acids, from about 5 amino acids to 30 amino acids, or from about 5 amino acids to about 25 amino acids. In some embodiments of the various aspects described herein, the peptide linker comprises 1, 2, 3, 4, 5, 6, 7, 8, 9, 10, 11, 12, 13, 14, 15, 16, 17, 18, 19, 20, 21, 22, 23, 24 or 25 amino acids.

Exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyₓSer)ₙ, where x is 2, 3, 4 or 5, and n is 1, 2, 3, 4, 5, 6, 7, 8, 9 or 10. In some embodiments of the various aspects described herein, x is 3 and n is 1, 2, 3 or 4. In some embodiments of the various aspects described herein, x is 3 and n is 3 or 4. In some embodiments of the various aspects described herein, x is 4 and n is 1, 2, 3 or 4. In some embodiments of the various aspects described herein, x is 4 and n is 1, 2 or 3.

Additional exemplary peptide linker sequences that can be used are provided in U.S. Pat. No. 7,612,181, and PCT Publication No. WO2017/049139, the contents of both of which are incorporated herein by reference in their entireties.

In some embodiments, the linker comprises an amino acid sequence selected from the group consisting of ASTKGP (SEQ ID NO: 1), TVAAPSVFIFPP (SEQ ID NO: 2), G₄S (SEQ ID NO: 3), (G₄S)₂ (SEQ ID NO: 4), (G₄S)₃ (SEQ ID NO: 5), EPKSCDG₄S (SEQ ID NO: 6), EPKSCD(G₄S)₂ (SEQ ID NO: 7), EPKSCD(G₄S)₃ (SEQ ID NO: 8), and any combinations thereof.

In some embodiments, the first and second variable domains are linked along their light chain or heavy chain by a peptide linker sequence. A peptide linker sequence can be a single amino acid or a polypeptide sequence. In some embodiments, the first and second variable domain are linked by a peptide linker, where the peptide linker sequence is ASTKGP (SEQ ID NO: 1), TVAAPSVFIFPP (SEQ ID NO: 2), G₄S (SEQ ID NO: 3), (G₄S)₂ (SEQ ID NO: 4), (G₄S)₃ (SEQ ID NO: 5), EPKSCDG₄S (SEQ ID NO: 6), EPKSCD(G₄S)₂ (SEQ ID NO: 7), EPKSCD(G₄S)₃ (SEQ ID NO: 8), or any combinations thereof

Additional peptide linker sequences that can be used to link two domains, e.g., a first and second variable domain of the subject DVD immunoglobulins are provided in U.S. Pat. No. 7,612,181, and PCT Publication No. WO2017/049139, the contents of both of which are incorporated herein by reference in their entireties.

In some embodiments, the DVD immunoglobulin molecules comprise a first variable domain with antigen binding functionality. V_{L}1 and V_{H}1 sequences of the subject DVD immunoglobulin molecules are selected to specifically bind to a target, such as, a cell-surface marker or antigen. One of skill in the art will realize that antigens are known for virtually any type of cell. Thus, V_{L}1 and V_{H}1 sequences of the subject DVD immunoglobulin molecules are selected to specifically bind to virtually any known antigen on virtually any type of cell.

In some embodiments, the V_{L}1 and V_{H}1 sequences of the subject DVD immunoglobulin molecules are selected to specifically bind to an antigen on a tumor cell. Immunoglobulins can exert antitumor effects by inducing apoptosis, redirected cytotoxicity, interfering with ligand-receptor interactions, or preventing the expression of proteins that are critical to a neoplastic phenotype. In addition, immunoglobulins can target components of the tumor microenvironment, perturbing vital structures such as the formation of tumor-associated vasculature. Immunoglobulins can also target receptors whose ligands are growth factors, such as the epidermal growth factor receptor, thus inhibiting binding of natural ligands that stimulate cell to targeted tumor cells. Alternatively, immunoglobulins can induce ADCC, ADCP or CDC.

Exemplary, tumor-associated binding targets that can be targeted by the first variable domain of the DVD immunoglobulin molecule include, but are not limited to, CD138, BCMA, SLAMF7, HER2 (ERBB2), FOLR1, FOLR2, CD19, CD79A, CD79B, ROR1, ROR2, FCRM, CS1, GPA33, PSMA, Siglec-1, Siglec-4, Siglec-5, Siglec-6, Siglec-7, Siglec-8, Siglec-9. Siglec-10, MSLN, CD52, CD20, CD3, CD4, CD8, CD20. CD21, CD22, CD23, CD30, CD33, CD38, CD44, CD56, CD70, BMP6, IL12A, IL1A, IL1B, IL2, IL24, INHA, TNF, TNFSFIO, BMP6, EGF, FGF1, FGF10, FGF11, FGF12, FGF13, FGF14, FGF16, FGF17, FGF18, FGF19, FGF2, FGF20, FGF2l, FGF22, FGF23, FGF3, FGF4, FGF5, FGF6, FGF7, FGF8, FGF9, GRP, IGF1, IGF2, IL12A, ILIA, IL1B, IL2, INHA, TGFA, TGFB1, TGFB2, TGFB3, VEGF, CDK2, EGF, FGF10, FGF18, FGF2, FGF4, FGF7, IGF1, IGF1R, IL2, VEGF, BCL2, CD164, CDKN1A, CDKN1B, CDKN1C, CDKN2A, CDKN2B, CDKN2C, CDKN3, GNRH1, TGFBP6, ILIA, IL1B, ODZ1, PAWR, PLG, TGFB1 II, AR, BRCA1, CDK3, CDK4, CDK5, CDK6, CDK7, CDK9, E2F1, EGFR (ERBB1), HER3 (ERBB3), HER4 (ERBB4), ENO1, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, R1D1, R1H3, NR1I3, R2F6, R4A3, ESR1, ESR2, R0B1, R0B2, R1D2, R1H2, R1H4, R1I2, R2C1, R2C2, R2E1, R2E3, R2F1, R2F2, R3C1, R3C2, R4A1, R4A2, R5A1, R5A2, R6A1, PGR, RARB, FGF1, FGF2, FGF6, KLK3, KRT1, APOC1, BRCA1, CLHGA, CHGB, CLU, COL1A1, COL6A1, EGF, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, G RH1, IGF1, IGF2, IGFBP3, IGFBP6, IL12A, ILIA, IL1B, IL2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TTMP3, CD44, CDH11, CDH10, CDH19, CDH20, CDH7, CDH9, CDH1, CDH10, CDH13, CDH1S, CDH19, CDH20, CDH7, CDH8, CDH9, ROB02, CD44, ILK, ITGA1, APC, CD164, COL6A1, MTSS1, PAP, TGFBIII, AG 2, AIG1, AKAP1, AKAP2, CANT1, CAV1, CDH112, CLDN3, CLN3, CYB5, CYC1, DAB21P, DES, DNCL1, ELAC2, EN02, EN03, FASN, FLJ12584, FLJ25530, GAGEB1, GAGEC1, GGT1, GSTP1, HIP1, HUMCYT2A, IL29, K6H1F, KAI1, KRT2A, MIB 1, PARTI, PATE, PC A3, PIAS2, PK3CG, PPID, PR1, PSCA, SLC2A2, SLC33A1, SLC43A1, STEMP, STEAP2, TPM1, TPM2, TRPC6, ANGPT1, ANGPT2, ANPEP, ECGF1, EREG, FGF1, FGF2, FIGF, FLT1, JAG1, KDR, LAMA5, NRP1, NRP2, PGF, PLXDC1, STAB1, VEGF, VEGFC, ANGPTL3, BAI1, COL4A3, IL8, LAMA5, NRP1, NRP2, STAB1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kipl), CDKN2A (p16JNK4a), COL6A1, CTNNB1 (b-catenin), CTSB (cathepsin B), ESR1, ESR2, F3 (TF), FOSL1 (FRA-1), GAT A3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MK167 (Ki-67), NGFB (NGF), NGFR, NME1 (NM23A), PGR, PLAU (uPA), PTEN, SERPINB5 (maspin), SERPF E1 (PAI-1), TGFA, THBS1 (thrombospondin-1), TIE (Tie-1), TNTRSF6 (Fas), TNTSF6 (FasL), TOP2A (topoisomerase Iia), TP53, AZGP1 (zinc-a-glycoprotein), BPAG1 (plectin), CDKN1A (p21Wapl/Cipl), CLDN7 (claudin-7), CLU (clusterin), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-II), MUC (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRRIB (Sprl), THBS1, THBS2, THBS4, and TNFAIP2 (B94).

The amino acid sequences of a first variable domain region, which provides antigen binding functionality, can include chimeric, humanized, or human amino acid sequences. Any suitable combination of such sequences can be incorporated into a first variable domain of the DVD immunoglobulin molecule.

Antigen-binding variable region sequences can be selected from various monoclonal antibodies capable of binding specific targets and well known in the art. These include, but are not limited to anti-TNT antibody (U.S. Pat. No. 6,258,562), anti-IL-12 and or anti-IL-12p40 antibody (U.S. Pat. No. 6,914,128); anti-IL-18 antibody (US 2005/0147610 A1), anti-C5, anti-CBL, anti-CD147, anti-gp120, anti-VLA4, anti-CD1 1a, anti-CD18, anti-VEGF, anti-CD40L, anti-Id, anti-ICAM-1, anti-CXCL13, anti-CD2, anti-EGFR, anti-TGF-beta 2, anti-E-selectin, anti-Fact VII, anti-Her2/neu, anti-F gp, anti-CD1 1/18, anti-CD14, anti-ICAM-3, anti-CD80, anti-CD4, anti-CD3, anti-CD23, anti-beta2-integrin, anti-alpha4beta7, anti-CD52, anti-HILA DR, anti-CD22, anti-CD20, anti-MIF, anti-CD64 (FcR), anti-TCR alpha beta, anti-CD2, anti-Hep B, anti-CA 125, anti-EpCAM, anti-gp120, anti-CMV, anti-gpllbllla, anti-IgE, anti-CD25, anti-CD33, anti-HLA, anti-VNRintegrin, anti-IL-1 alpha, anti-IL-1beta, anti-IL-1 receptor, anti-IL-2 receptor, anti-IL-4, anti-IL4 receptor, anti-IL5, anti-IL-5 receptor, anti-IL-6, anti-IL-8, anti-IL-9, anti-L-13, anti-IL-13 receptor, anti-IL-17, and anti-IL-23 (see Presta L G. 2005 Selection, design, and engineering of therapeutic antibodies J Allergy Chin Immunol. 116:731-6 and Clark, M., “Antibodies for Therapeutic Applications,” Department of Pathology, Cambridge University, UK, 15 Oct. 2000, published online at M. Clark's home page at the website for the Department of Pathology, Cambridge University).

Antigen-binding variable region sequences can also be selected from various therapeutic antibodies approved for use, in clinical trials, or in development for clinical use. Such therapeutic antibodies include, but are not limited to, RITUXAN®, IDEC/Genentech/Roche) (see for example U.S. Pat. No. 5,736,137), a chimeric anti-CD20 antibody approved to treat Non-Hodgkin's lymphoma; HUMAX-CD20®, an anti-CD20 currently being developed by Genmab, an anti-CD20 antibody described in U.S. Pat. No. 5,500,362, AME-133 (Applied Molecular Evolution), hA20 (Immunomedics, Inc.), 1HumaLYM (Intracel), and P1R070769 (PCT/US2003/040426, entitled “Immunoglobulin Variants and Uses Thereof), trastuzumab (HERCEPTF®, Genentech) (see for example U.S. Pat. No. 5,677,171), a humanized anti-Her2/neu antibody approved to treat breast cancer; pertuzumab (rhuMab-2C4, OMNITARG®), currently being developed by Genentech; an anti-Her2 antibody described in U.S. Pat. No. 4,753,894; cetuxirnab (ERBITUX®, Imelone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HUMAX-EGFR™ (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3): 129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1):71-81); mAb-806 (Ludwig Institute for Cancer Research, Memorial Sloan-Kettering) (Jungbluth et al. 2003, Proc Natl Acad Sci USA. 100(2):639-44); KSB-102 (KS Biomedix); MR-1 (IVAX, National Cancer Institute) (PCT WO 0162931A2); and SCIOO (Scancell) (PCT WO 01/88138); alemtuzumab (CAMPATH®, Millennium), a humanized monoclonal antibody currently approved for treatment of B-cell chronic lymphocytic leukemia; muroromab-CD3 (Orthoclone OKT3®), an anti-CD3 antibody developed by Ortho Biotech/Johnson & Johnson, ibritumomab tiuxetan (ZEVALIN®), an anti-CD20 antibody developed by IDEC/Schering AG, gemtuzumab ozogamicin (MYLOTARG®), an anti-CD33 (p67 protein) antibody developed by Celltech/Wyeth, alefacept (AMEVIVE®), an anti-LFA-3 Fc fusion developed by Biogen), abeiximab (REOPRO®), developed by Centocor/Lilly, basilixinab (SFMULECT®), developed by Novartis, palivizumab (SYNAGIS®), developed by Medimmune, intliximab (REMICADE®), an anti-TNF alpha antibody developed by Centocor, adalimunab (HUMIRA®), an anti-TNTalpha antibody developed by Abbott, HUMICADE®, an anti-TNF alpha antibody developed by Celltech, etanercept (ENBREL®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtunomab (R1549, 90Y-muHMFG1), an anti-MUCl in development by Antisoma, Therex (R1550), an anti-MUCl antibody being developed by Antisona, AngioMab (AS 1405), being developed by Antisoma, HuBC-1, being developed by Antisoma, Thioplatin (AS1407) being developed by Antisoma, ANTEGREN® (natalizumab), an anti-alpha-4-beta-1 (VLA4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-P2 antibody being developed by Cambridge Antibody Technology, J695, an anti-IL-12 antibody being developed by Cambridge Antibody Technology and Abbott, CAT-192, an anti-TGFpi antibody being developed by Cambridge Antibody Technology and Genzyme, CAT-213, an anti-Eotaxin1 antibody being developed by Cambridge Antibody Technology, LYMPHOSTAT-B® an anti-Blys antibody being developed by Cambridge Antibody Technology and Human Genome Sciences Inc., TRAIL-RlmAb, an anti-TRAIL-R1 antibody being developed by Cambridge Antibody Technology and Human Genome Sciences, Inc., AVASTIN® bevacizurmab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, XOLAIR® (Omalizumab), an anti-IgE antibody being developed by Genentech, RAPTIVA® (Efalizumab), an anti-CD 1 1a antibody being developed by Genentech and Xoma, MLN-02 Antibody (formerly LDP-02), being developed by Genentech and Millennium Pharmaceuticals, HUMAX CD4®, an anti-CD4 antibody being developed by Genmab, HUMDTAX™-IL15, an anti-IL15 antibody being developed by Genmab and Amgen, HUMAX™-Inflam, being developed by Genmab and Medarex, HUMAX™-Cancer, an anti-Heparanase I antibody being developed by Genmab and Medarex and Oxford GlycoSciences, HUMAX™-Lymphoma, being developed by Genmab and Amgen, HUMAX™-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, FMC-1C11, an anti-KDR antibody being developed by Imclone, DC 101, an anti-flk-1 antibody being developed by Imclone, anti-VE cadherin antibodies being developed by Imclone, CEA-CIDE® (labetuzumab), an anti-carcinoembryonic antigen (CEA) antibody being developed by Immunomedics, LYMPHOCIDE® (Epratuzumab), an anti-CD22 antibody being developed by Immunomedics, AFP-Cide, being developed by Immunomedics. MyelomaCide, being developed by Immunomedics, LkoCide, being developed by Immunomedics, ProstaCide, being developed by Immunomedics, MDX-010, an ani-CTLA4 antibody being developed by Medarex, MDX-060, an anti-CD30 antibody being developed by Medarex, MDX-070 being developed by Medarex, MDX-018 being developed by Medarex, OSIDEM® (IDM-1), and anti-Her2 antibody being developed by Medarex and Immuno-Designed Molecules, HUMAX®-CD4, an anti-CD4 antibody being developed by Medarex and Genmab, HuMax-IL15, an anti-IL115 antibody being developed by Medarex and Genmab, CNTO 148, an anti-TNFa antibody being developed by Medarex and Centocor/J&J, CNTO 1275, an anti-cytokine antibody being developed by Centocor/J&J, MORIN and MORT 02, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, NUVION® (visilizumab), an anti-CD3 antibody being developed by Protein Design Labs, HUZAF®, an anti-gamma interferon antibody being developed by Protein Design Labs, Anti-a 5β1 Integrin, being developed by Protein Design Labs, anti-IL-12, being developed by Protein Design Labs, JNG-1, an anti-Ep-CAM antibody being developed by Xoma, XOLAIR® (Omalizumab) a humanized anti-IgE antibody developed by Genentech an Novartis, and MLN01 an anti-PBeta2 integrin antibody being developed by Xoma. Contents of all of the above-cited references in this paragraph are expressly incorporated herein by reference in their entireties.

In some embodiments, the DVD immunoglobulin molecule comprises a second variable domain from a 38C2 antibody, which includes a reactive lysine residue. A 38C2 antibody is described, for example, in U.S. Pat. No. 8,252,902, the disclosure of which is herein incorporated by reference in its entirety. Briefly, a heavy chain variable region of the 38C2 antibody includes a single, uniquely reactive lysine residue that can react with a linker, thereby providing an attachment point for conjugation with a drug moiety. As such, immunoglobulin molecules that include a variable domain of the 38C2 antibody contain two such attachment points (one on each heavy chain) that can be used for conjugation with a drug moiety. Once a reactive lysine residue has been conjugated to a linker, the binding functionality of the 38C2 variable domain is lost, meaning that the variable domain no longer binds to a target. As such, while not being limited by any particular theory, a variable domain of 38C2 antibody that is used in the subject DVD immunoglobulin molecules provides an attachment point for conjugation, but does not provide antigen binding functionality. In some embodiments, the DVD immunoglobulin molecule comprises a second variable domain from a 38C2 antibody as described in WO2017/049139, content of which is incorporated herein by reference in its entirety.

An exemplary amino acid sequence of light chain variable (V_{L}) domain is as follows:

(SEQ ID NO: 9)
ELQMTQSPSSLSASVGDRVTITCRSSQSLLHTYGSPYLNWYLQKPGQSPK
LLIYKVSNRFSGVPSRFSGSGSGTDFTLTISSLQPEDFAVYFCSQGTHLP
YTFGGGTKVEIK

An exemplary amino acid sequence of heavy chain variable (V_{H}) domain is as follows:

(SEQ ID NO: 10)
EVQLVESGGGLVQPGGSLRLSCAASGFTFSNYWMSWVRQSPEKGLEWVSE
IRLRSDNYATHYAESVKGRFTISRDNSKNTLYLQMNSLRAEDTGIYYCKT
YFYSFSYWGQGTLVTVSS.

An exemplary amino acid sequence of light chain variable (V_{L}) domain of an anti-BCMA Fab (VOO1 Fab) is as follows:

(SEQ ID NO: 11)
DVVMTQTPSSVPAAVGGTVTINCQASQSIDSNLAWFQQKPGQPPNLLIYD
ASTLASGVPSRFKGSAGKQFTLTISGVQREDAATYYCLGSYSRTEKAFGA
GTKVEIK

An exemplary amino acid sequence of heavy chain variable (V_{H}) domain of VOO1 Fab is as follows:

(SEQ ID NO: 12)
QEQLEESGGRLVTPGTPLTLTCTVSGFSLSNYHMSWVRQAPGKGLEWIGF
ITSGGSTYYASWAKGRFTISRTSTTVDLKITSPTTEDTATYFCARWNGYG
GNMWGPGTLVTVSS

An exemplary amino acid sequence of light chain variable (V_{L}) domain of humanized VOO1 Fab is as follows:

(SEQ ID NO: 13)
DIQMTQSPSSLSASVGDRVTITCQASQSIDSNLAWYQQKPGKVPKLLIYD
ASTLASGVPSRFSGSGSGTDFTLTISSLQPEDVATYYCLGSYSRTEKAFG
GGTKVEIK

An exemplary amino acid sequence of heavy chain variable (V_{H}) domain of humanized VOO1 Fab is as follows:

(SEQ ID NO: 14)
EVQLVESGGGLVQPGGSLRLSCAASGFTLSNYHMSWVRQAPGKGLEWVSF
ITSGGSTYYASWAKGRFTISRDNSKNTLYLQMNSLRAEDTAVYYCARWNG
YGGNMWGQGTLVTVS

An exemplary amino acid sequence of light chain variable (V_{L}, shown in bold) and constant (C_{L}) domains of a humanized 38C2 antibody is as follows:

(SEQ ID NO: 15)
ELQMTQSPSSLSASVGDRVTITCRSSQSLLHTYGSPYLNWYLQKPGQSPK
LLIYKVSNRFSGVPSRFSGSGSGTDFTLTISSLQPEDFAVYFCSQGTHLP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

An exemplary amino acid sequence of heavy chain variable (V_{H}, shown in bold) and constant (C_{H}1, hinge, C_{H}2, and C_{H}3) domains of a humanized 38C2 antibody is as follows:

(SEQ ID NO: 16)
EVQLVESGGGLVQPGGSLRLSCAASGFTFSNYWMSWVRQSPEKGLEWVSE
IRLRSDNYATHYAESVKGRFTISRDNSKNTLYLQMNSLRAEDTGIYYCKT
YFYSFSYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGA.

In some embodiments, the DVD immunoglobulin molecule includes a light chain variable domain sequence of a humanized 38C2 antibody (SEQ ID NO: 9 and SEQ ID NO: 15) as a V_{L}² domain sequence. In some embodiments, the DVD immunoglobulin molecule includes a VL2 domain sequence that is substantially similar to SEQ ID NO: 9 or SEQ ID NO: 15, for example, has at least about 80% amino acid sequence identity, alternatively has at least about 81%, 82%, 83%, 84%, 85%, 86%, 87%, 88%, 89%, 90%, 91%, 92%, 93%, 94%, 95%, 96%, 97%, 98%, or 99% amino acid sequence identity to SEQ ID NO: 9 or SEQ ID NO: 15.

In some embodiments, the DVD immunoglobulin molecule includes a heavy chain variable domain sequence of a humanized 38C2 antibody (SEQ ID NO: 10 and SEQ ID NO: 16) as a V_{H}2 domain sequence. In some embodiments, the DVD immunoglobulin molecule includes a V_{H}² domain sequence that is substantially similar to SEQ ID NO: 10 or SEQ ID NO: 16, for example, has at least about 80%) amino acid sequence identity, alternatively has at least about 81%, 82%, 83%, 849, 85%, 86%87%, 88%, 89%, 90%, 91%, 92%, 93%, 94%, 95%, 96%, 97%, 98%, or 99% amino acid sequence identity to SEQ ID NO: 10 or SEQ ID NO: 16, and includes a reactive lysine residue.

The DVD immunoglobulin molecule can encompass chimeric, humanized and human immunoglobulin sequences, and in some embodiments, can contain any mixture thereof. For example, in some embodiments, a DVD immunoglobulin molecule can include a chimeric first variable domain, and can include a human second variable domain. In some embodiments, a DVD immunoglobulin molecule can include a humanized first variable domain, and can contain a human second variable domain. Any suitable combination of chimeric, humanized and human immunoglobulin sequences can be utilized in the subject DVD immunoglobulin molecules.

In some embodiments, a DVD immunoglobulin described herein can be modified with respect to effector function of the immunoglobulin. This can be achieved by introducing one or more amino acid substitutions in an Fc region of an immunoglobulin. Alternatively, or additionally, cysteine residue(s) can be introduced in the Fc region, thereby allowing inter-chain disulfide bond formation in this region. An immunoglobulin thus generated can have improved internalization capability and/or increased effector function. See Caron et al., J. Exp Med. 176: 1191-1195 (1992) and Shopes, B J. Immunol. 148:2918-2922 (1992). To increase a serum half-life of an immunoglobulin, a salvage receptor binding epitope can be incorporated into an immunoglobulin (especially an immunoglobulin fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

A DVD immunoglobulin molecule in accordance with aspects of the invention includes a first variable domain that provides antigen binding functionality, and a second variable domain from a 38C2 antibody, which includes a single, uniquely reactive lysine residue that can be conjugated to a linker.

In certain aspects, the DVD immunoglobulin molecule is bispecific, in that one arm of the immunoglobulin includes a first variable domain with binding specificity for a first binding target, and the second arm includes a first variable domain with binding specificity for a second binding target. Such aspects provide the ability to bind to two different targets, thereby providing additional functionality.

In certain aspects, the DVD immunoglobulin molecule is bi-paratopic, in that one arm of the immunoglobulin includes a first variable domain with binding specificity for a first binding target, and the second arm includes a first variable domain with binding specificity for the same binding target, but a different binding epitope. Such aspects provide the ability to bind to the same target covering two different, but potentially somewhat overlapping binding epitopes, thereby providing target crosslinking functionality, triggering lysosomal trafficking after internalization.

In some embodiments, the immunoglobulin molecule comprises an additional chain. For example, the immunoglobulin molecule comprises a first heavy chain, a second heavy chain and a light chain. The first heavy chain comprises a first variable domain and a second variable domain. The second heavy chain comprises a first variable domain and a second variable domain, and the light chain comprises a variable domain and a constant domain. The first heavy chain and the second heavy chain are capable of forming a heterodimer.

In some embodiments, the organization of the variable and constant domains along a light chain can be organized such that the sequence of the domains along the light chain proceeds from N- to C-terminus as V_{L}1-C_{H}1, the organization of the variable and constant domains along a first heavy chain can be organized such that the sequence of the domains along a first heavy chain proceeds from N- to C-terminus as V_{H}1-V_{H}2-C_{H}1, V_{H}1-C_{H}1-V_{H}2-C_{H}1, V_{H}1-V_{H}2-C_{H}1-FC or V_{H}1-C_{H}1-V_{H}2-C_{H}1-FC, and the organization of the variable and constant domains along a second heavy chain can be organized such that the sequence of the domains along a second heavy chain proceeds from N- to C-terminus as V_{H}1′-V_{H}2′, V_{H}1′-V_{H}2′-C_{H}1′, V_{H}1′-C_{H}1′-V_{H}2′-C_{H}1′, V_{H}1′-V_{H}2′-FC′, V_{H}1′-V_{H}1′-C_{H}1′-FC′ or V_{H}1′-C_{H}1′-V_{H}2′-C_{H}2′-FC′.

It is noted that the variable domains of the first and second heavy chain can bind to the same epitope or different epitopes on the same antigen. For example, the DVD-Ig described herein is a multispecific, e.g., bispecific DVD-Ig, where the variable domains of the first and second heavy chain can bind to different epitopes, where the different epitopes can be on the same antigen or on different antigens. Techniques for making multispecific Ig molecules include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al, EMBO J, 10 (1991) 3655-3659), and “knob-in-hole” engineering (see, e.g., U.S. Pat. No. 5,731,168). Multi-specific antibodies can also be made by engineering electrostatic steering effects for making antibody Fc-heterodimeric molecules (WO 2009/089004): cross-inking two or more antibodies or fragments (see, e.g., U.S. Pat. No. 4,676,980, and Brennan, M. et al, Science 229 (1985) 81-83); using leucine zippers to produce bispecific antibodies (see, e.g., Kostelny, S A et al, J Immunol. 148 (1992) 1547-1553; using “diabody” technology for making bispecific antibody fragments (see, e.g., HoUiger, P. et al, Proc Natl. Acad. Sci. USA 90 (1993) 6444-6448); and using single-chain Fv (scFv) diners (see, e.g. Gruber, M et al, J. Immunol. 152 (1994) 5368-5374); and preparing trispecific antibodies as described, e.g., in Tutt, A, et al, J. Immunol. 147 (1991) 60-69), contents of all of which are incorporated herein by reference in their entireties. Brinkmann and al, in “The making of bispecific antibodies,” (MABS; 2017, VOL 9, NO. 2, 182-212), content of which is incorporated herein by reference in its entirety, focuses on tie various formats and strategies available to generate recombinant bispecific antibodies.

In some embodiments, the first or the second heavy chain comprises a modification in the CH3 domain to reduce the ability of the domain to interact with itself, i.e., form homodimers. In particular, one or more residues that make up the CH3-CH3 interface are replaced with a charged amino acid such that the interaction becomes electrostatically unfavorable. For example, a positive-charged amino acid in the interface, such as a lysine, arginine, or histidine, is replaced with a negative charged amino acid, such as aspartic acid or glutamic acid. In other embodiments, a negative-charged amino acid in the interface is replaced with a positive-charged amino acid. In some embodiments, the amino acid is replaced with an unnatural amino acid having the desired charge characteristic. In some embodiments, the first and the second heavy chain comprise a modification in the CH3 domains to reduce the ability of each CH3 domain to interact with itself but to increase the ability of the domains to interact with each other, i.e., form heterodimers. This can be achieved by replacing one or more residues that make up the CH3-CH3 interface in both CH3 domains with a charged amino acid such that homodimer formation is electrostatically unfavorable but heterodimerization is electrostatically favorable. In certain embodiments, a charged amino acid in each CH3 domain is replaced with an amino acid with an opposite charge. For example, a positive-charged amino acid may be replaced With a negative charged amino acid in the first CH3 domain and a negative charged amino acid may be replaced with a positive-charged amino acid in the second CH3 domain. By reversing the charge of the amino acid, homodimer formation is reduced. When the replacements are coordinated properly, the reversed charges are electrostatically favorable, i.e., opposing charges in the interface, for heterodimerization formation. Some exemplary mutations for enhancing heterodimer formation are listed in Table 1.

TABLE 1
List of some possible pair-wise charge residue
mutations to enhance heterodimer formation^{a}

| | Mutation in | Interacting | Corresponding |
|---|---|---|---|
| Position in | the first | position in | mutation in |
| first CH3 | CH3 domain | second CH3 | the second CH3 |
| Lys409 | Asp or Glu | Asp399′ | Lys or Arg^{b} |
| Lys392 | Asp or Glu | Asp399′ | Lys or Arg^{b} |
| Lys439 | Asp or Glu | Asp356′ | Lys or Arg^{b} |
| Lys370 | Asp or Glu | Glu357 | Lys or Arg |
| Asp399 | Lys or Arg | Lys409 | Asp or Glu |
| Asp399 | Lys or Arg | Lys392 | Asp or Glu |
| Asp356 | Lys or Arg | Lys439 | Asp or Glu |
| Glu357 | Lys or Arg | Lys370′ | Asp or Glu |
| Arg355 | Asp or Glu | Arg355^{c} | Asp or Glu |
| Lys 360 | Asp or Glu | Lys360^{c} | Asp or Glu |

^{a}Combinations of the above pair-wise charge residue mutations could also be used. For example, Lys409 - - - Asp399′ interaction pair mutations could be combined with Lys439 - - - Asp356′ pair mutations.
^{b}Histidine (His) could also be added to this list of positively charged residues, however, increase in side chain volume and pH dependency should be taken into account in the design.
^{c}These single residue mutations could be combined with other pair-wise mutations listed in the table to enhance the heterodimer formation.

In some embodiments, the first CH3 domain comprises the amino acid modifications L351Y, F405A, and Y407V, and the second CH3 domain comprises the amino acid modifications T366L, K392M, and T394W. In some embodiments, the first CH3 domain comprises the amino acid modifications L351Y, F405A, and Y407V, and the second CH3 domain comprises the amino acid modifications T366L, K392L, and T394W In some embodiments, the first CH3 domain comprises the amino acid modifications T350V, L351Y, F4105A, and Y4107V, and the second CH3 domain comprises the amino acid modifications T350V, T3661L, K392M, and T394W, In some embodiments, the first C1H3 domain comprises the amino acid modifications T350V, L351Y, F405A, and Y407V, and the second CH3 domain comprises the amino acid modifications T350V, T366L, K392L, and T394W. In some embodiments, the first CH3 domain comprises the amino acid modifications T366L, N390R, K392R, and T394W, and the second CH3 domain comprises the amino acid modifications L351Y, S400E, F405A, and Y107V. In some embodiments, the first CH3 domain comprises the amino acid modifications T350V, T366L, N390R, K392R, and T394W, and the second (13 domain comprises the amino acid modifications T350V, L351Y, S400E, F405A, and Y407V.

In some embodiments, one of the CH3 domains comprises one or more, e.g., 1, 2, 3, 4, 5, 6, 7, 8, 9, 10 or more of the following modifications K392D, K392E, N392D, N392E, R409D, R409E, K409D, K409E, D399K, D399R, E356R, E356K, D356R, D356K, Y349T, L351T, L368T, L398T, F405T, Y407T, Y407R, L234A and L235A.

In some embodiments, the first CH3 domain comprises the amino acid modifications S345C and T366W, and the second CH3 domain comprises the amino acid modifications Y349C, T366S, L368A and Y407V.

In certain aspects, an immunoglobulin molecule is an intact immunoglobulin molecule that includes a first and second variable region, as described above, and also includes a C_{L} domain on the light chain, as well as heavy chain constant domains C_{H}1, C_{H}2, and C_{H}3. A constant domain can comprise a native or non-native sequence, or an amino acid sequence variant thereof. In certain aspects, an immunoglobulin molecule can be an immunoglobulin fragment. Examples of immunoglobulin fragments include, but are not limited to, (Fab′)₂, Fab′, Fab, and Fv fragments.

### Ligands

A wide variety of entities can be coupled to the DVD immunoglobulin described herein. Preferred moieties are ligands, which are coupled, preferably covalently, either directly or indirectly via a linker to the DVD immunoglobulin described herein. Ligands can include a naturally occurring substance, such as peptides, polypeptides, a carbohydrate (e.g., a dextran, pullulan, chitin, chitosan, inulin, cyclodextrin or hyaluronic acid), a vitamin, or a lipid. The ligand can also be a recombinant or synthetic molecule, such as a synthetic polymer.

Generally, a ligand alters the distribution, targeting or lifetime of the DVD immunoglobulin into which it is incorporated. Some ligands can have endosomolytic properties. Such ligands are also referred to as endosomolytic ligands herein. The endosomolytic ligands promote the lysis of the endosome and/or transport of the composition of the invention, or its components, from the endosome to the cytoplasm of the cell. Endosomolytic ligands include, but are not limited to, imidazoles, poly or oligoimidazoles, PEIs, peptides, fusogenic peptides, polycarboxylates, polycations, masked oligo or poly cations or anions, acetals, polyacetals, ketals/polyketals, orthoesters, polymers with masked or unmasked cationic or anionic charges, dendrimers with masked or unmasked cationic or anionic charges.

The endosomolytic ligand can be a polyanionic peptide or peptidomimetic which shows pH-dependent membrane activity and fusogenicity. In some embodiments, the endosomolytic ligand assumes its active conformation at endosomal pH. The “active” conformation is that conformation in which the endosomolytic ligand promotes lysis of the endosome and/or transport of the composition of the invention, or its components, from the endosome to the cytoplasm of the cell. Exemplary endosomolytic ligands include the GALA peptide (Subbarao et al., Biochemistry, 1987, 26: 2964-2972, which is incorporated by reference in its entirety), the EALA peptide (Vogel et al., J. Am. Chem. Soc., 1996, 118: 1581-1586, which is incorporated by reference in its entirety), and their derivatives (Turk et al., Biochem. Biophys. Acta, 2002, 1559: 56-68, which is incorporated by reference in its entirety). In some embodiments, the endosomolytic ligand can contain a chemical group (e.g., an amino acid) which will undergo a change in charge or protonation in response to a change in pH. The endosomolytic ligand can be linear or branched.

In some embodiment, the endosomolytic ligand is a peptide comprising an amino acid sequence FSEAIKKIIDFLG (SEQ ID NO: 17).

It is noted that the ligand, e.g., endosomolytic ligand can be attached to the heavy chain, light chain or the double-stranded RNA molecule of a DVD-Ig described herein. For example, the ligand, e.g., endosomolytic ligand can be attached to the N- or C-terminus of the light chain or the heavy chain. In some embodiments, the ligand, e.g., endosomolytic ligand is attached to the C-terminus of the light chain.

### Exemplary DVD Immunoglobulins

In some embodiments, the DVD immunoglobulin includes a first variable domain that binds to CD138. For example, the DVD immunoglobulin comprises a first variable domain that binds to CD138 and a humanized 38C2 antibody variable domain as the second variable domain. The variable domains are connected on each light and heavy chain with a peptide linker sequence, e.g., selected from ASTKGP (SEQ ID NO: I), TVAAPSVFIFPP (SEQ ID NO: 2), GIS (SEQ ID NO: 3), (G₄S)₂ (SEQ ID NO: 4), (G₄S)₃ (SEQ ID NO: 5), EPKSCDG₄S (SEQ ID NO:6), EPKSCD(G₄S)₂ (SEQ ID NO: 7), and EPKSCD(G₄S)₃ (SEQ ID NO: 8). For example, the DVD immunoglobulin comprises:
(i) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 18)
MPMGSLQPLATLYLLGMLVASVLADIQMTQSTSSLSASLGDRVTISCSAS
QGINNYLNWYQQKPDGTVELLIYYTSTLQSGVPSRFSGSGSGTDYSLTIS
NLEPEDIGTYYCQQYSKLPRTFGGGTKLEIKASTKGPELQMTQSPSSLSA
SVGDRVTITCRSSQSLLHTYGSPYLNWYLQKPGQSPKLLIYKVSNRFSGV
PSRFSGSGSGTDFTLTISSLQPEDFAVYFCSQGTHLPYTFGGGTKVEIKR
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC;

and/or
(ii) a heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 19)
MPMGSLQPLATLYLLGMLVASVLAQVQLQQSGSELMMPGASVKISCKATG
YTFSNYWIEWVKQRPGHGLEWIGEILPGTGRTIYNEKFKGKATFTADISS
NTVQMQLSSLTSEDSAVYYCARRDYYGNFYYAMDYWGQGTSVTVSSASTK
GPEVQLVESGGGLVQPGGSLRLSCAASGFTFSNYWMSWVRQSPEKGLEWV
SEIRLRSDNYATHYAESVKGRFTISRDNSKNTLYLQMNSLRAEDTGIYYC
KTYFYSFSYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
A.

Additional exemplary DVD immunoglobulins comprising a first variable domain that binds to CD138 are described in WO2017/049139, content of which is incorporated herein by reference in its entirety.

In some embodiments, the DVD immunoglobulin includes a first variable domain that binds to BCMA. For example, the DVD immunoglobulin comprises a first variable domain that binds to BCMA and a humanized 38C2 antibody variable domain as the second variable domain. The variable domains are connected on each light and heavy chain with a peptide linker sequence, e.g., selected from ASTKGP (SEQ ID NO: I), TVAAPSVFIFPP (SEQ ID NO: 2), G₄S (SEQ ID NO: 3), (G₄S)₂ (SEQ ID NO: 4), (G₄S)₃ (SEQ ID NO: 5), EPKSCDG₄S (SEQ ID NO:6), EPKSCD(G₄S)₂ (SEQ ID NO: 7), and EPKSCD(G₄S)₃ (SEQ ID NO: 8). For example, the DVD immunoglobulin comprises:
(i) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 20)
MPMGSLQPLATLYLLGMLVASVLADIQMTQSPSSLSASVGDRVTITCSAS
QDISNYLNWYQQKPGKAPKLLIYYTSNLHSGVPSRFSGSGSGTDFTLTIS
SLQPEDFATYYCQQYRKLPWTFGQGTKLEIKASTKGPELQMTQSPSSLSA
SVGDRVTITCRSSQSLLHTYGSPYLNWYLQKPGQSPKLLIYKVSNRFSGV
PSRFSGSGSGTDFTLTISSLQPEDFAVYFCSQGTHLPYTFGGGTKVEIKR
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC;

and/or
(ii) a heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 21)
MPMGSLQPLATLYLLGMLVASVLAQVQLVQSGAEVKKPGSSVKVSCKA
SGGTFSNYWMHWVRQAPGQGLEWMGATYRGHSDTYYNQKFKGRVTIT
ADKSTSTAYMELSSLRSEDTAVYYCARGAIYDGYDVLDNWGQGTLVTV
SSASTKGPEVQLVESGGGLVQPGGSLRLSCAASGFTFSNYWMSWVRQSP
EKGLEWVSEIRLRSDNYATHYAESVKGRFTISRDNSKNTLYLQMNSLRA
EDTGIYYCKTYFYSFSYWGQGTLVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEAL
HNHYTQKSLSLSPGA.

In some embodiments, the DVD immunoglobulin includes a first variable domain that binds to SLAMF7. For example, the DVD immunoglobulin comprises a first variable domain that binds to SLAMF7 and a humanized 38C2 antibody variable domain as the second variable domain. The variable domains are connected on each light and heavy chain with a peptide linker sequence, e.g., selected from ASTKGP (SEQ ID NO: I), TVAAPSVFIFPP (SEQ ID NO: 2), G₄S (SEQ ID NO: 3), (G₄S)₂ (SEQ ID NO: 4), (G₄S)₃ (SEQ ID NO: 5), EPKSCDG₄S (SEQ ID NO:6), EPKSCD(G₄S)₂ (SEQ ID NO: 7), and EPKSCD(G₄S)₃ (SEQ ID NO: 8). For example, the DVD immunoglobulin comprises:
(i) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 22)
MPMGSLQPLATLYLLGMLVASVLADIQMTQSPSSLSASVGDRVTITCKA
SQDVGIAVAWYQQKPGKVPKLLIYWASTRHTGVPDRFSGSGSGTDFTLT
ISSLQPEDVATYYCQQYSSYPYTFGQGTKVEIKASTKGPELQMTQSPSS
LSASVGDRVTITCRSSQSLLHTYGSPYLNWYLQKPGQSPKLLIYKVSNR
FSGVPSRFSGSGSGTDFTLTISSLQPEDFAVYFCSQGTHLPYTFGGGTK
VEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGL
SSPVTKSFNRGEC;

and/or
(ii) a heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 23)
MPMGSLQPLATLYLLGMLVASVLAEVQLVESGGGLVQPGGSLRLSCAAS
GFDFSRYWMSWVRQAPGKGLEWIGEINPDSSTINYAPSLKDKFIISRDN
AKNSLYLQMNSLRAEDTAVYYCARPDGNYWYFDVWGQGTLVTVSSASTK
GPEVQLVESGGGLVQPGGSLRLSCAASGFTFSNYWMSWVRQSPEKGLEW
VSEIRLRSDNYATHYAESVKGRFTISRDNSKNTLYLQMNSLRAEDTGIY
YCKTYFYSFSYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGA.

In some embodiments, the DVD immunoglobulin includes a first variable domain that binds to HER2. For example, the DVD immunoglobulin comprises a first variable domain that binds to HER2 and a humanized 38C2 antibody variable domain as the second variable domain. The variable domains are connected on each light and heavy chain with a peptide linker sequence, e.g., selected from ASTKGP (SEQ ID NO: I), TVAAPSVFIFPP (SEQ ID NO: 2), GIS (SEQ ID NO: 3), (G₄S)₂ (SEQ ID NO: 4), (G₄S)₃ (SEQ ID NO: 5), EPKSCDG₄S (SEQ ID NO:6), EPKSCD(G₄S)₂ (SEQ ID NO: 7), and EPKSCD(G₄S)₃ (SEQ ID NO: 8). For example, the DVD immunoglobulin comprises:
(i) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 24)
MDWTWRILFLVAAATGAHSDIQMTQSPSSLSASVGDRVTITCRASQDVN
TAVAWYQQKPGKAPKLLIYSASFLYSGVPSRFSGSRSGTDFTLTISSLQ
PEDFATYYCQQHYTTPPTFGQGTKVEIKASTKGPELQMTQSPSSLSASV
GDRVTITCRSSQSLLHTYGSPYLNWYLQKPGQSPKLLIYKVSNRFSGVP
SRFSGSGSGTDFTLTISSLQPEDFAVYFCSQGTHLPYTFGGGTKVEIKR
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC;

and/or
(ii) a heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 25)
MDWTWRILFLVAAATGAHSEVQLVESGGGLVQPGGSLRLSCAASGFNIK
DTYIHWVRQAPGKGLEWVARIYPTNGYTRYADSVKGRFTISADTSKNTA
YLQMNSLRAEDTAVYYCSRWGGDGFYAMDYWGQGTLVTVSSASTKGP
EVQLVESGGGLVQPGGSLRLSCAASGFTFSNYWMSWVRQSPEKGLEWVS
EIRLRSDNYATHYAESVKGRFTISRDNSKNTLYLQMNSLRAEDTGIYYC
KTYFYSFSYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGA.

Additional, exemplary DVD immunoglobulins comprising a first variable domain that binds to HER2, FOLR1 or CD79b are described in WO2017/049139, content which is incorporated herein by reference in its entirety.

In some embodiments, the DVD immunoglobulin includes a first variable domain that binds to SLAMF7, and the DVD immunoglobulin further includes a endosomolytic ligand. For example, the DVD immunoglobulin comprises a first variable domain that binds to SLAMF7 and a humanized 38C2 antibody variable domain as the second variable domain. The variable domains are connected on each light and heavy chain with a peptide linker sequence, e.g., selected from ASTKGP (SEQ ID NO: 1), TVAAPSVFIFPP (SEQ ID NO: 2), G₄S, (G₄S)₂, (G₄S)₃, EPKSCDG₄S, EPKSCD(G₄S)₂, and EPKSCD(G₄S)₃. For example, the DVD immunoglobulin comprises:
(i) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 26)
MPMGSLQPLATLYLLGMLVADQQLTQSPSSLSASVGDRVTITCRASQSI
GSWLSWYQQKPGKAPKLLIYGASNLASGVPSRFSGSRSGTDYTLTISSL
QPEDFATYYCLGASPNGWAFGQGTKVEIKASTKGPELQMTQSPSSLSAS
VGDRVTITCRSSQSLLHTYGSPYLNWYLQKPGQSPKLLIYKVSNRFSGV
PSRFSGSGSGTDFTLTISSLQPEDFAVYFCSQGTHLPYTFGGGTKVEIK
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGECFSEAIKKIIDFLG;

and/or
(ii) a heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 27)
MPMGSLQPLATLYLLGMLVAEQQVVESGGGLVQPGGSLRLSCAVSGFSL
NSYGVIWVRQAPGKGLEYVSIIGSSGNTYYASSVKGRFTISRDTRLNTV
YLQMNSLRAEDTAVYFCARYYGDSGFDSWGQGTLVTVSSASTKGPEVQL
VESGGGLVQPGGSLRLSCAASGFTFSNYWMSWVRQSPEKGLEWVSEIRL
RSDNYATHYAESVKGRFTISRDNSKNTLYLQMNSLRAEDTGIYYCKTYF
YSFSYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGA.

In some embodiments, the DVD immunoglobulin comprises:
(i) a first heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 28)
MDWTWRILFLVAAATGAHSDVVMTQTPSSVPAAVGGTVTINCQASQSID
SNLAWFQQKPGQPPNLLIYDASTLASGVPSRFKGSGAGKQFTLTISGVQ
REDAATYYCLGSYSRTEKAFGAGTKVEIKGGGGSGGGGSGGGGSQEQLE
ESGGRLVTPGTPLTLTCTVSGFSLSNYHMSWVRQAPGKGLEWIGFITSG
GSTYYASWAKGRFTISRTSTTVDLKITSPTTEDTATYFCARWNGYGGNM
WGPGTLVTVEPKSSDKTHTCPPCPAPELLGGPSVFLFPPKPKDTLMISR
TPEVTCVVVDVSHEDPEVKFNWYVDGVEVHNAKTKPREEQYASTYRVVS
VLTVLHQDWLNGKEYKCKVSNKALPAPIEKTISKAKGQPREPQVCTLPP
SRDELTKNQVSLSCAVKGFYPSDIAVEWESNGQPENNYKTTPPVLDSDG
SFFLVSKLTVDKSRWQQGNVFSCSVMHEALHNHYTQKSLSLSPGA;

and/or
(ii) a second heavy chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 29)
MDWTWRILFLVAAATGAHSEVQLVESGGGLVQPGGSLRLSCAASGFTFS
NYWMSWVRQSPEKGLEWVSEIRLRSDNYATHYAESVKGRFTISRDNSKN
TLYLQMNSLRAEDTGIYYCKTYFYSFSYWGQGTLVTVSSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDGGGG
SGGGGSEVQLVESGGGLVQPGGSLRLSCAASGFTFSNYWMSWVRQSPEK
GLEWVSEIRLRSDNYATHYAESVKGRFTISRDNSKNTLYLQMNSLRAED
TGIYYCKTYFYSFSYWGQGTLVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKRVEPKSSDKTHTCPPCPAPELLGGP
SVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTI
SKAKGQPREPQVYTLPPCRDELTKNQVSLWCLVKGFYPSDIAVEWESNG
QPENNYKTTPPVLDSDGSFFLYSKLTVDKSRWQQGNVFSCSVMHEALHN
HYTQKSLSLSPGA;

and/or
(iii) a light chain comprising an amino acid sequence that has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity or is substantially similar to the amino acid sequence:

(SEQ ID NO: 30)
MDWTWRILFLVAAATGAHSELQMTQSPSSLSASVGDRVTITCRSSQSLLH
TYGSPYLNWYLQKPGQSPKLLIYKVSNRFSGVPSRFSGSGSGTDFTLTIS
SLQPEDFAVYFCSQGTHLPYTFGGGTKVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

### Production of DVD Immunoglobulins

DVD immunoglobulins of the present invention can be produced by any of a number of techniques known in the art. For example, expression from host cells, wherein expression vector(s) encoding the DVD heavy and/or DVD light chains is transfected into a host cell by standard techniques. Various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD immunoglobulins of the invention in either prokaryotic or eukaryotic host cells, expression of DVD immunoglobulins in eukaryotic cells is preferable, and most preferable in mammalian host cells, because such eukaryotic cells (and in particular mammalian cells) are more likely than prokaryotic cells to assemble and secrete a properly folded and immunologically active DVD immunoglobulin.

Preferred mammalian host cells for expressing the recombinant immunoglobulins of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), Human Embryonic Kidney (HEK) cells, NS0 myeloma cells, COS cells and SP2 cells. When recombinant expression vectors encoding DVD immunoglobulins are introduced into mammalian host cells, the DVD immunoglobulins are produced by culturing the host cells for a period of time sufficient to allow for expression of the DVD immunoglobulins in the host cells or, more preferably, secretion of the DVD immunoglobulins into the culture medium in which the host cells are grown. DVD immunoglobulins can be recovered from the culture medium using standard protein purification methods.

In a preferred system for recombinant expression of DT) immunoglobulins of the invention, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. A recombinant expression vector also carries a DHFR gene, which allows for selection of CHO cells that have been transfected with the vector using methotrexate selection/amplification. Selected transformant host cells are cultured to allow for expression of the DVD heavy and light chains and intact DVD immunoglobulin is recovered from the culture medium. Standard molecular biology and tissue culture techniques are used to prepare the recombinant expression vector, transfect the host cells, select for transformants, culture the host cells and recover the DVD immunoglobulin from the culture medium. In addition, aspects of the invention include a method of synthesizing a DVD immunoglobulin of the invention by culturing a host cell of the invention in a suitable culture medium until a DVD immunoglobulin of the invention is synthesized. A method can further comprise isolating the DVD immunoglobulin from the culture medium to yield an isolated immunoglobulin.

A feature of the subject DVD immunoglobulins is that they can be produced and purified in ways that are similar to conventional antibodies. Production of DVD immunoglobulins can result in a homogeneous, single major product with desired activity, without any sequence modification of the constant region or chemical modifications of any kind.

### Linkers

Aspects of the conjugates disclosed herein include linkers, which can comprise one or more linker components. The term “linker” means an organic moiety that connects two parts of a compound, e.g., a DVD immunoglobulin to a dsRNA. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

Without limitations, various types of linker functionality can be included in the subject conjugates, including but not limited to cleavable linkers, and non-cleavable linkers, as well as reversible linkers and irreversible linkers.

In some embodiments, the linker is a cleavable linker. Cleavable linkers are those that rely on processes inside a target cell to liberate the two parts the linker is holding together, e.g., the DVD-Ig and the dsRNA, as reduction in the cytoplasm, exposure to acidic conditions in a lysosome or endosome, or cleavage by specific enzymes (e.g. proteases) within the cell. As such, cleavable linkers allow the two dsRNA to be released in its original form after the conjugate has been internalized and processed inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the dsRNA molecule according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the dsRNA molecule according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the dsRNA molecule according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the dsRNA molecule according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCR^{B}C(O)—, where R^{A} and R^{B} are the R groups of the two adjacent amino acids. These candidates can be evaluated using methods analogous to those described above.

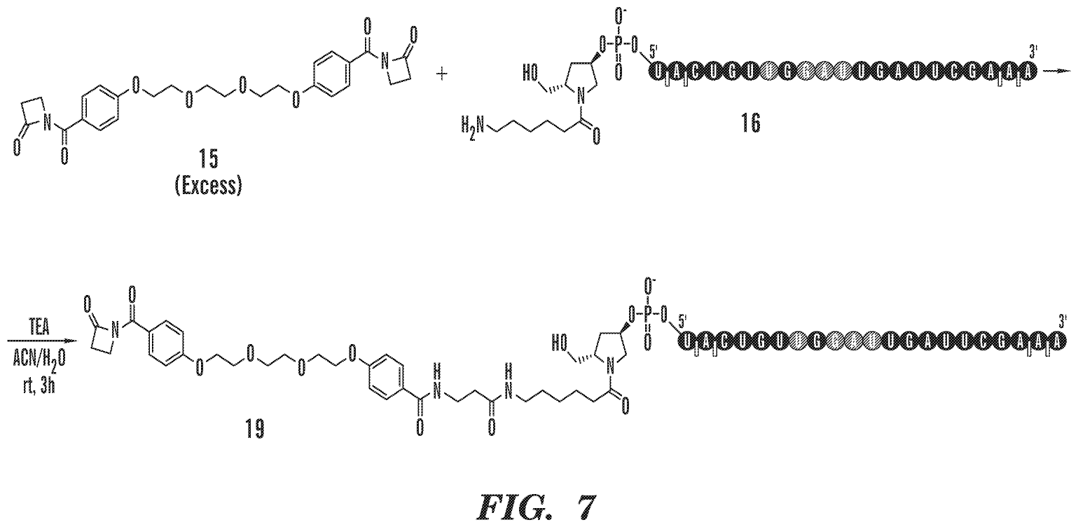

Non-limiting examples of cleavable linkers are described in FIG. 7 of WO2017/049139, content of which is incorporated herein by reference in its entirety.

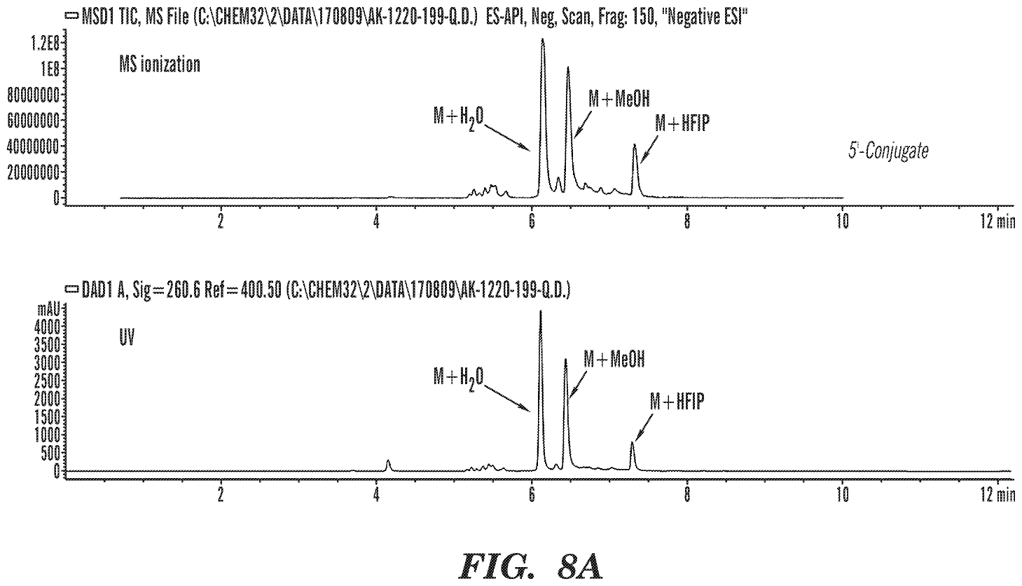

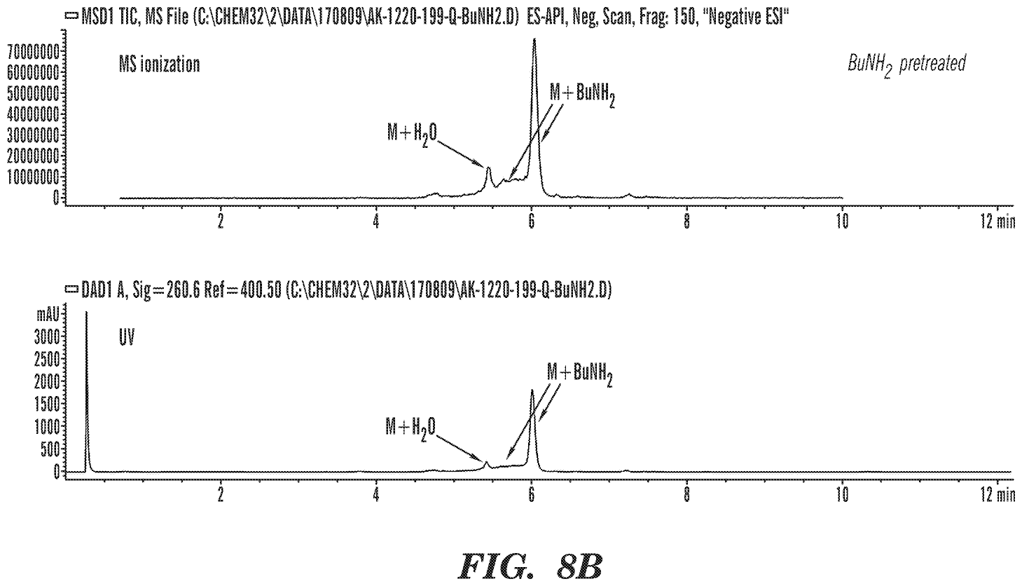

Non-cleavable linkers utilize catabolic degradation of an immunoconjugate for the release of the drug moiety. A released drug moiety generally retains the linker as well as the amino acid residue of the immunoglobulin to which the linker was conjugated. Non-cleavable linkers include, but are not limited to, PEG linkers, hydrocarbon linkers, and thioether linkers. Non-limiting examples of non-cleavable linkers are described in FIG. 8 of WO2017/049139, content of which is incorporated herein by reference in its entirety.

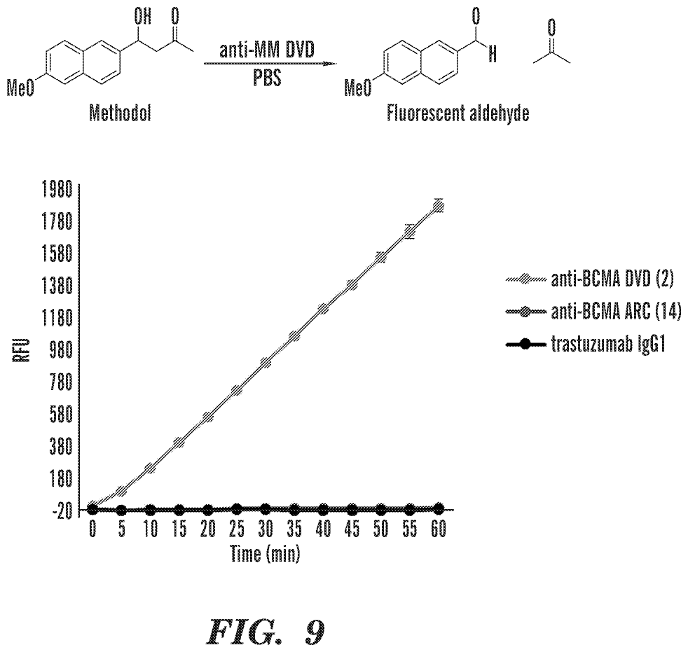

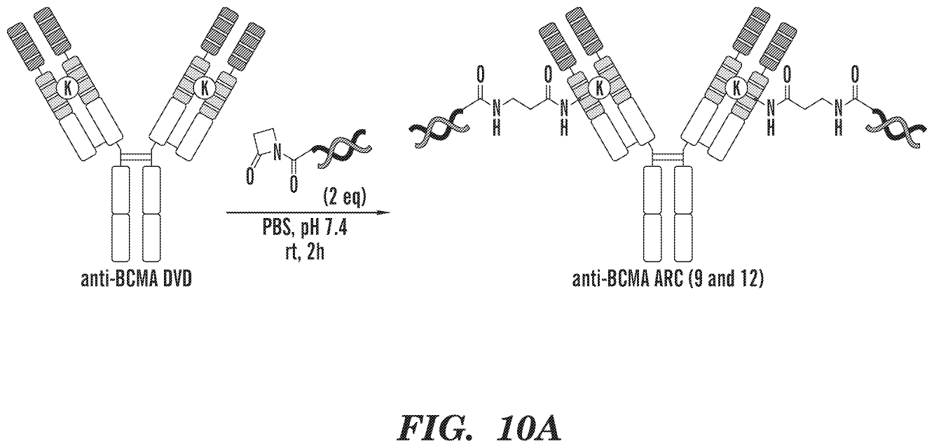

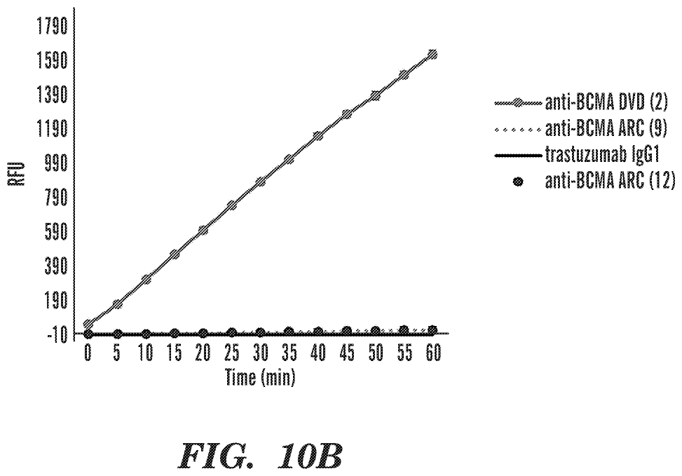

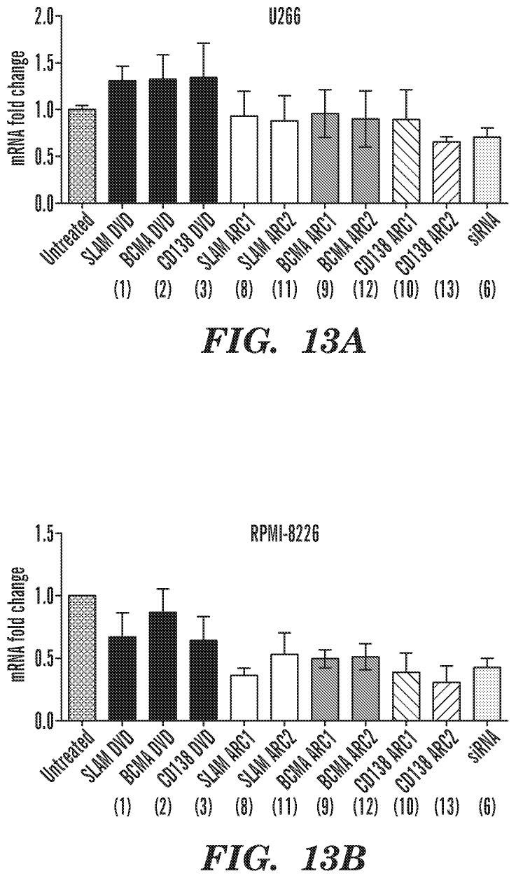

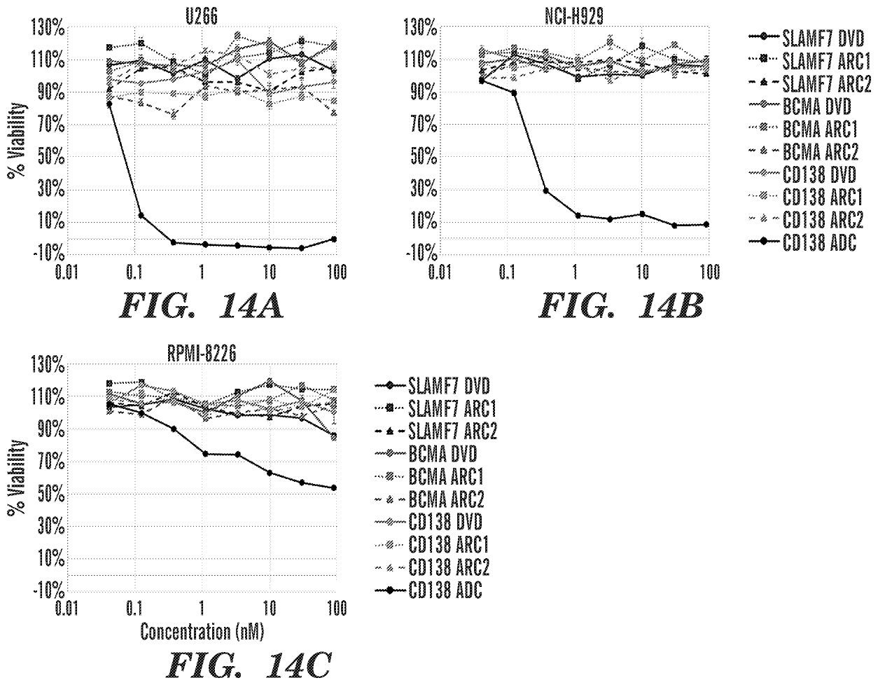

Aspects of the subject conjugates can also include reversible and irreversible linkers. Reversible linkers utilize chemical bonds that can readily be broken, or reversed, using suitable reagents. As such, after the formation of a reversible linker, the linker can be broken in a desired position by treatment with a reagent, thereby releasing the immunoglobulin molecule from the linker. Non-limiting examples of reversible linkers are described in FIG. 9 of WO2017/049139, content of which is incorporated herein by reference in its entirety. Irreversible linkers utilize chemical bonds that cannot readily be broken or reversed after their formation. As such, after the formation of an irreversible linker, an immunoglobulin molecule cannot readily be released. Non-limiting examples of irreversible linkers are provided are described in FIG. 10 of WO2017/049139, content of which is incorporated herein by reference in its entirety. Example linker reactions in which an immunoglobulin is conjugated to a reversible or irreversible linker are described in FIG. 13 of WO2017/049139, content of which is incorporated herein by reference in its entirety. In addition to β-lactam and diketone moieties, in some embodiments, other moieties, such as, e.g., vinyl diketones and pro-vinyl diketones can be used for conjugation. In some embodiments, electrophilic moieties (handles) can be used, either alone or in combination, with such moieties. Electrophilic moieties can be used for site-specific conjugation with the single, uniquely reactive lysine of an h38C2 variable domain, and can also be used for non-specific conjugation after an h38C2 lysine has been conjugated to a drug moiety. Non-limiting examples of other moieties include 6-maleimidocaproyl (“MC”), maleimidopropanoyl (“MIP”), valine-citrulline (“val-cit” or “vc”), alanine-phenylalanine (“ala-phe”), p-aminobenzyloxycarbonyl (a “PAB”), and those resulting from conjugation with linker reagents: N-Succinimidyl 4-(2-pyridylthio) pentanoate forming linker moiety 4-mercaptopentanoic acid (“SPP”), N-succinimidyl 4-(N-maleimidom ethyl) cyclohexane-1 carboxylate forming linker moiety 4-((2,5-dioxopyrrolidin-1-yl)methyl)cyclohexanecarboxylic acid (“SMCC”, also referred to herein as “MCC”), 2,5-dioxopyrrolidin-1-yl 4-(pyridin-2-yldisulfanyl) butanoate forming linker moiety 4-mercaptobutanoic acid (“SPDB”), N-Succinimidyl (4-iodo-acetyl) aminobenzoate (“SIAB”), ethyleneoxy —CH₂CH₂O— as one or more repeating units (“EO” or “PEO”). Further information is provided in Sinha et al., Nat. Protoc. 2, 449-456 (2007), the disclosure of which is incorporated by reference herein in its entirety.

In some embodiments, a linker component can comprise an amino acid unit. In one such aspect, an amino acid unit allows for cleavage of the linker by a protease, thereby facilitating release of the drug from the immunoconjugate upon exposure to intracellular proteases, such as lysosomal enzymes. See, e.g., Doronina et al. (2003) Nat. Biotechnol. 21:778-784. Non-limiting examples of amino acid units include, but are not limited to, a dipeptide, a tripeptide, a tetrapeptide, and a pentapeptide. Non-limiting examples of dipeptides include: valine-citrulline (vc or val-cit), alanine-phenylalanine (af or ala-phe); phenylalanine-lysine (fk or phe-lys); or N-methyl-valine-citrulline (Me-val-cit). Non-limiting examples of tripeptides include: glycine-valine-citrulline (gly-val-cit) and glycine-glycine-glycine (gly-gly-gly). An amino acid unit can comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, a linker L can be a branched or dendritic type linker for covalent attachment of more than dsRNA through a branching, multifunctional linker moiety to an immunoglobulin (Sun et al (2002) Bioorganic & Medicinal Chemistry Letters 12:2213-2215; Sun et al (2003) Bioorganic & Medicinal Chemistry 11: 1761-1768). Non-limiting examples of branched, dendritic linkers include 2,6-bis(hydroxymethyl)-p-cresol and 2,4,6-tris(hydroxymethyl)-phenol dendrimer units (WO 2004/01993; Szalai et al (2003) J. Amer. Chem. Soc. 125: 15688-15689; Shamis et al (2004) J. Amer. Chem. Soc. 126: 1726-1731; Amir et al (2003) Angew. Chem. Int. Ed. 42:4494-4499). Branched linkers can increase the molar ratio of RNA to immunoglobulin, i.e., loading, which is related to the potency of the ADC. Thus, for example, where an immunoglobulin bears only one reactive amino acid residue for conjugation, a multitude of dsRNAs can be attached through a branched linker. Without limitations, the branch-point of the branched linker can be at least trivalent, but can be a tetravalent, pentavalent or hexavalent atom, or a group presenting such multiple valencies. In certain embodiments, the branch-point can be —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branch-point can be glycerol or a glycerol derivative.

Linker components, including stretcher, spacer, and amino acid units, can be synthesized by methods known in the art, such as those described in US Patent Publication No. 2005/0238649 A1, which is herein incorporated by reference in its entirety.

In some embodiments, the linker is of structure:

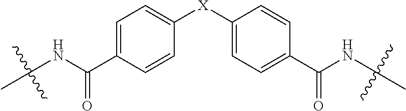

wherein X is a spacer.

Spacer X typically comprises a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, spacer X is —O(CH₂CH₂O)ₚCH₂CH₂O—, where p can be 0 or an integer from 1 to 1000. For example, p can be 0, 1, 2, 3, 4, 5, 6, 7, 8, 9 or 10. In some preferred embodiments, p is 2.

In some embodiments, the spacer X comprises at least one cleavable linking group. For example, spacer X comprises a disulfide, e.g., —SS— linkage. In some embodiments, the embodiments, spacer X is —O(alkyl)-SS—(alkyl)O—, where each alkyl can be interrupted by one, e.g., two, three or more of groups independently selected from O, S, S(O), SO₂, NH, C(O) or C(O)O. For example, spacer X can be —O(CH₂)_{q}O(CH₂)ᵣSS(CH₂O)ₛ(CH₂)ₜO—, where q, r, s and t are independently selected integers from 1-15. For example, each q, r, s, and t can be independently 1, 2, 3, 4, 5, 6, 7 or 8. It is noted that q, r, s, and t can all be same, all same or some same and some different. For example, q and t can be same and selected from 2, 3, 4, 5 and 6. Similarly r and s can be same and selected from 1, 2, 3, and 4. In some embodiments, q and t are same, and r and s are the same but different from q and t. In some preferred embodiments, q and t are 4; and r and s are 2.

### Synthesis of Conjugates

The conjugates described herein can be prepared using any method known in the art for conjugating two molecules together. For example, the conjugates can be prepared using the method disclosed in the Examples section herein. In some embodiments, the second variable domain of DVD-Ig includes a reactive lysine residue, and a conjugate is created using a controlled conjugation reaction wherein a linker/dsRNA molecule composition is conjugated to the reactive lysine residue on each heavy chain of a naked Ig. Conditions for this reaction are described, for example, in U.S. Pat. No. 8,252,902, which is herein incorporated by reference in its entirety. Briefly, the reaction can be carried out at room temperature in a solution of PBS, pH 7.4, 2% DMSO by reacting the Ig with a linker/dsRNA moiety composition, thereby resulting in the attachment of one linker/dsRNA moiety to each of the reactive lysine residues on the Ig. The result is conjugate having two dsRNA moieties attached via linkers to the reactive lysine residues on each heavy chain of the Ig.

In certain aspects, additional dsRNA molecule can be conjugated to the DVD-Ig molecule using uncontrolled conjugation techniques. For example, in certain aspects, amino acid residues other than the single, uniquely reactive lysine residue of the 38C2 variable domain can be used as attachment points for conjugation of a dsRNA molecule via a linker. The result of such uncontrolled conjugation is a conjugate having one or more dsRNA molecules attached to the other amino acid residues on the immunoglobulin molecule. Such additional conjugation can be accomplished by reacting a linker/dsRNA molecule composition with, e.g., lysine residues on the immunoglobulin molecule other than the single, uniquely reactive lysine in the second variable domain, or standard or engineered cysteine residues on the immunoglobulin molecule, or one or more engineered selenocysteine residues on the immunoglobulin molecule, or a uniquely reactive arginine residue in the second variable domain. The result of such uncontrolled conjugation is a conjugate with an average number of dsRNA molecules that ranges from about 1 to about 20 dsRNA molecules per antibody, depending on the number of amino acid residues that are available to react with the linker/dsRNA molecule composition. In certain aspects, the average number of dsRNA molecules per immunoglobulin molecule achieved using an uncontrolled conjugation approach is about 1 to about 8, such as 2, 3, 4, 5, 6, or 7 dsRNA molecules per immunoglobulin.

Generally, an immunoglobulin is composed of two identical light chains and two identical heavy chains. In one aspect, an immunoglobulin light chain comprises a kappa light chain. In one aspect, an immunoglobulin light chain comprises a lambda light chain. In one aspect, an immunoglobulin is an IgA immunoglobulin, having a heavy chain. In one aspect, an immunoglobulin is an IgA1 immunoglobulin. In one aspect, an immunoglobulin is an IgA2 immunoglobulin. In one aspect, an immunoglobulin is an IgD immunoglobulin, having a S heavy chain. In one aspect, an immunoglobulin is an IgE immunoglobulin, having an c heavy chain. In one aspect, an immunoglobulin is an IgG immunoglobulin, having a γ heavy chain. In one aspect, an immunoglobulin is an IgG1 immunoglobulin. In one aspect, an immunoglobulin is an IgG2 immunoglobulin. In one aspect, an immunoglobulin is an IgG3 immunoglobulin. In one aspect, an immunoglobulin is an IgG4 immunoglobulin. In one aspect, an immunoglobulin is an IgM immunoglobulin, having a μ heavy chain.

In one aspect, an immunoglobulin is an intact immunoglobulin. In one aspect, an immunoglobulin is a naked immunoglobulin. In one aspect, an immunoglobulin is an immunoglobulin fragment. In one aspect, an immunoglobulin fragment is selected from the group consisting of Fab, Fab′, F(ab′)₂, Fv and scFv.

In some aspects, an immunoglobulin is a dual variable domain immunoglobulin. In some aspects, an immunoglobulin comprises a native polypeptide sequence. In some aspects, an immunoglobulin comprises a non-native polypeptide sequence. In some aspects, an immunoglobulin comprises a polypeptide. In some aspects, an immunoglobulin is a monoclonal immunoglobulin. In some aspects, an immunoglobulin comprises a chimeric immunoglobulin. In some aspects, an immunoglobulin comprises a humanized immunoglobulin. In some aspects, an immunoglobulin comprises a human immunoglobulin. In some aspects, an immunoglobulin is an isolated immunoglobulin. In some aspects, an immunoglobulin comprises a polypeptide sequence that is a fusion of two or more polypeptide sequences. In some aspects, an immunoglobulin is a conjugated immunoglobulin.

In some aspects, and immunoglobulin specifically binds to or is specific for a binding target. In some aspects, an immunoglobulin has a binding affinity. In some aspects, an immunoglobulin has a K_{d} value. In some aspects, an immunoglobulin binds to an epitope. In some aspects, an immunoglobulin binds to a target or binding target. In some aspects, a binding target comprises a binding region, to which an immunoglobulin binds. In some aspects, an immunoglobulin binds to an antigen. In some aspects, an immunoglobulin comprises an antigen binding site or antigen binding region.

In some aspects, an immunoglobulin is produced in a host cell. In some aspects, an immunoglobulin is produced by a cell line or a cell culture. In some aspects, an immunoglobulin is produced from a nucleic acid sequence that is operably linked to another nucleic acid sequence.

In some aspects, an immunoglobulin amino acid sequence has a percent amino acid sequence identity to another amino acid sequence.

### Uses of the Conjugates

The conjugates described herein can be used for inhibiting the expression of a target gene. Accordingly, in another aspect, provided herein is a method for inhibiting the expression of a target gene. The method comprises the step of administering a conjugate described herein to a cell in an amount sufficient to inhibit expression of the target gene. In a preferred embodiment, the present invention further relates to a use of a conjugate described herein for inhibiting expression of a target gene in a target cell in vitro.

Exemplary target genes include, but are not limited to, β-catenin (CTNNB1), IRF4, Factor VII, Eg5, PCSK9, TPX2, apoB, SAA, TTR, RSV, PDGF beta gene, Erb-B gene, Src gene, CRK gene, GRB2 gene, RAS gene, MEKK gene, INK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

In some aspects, a conjugate described herein can be used for treatment of a subject or mammal. For example, the conjugates described herein can be used for treatment of various and other diseases by targeting and killing cells that express a particular tumor antigen. The conjugates can broadly be used for the treatment of any of a variety of cancers. It is anticipated that any type of tumor and any type of tumor-associated antigen can be targeted by the subject conjugates. Examples of cancer types include, without limitation, hematologic cancers, carcinomas, sarcomas, melanoma, and central nervous system cancers.

Non-limiting examples of hematologic cancers that can be treated with the subject immunoconjugates include leukemia, acute myeloid leukemia, acute lymphoblastic leukemia, chronic myelogenous leukemia, chronic lymphocytic leukemia, lymphoma, Hodgkin's lymphoma, non-Hodgkin's lymphoma, multiple myeloma, plasma cell leukemia, and myelodysplasia syndrome.

Non-limiting examples of carcinomas that can be treated with the subject immunoconjugates include skin cancer, head and neck, thyroid, lung, nasopharyngeal, colorectal, liver, urinary bladder, ovarian, cervical, endometrial, prostate, gastric, esophageal, pancreatic, renal, and breast cancer.

Non-limiting examples of sarcomas that can be treated with the subject immunoconjugates include angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, Kaposi's sarcoma and synovial sarcoma.

Non-limiting examples of central nervous system cancers that can be treated with the subject immunoconjugates include glioma, meningioma and neuroma.

Non-limiting examples of other cancers that can be treated with the subject immunoconjugates include melanoma.

In some instances, methods of use of the subject conjugates involve administering a conjugate described herein to a subject in conjunction with one or more additional therapies to treat a particular cancer. As such, a subject conjugate can be used alone to treat a particular cancer, or alternatively, can be used in combination with or as an adjunct to conventional treatment with other medications, e.g., anti-neoplastic agents. Immunoconjugates can generally be used in combination with any anti-neoplastic agents, such as conventional and/or experimental chemotherapeutic agents, radiation treatments, and the like.

For example, in some aspects, an additional therapy can include an antibody, an anti-neoplastic agent, a cytotoxic agent, an anti-angiogenic agent, or an immunosuppressive agent. Non-limiting examples of additional therapeutic agents include cisplatin, carboplatin, oxaliplatin, mechlorethamine, cyclophosphamide, chlorambucil, ifosfamide, doxorubicin, daunorubicin, valrubicin, idarubicin, epirubicin, actinomycin, bleomycin, plicamycin, mitomycin, bevacizumab, imatinib, erlotinib, gefitinib, ibrutinib, idelalisib, lenalidomide, vincristine, vinblastine, vinorelbine, vindesine, paclitaxel, and docetaxel.

### Pharmaceutical Compositions

For therapeutic uses, conjugates described herein can be formulated into pharmaceutical compositions. Accordingly, in another aspect, the invention provides a pharmaceutical composition comprising a conjugate as defined herein. Pharmaceutically acceptable compositions comprise a therapeutically-effective amount of one or more of the conjugates described herein, taken alone or formulated together with one or more pharmaceutically acceptable carriers (additives), excipient and/or diluents.

The pharmaceutical compositions can be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a conjugate described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical carriers include sterile aqueous solutions or dispersions and sterile powders for the extemporaneous preparation of sterile injectable solutions or dispersion. The use of such media and agents for pharmaceutically active substances is known in the art.

The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a conjugate described herein. In certain embodiments, an aforementioned formulation renders orally bioavailable a conjugate described herein.

The conjugate preparation can be formulated in combination with another agent, e.g., another therapeutic agent or an agent that stabilizes the conjugate. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The compounds according to the invention may be formulated for administration in any convenient way for use in human or veterinary medicine, by analogy with other pharmaceuticals.

Compositions can also contain adjuvants such as preservatives, wetting agents, emulsifying agents and/or dispersing agents. Prevention of the presence of microorganisms can be ensured both by sterilization procedures and by the inclusion of various antibacterial and antifungal agents, for example, paraben, chlorobutanol, phenol, sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption, such as aluminum monostearate and gelatin.

A composition must be sterile and fluid to the extent that the composition is deliverable by syringe. In addition to water, the carrier preferably is an isotonic buffered saline solution.

### Routes of Administration

The conjugates described herein or a pharmaceutical composition comprising same can be administered by a variety of methods known in the art. As will be appreciated by the skilled artisan, the route and/or mode of administration will vary depending upon the target disease or condition and the desired results. To administer a conjugate described herein by certain routes of administration, it can be necessary to coat the conjugate with, or co-administer the conjugate with, a material to prevent its inactivation. For example, a conjugate can be administered to a subject in an appropriate carrier, for example, liposomes, or a diluent. Pharmaceutically acceptable diluents include saline and aqueous buffer solutions.

Exemplary routes for administration include, but are not limited to, intravenous, subcutaneous, intratumoral, topical, rectal, anal, vaginal, nasal, pulmonary, and ocular.

The compositions of the present invention can be administered in a number of ways depending upon whether local or systemic treatment is desired and upon the area to be treated. Administration can be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration can be chosen to enhance targeting. For example, to target muscle cells, intramuscular injection into the muscles of interest would be a logical choice. Lung cells might be targeted by administering the conjugate in aerosol form. The vascular endothelial cells could be targeted by coating a balloon catheter with the conjugate and mechanically introducing the conjugate.

### Dosage

Actual dosage levels of the active ingredients, e.g., the conjugate described herein, in the pharmaceutical compositions of the present invention can be varied so as to obtain an amount of the active ingredient which is effective to achieve the desired therapeutic response for a particular patient, composition, and mode of administration, without being toxic to the patient. A selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of dsRNA molecule (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of dsRNA molecule per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The conjugates described herein can be administered to mammals, particularly large mammals such as nonhuman primates or humans in a number of ways.

In some embodiments, the administration of the conjugate is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose.

### Liposomes and Lipid Formulations

The conjugates described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include a conjugate described herein are delivered into the cell where the dsRNA can specifically bind to a target RNA and can mediate RNAi. In some cases, the liposomes are also specifically targeted, e.g., to direct the conjugate to particular cell types.

A liposome containing a conjugate described herein can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The siRNA preparation is then added to the micelles that include the lipid component. The cationic groups on the lipid interact with the dsRNA of the conjugate and condense around the conjugate to form a liposome. After condensation, the detergent is removed, e.g., by dialysis, to yield a liposomal preparation of siRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2, 405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with siRNA are useful for treating a dermatological disorder.

Liposomes that include a conjugate described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include siRNA can be delivered, for example, subcutaneously by infection in order to deliver siRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. Surfactants find wide application in formulations such as emulsions (including microemulsions) and liposomes (see above). A conjugate formulation can include a surfactant. In some embodiments, a conjugate described herein is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, N.Y., 1988, p. 285).

Micelles and other Membranous Formulations. Formulations comprising a conjugate described herein can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains conjugate described herein and at least the alkali metal alkyl sulphate. The first micellar composition is then mixed with at least three micelle forming compounds to form a mixed micellar composition. In another method, the micellar composition is prepared by mixing conjugate described herein, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, conjugate described herein can be incorporated into a particle, e.g., a microparticle. Microparticles can be produced by spray-drying, but may also be produced by other methods including lyophilization, evaporation, fluid bed drying, vacuum drying, or a combination of these techniques.

### Kits

The invention also provides kits comprising the conjugates described herein.

In some embodiments, the kit further comprises instructions for use.

Exemplary embodiments of the various aspects described herein can be illustrated by the following numbered embodiments:

Embodiment 1: A conjugate comprising: (a) a dual variable domain immunoglobulin molecule (Ig), or an antigen-binding fragment thereof, wherein the dual variable domain immunoglobulin molecule comprises: (i) a first variable domain that binds to a binding target, and (ii) a second variable domain that comprises a reactive residue; (b) a linker covalently conjugated to the reactive residue of the second variable domain of the Ig; and (c) a double-stranded RNA (dsRNA) molecule conjugated to the linker. Optionally, the dsRNA is capable of inhibiting the expression of a target gene. Optionally, the dsRNA comprises a sense strand and an antisense strand, each strand having 14 to 40 nucleotides, wherein the antisense strand has sufficient complementarity to the target sequence to mediate RNA interference, Optionally, the dsRNA further has at least one of the following characteristics: (i) a melting temperature (Tm) of from about 40° C. to about 80° C.; (ii) the antisense strand comprises 2, 3, 4, 5 or 6 2′-fluoro modifications; (iii) the antisense strand comprises 1, 2, 3 or 4 phosphorothioate internucleotide linkages; (iv) the sense strand is conjugated with the linker; (v) the sense strand comprises 2, 3, 4 or 5 2′-fluoro modifications; (vi) the sense strand comprises 1, 2, 3 or 4 phosphorothioate internucleotide linkages; (vii) the dsRNA comprises at least four 2′-fluoro modifications; (viii) the dsRNA comprises a duplex region of 12-40 nucleotide pairs in length; (ix) the dsRNA has a blunt end at 5′end of the antisense strand; and (x) the dsRNA has an overhang at 3′-end of the antisense strand.

Embodiment 2: The conjugate according to Embodiment 1, wherein the dsRNA has a melting temperature (Tₘ) of from about 40° C. to about 80° C.

Embodiment 3: The conjugate according to any one of Embodiments 1-2, wherein the dsRNA has a melting temperature of at least 60° C.

Embodiment 4: The conjugate according to any one of Embodiments 1-3, wherein the dsRNA comprises at least four 2′-fluoro modifications.

Embodiment 5: The conjugate according to any one of Embodiments 1-4, wherein the dsRNA comprises a duplex region of 12-40 nucleotide base pairs in length.

Embodiment 6: The conjugate according to any one of Embodiments 1-5, wherein the dsRNA comprises a duplex region of 18-25 nucleotide base pairs in length.

Embodiment 7: The conjugate according to any one of Embodiments 1-6, wherein the dsRNA comprises a blunt end at 5′-end of the antisense strand.

Embodiment 8: The conjugate according to any one of Embodiments 1-7, wherein the dsRNA comprises an overhang at 3′-end of the antisense strand.

Embodiment 9: The conjugate according to any one of Embodiments 1-8, wherein the dsRNA comprises an overhang of at least two nucleotides at 3′-end of the antisense strand.

Embodiment 10: The conjugate according to any one of Embodiments 1-9, wherein the sense strand is covalently conjugated with the linker.

Embodiment 11: The conjugate according to Embodiment 10, wherein 5′-end of the sense stand is covalently conjugated with the linker.

Embodiment 12: The conjugate of Embodiment 10, wherein 3′-end of the sense stand is covalently conjugated with the linker.

Embodiment 13: The conjugate according to any one of Embodiments 1-12, wherein the sense strand is 19-25 nucleotides in length.

Embodiment 14: The conjugate according to any one of Embodiments 1-13, wherein the sense strand is 21 nucleotides in length.

Embodiment 15: The conjugate according to any one of Embodiments 1-14, wherein the sense strand comprises 2, 3, 4 or 5 2′-fluoro modifications.

Embodiment 16: The conjugate according to any one of Embodiments 1-15, wherein the sense strand comprises 3 or 4 2′-fluoro modifications.

Embodiment 17: The conjugate according to any one of Embodiments 1-16, wherein the sense strand comprises 2′-fluoro modifications at positions 7, 10 and 11, counting from the 5′-end.

Embodiment 18: The conjugate according to any one of Embodiments 1-17, wherein the sense strand comprises 2′-fluoro modifications at positions 7, 9, 10 and 11, counting from the 5-end.

Embodiment 19: The conjugate according to any one of Embodiments 1-18, wherein the sense strand comprises 0, 1, 2, 3 or 4 phosphorothioate internucleotide linkages.

Embodiment 20: The conjugate according to any one of Embodiments 1-19, wherein the sense stand comprises phosphorothioate internucleotide linkages between nucleotide positions 1 and 2, and between nucleotide positions 2 and 3, counting from the 5′ end.

Embodiment 21: The conjugate according to any one of Embodiments 1-20, wherein the antisense strand is 19-25 nucleotides in length.

Embodiment 22: The conjugate according to any one of Embodiments 1-21, wherein the antisense is 23 nucleotides in length.

Embodiment 23: The conjugate according to any one of Embodiments 1-22, wherein the Embodiment 3: antisense comprises 2, 3, 4, 5 or 6 2′-fluoro modifications.

Embodiment 24: The conjugate according to any one of Embodiments 1-23, wherein the antisense comprises 2′-fluoro modifications at positions 2, 14 and 16, counting from the 5′-end.

Embodiment 25: The conjugate according to any one of Embodiments 1-24, wherein the antisense comprises 2′ fluoro modifications at positions 2, 6, 9, 14 and 16, counting from the 5′-end.

Embodiment 26: The conjugate according to any one of Embodiments 1-25, wherein the antisense comprises 2′-fluoro modifications at positions 2, 6, 8, 9, 14 and 16, counting from the 5′-end.

Embodiment 27: The conjugate according to any one of Embodiments 1-26, wherein the antisense comprises 1, 2, 3 or 4 phosphorothioate internucleotide linkages.

Embodiment 28: The conjugate according to any one of Embodiments 1-27, wherein the antisense comprises phosphorothioate internucleotide linkages between nucleotide positions 21 and 22, and between nucleotide positions 22 and 23, counting from the 5′ end.

Embodiment 29: The conjugate according to any one of Embodiments 1-28, wherein the antisense comprises phosphorothioate internucleotide linkages between nucleotide positions 1 and 2, between nucleotide positions 2 and 3, between nucleotide positions 21 and 22, and between nucleotide positions 22 and 23, counting from the 5′ end.

Embodiment 30: The conjugate according to any one of Embodiments 1-29, wherein the antisense strand comprises at least one thermally destabilizing modification of the duplex within the first 9 nucleotide positions of the 5′ region.

Embodiment 31: The conjugate of Embodiment 30, wherein said thermally destabilizing modification is at position 4, 5, 6, 7, 8 or 9, counting from 5′-end, of the antisense strand.

Embodiment 32: The conjugate of Embodiment 31, wherein said thermally destabilizing modification is at position 7, counting from 5′-end, of the antisense strand.

Embodiment 32: The conjugate according to any one of Embodiments 1-32, wherein the antisense comprises a 5′-vinylphosphonate nucleotide at 5′-end.

Embodiment 34: The conjugate according to any one of Embodiments 1-33, wherein the dsRNA comprises at least one 2′-OMe modification.

Embodiment 35: The conjugate according to any one of Embodiments 1-34, wherein the sense strand comprises at least one 2′-OMe modification.

Embodiment 36: The conjugate according to any one of Embodiments 1-35, wherein the antisense strand comprises at least one 2′-OMe modification.

Embodiment 37: The conjugate according to any one of Embodiments 1-36, wherein the dsRNA comprises at least one locked nucleic acid (LNA) modification.

Embodiment 38: The conjugate according to any one of Embodiments 1-37, wherein the reactive residue is a lysine.

Embodiment 39: The conjugate according to any one of Embodiments 1-38, wherein the first variable domain of Ig is positioned closer to an N-terminus than the second variable domain.

Embodiment 40: The conjugate according to any one of Embodiments 1-39, wherein Ig is a bispecific immunoglobulin molecule.

Embodiment 41: The conjugate according to any one of Embodiments 1-40, wherein the antigen-binding fragment comprises the first and second variable domains of Ig, and is selected from a Fab, Fab′, F(ab′)₂, Fv or scFv.

Embodiment 42: The conjugate according to any one of Embodiments 1-41, wherein the antigen-binding fragment comprises a Fab.

Embodiment 43: The conjugate according to any one of Embodiments 1-42, wherein Ig comprises a chimeric immunoglobulin sequence.

Embodiment 44: The conjugate according to any one of Embodiments 1-43, wherein Ig comprises a humanized immunoglobulin sequence,

Embodiment 45: The conjugate according to any one of Embodiments 1-44, wherein Ig comprises a human immunoglobulin sequence.

Embodiment 46: The conjugate according to any one of Embodiments 1-45, wherein the binding target is a tumor cell surface antigen.

Embodiment 47: The conjugate of any one of Embodiments 1-46, wherein the first variable domain binds to CD138, B-cell maturation antigen (BCMA), SLAMF7, HER2, FOLR1, or CD79b.

Embodiment 48: The conjugate according to any one of Embodiments 1-47, wherein L is a reversible linker.

Embodiment 49: The conjugate according to any one of Embodiments 1-48, wherein L is an irreversible linker.

Embodiment 50: The conjugate according to any one of Embodiments 1-49, wherein L is a cleavable linker.

Embodiment 51: The conjugate according to any one of Embodiments 1-50, wherein L is a non-cleavable linker.

Embodiment 52: The conjugate according to any one of Embodiments 1-51, wherein L is a branched linker.

Embodiment 53: The conjugate according to any one of Embodiments 1-52, wherein L is a linear linker.

Embodiment 54: The conjugate according to any one Embodiments, 1-53, wherein the Ig comprises a first heavy chain and light chain.

Embodiment 55: The conjugate according to any one Embodiments, 1-54, wherein the Ig comprises a first heavy chain, a second heavy chain and a light chain, wherein the first heavy chain and the second heavy chain are different.

Embodiment 56: The conjugate according to any one Embodiments, 1-55, wherein the Ig is capable of binding two different epitopes.

Embodiment 57: The conjugate according to any one Embodiments, 1-54, wherein the Ig comprises a heavy chain, a light chain, and a J chain.

Embodiment 58: The conjugate according to any one of Embodiments 1-57, wherein the Ig further comprises a ligand.

Embodiment 59: The conjugate according to Embodiment 58, wherein the ligand is an endosomolytic ligand.

Embodiment 60: The conjugate according to Embodiment 59, wherein the ligand is linked to the light chain.

Embodiment 61: The conjugate according to any one of Embodiments 1-60, wherein one of the variable domain, e.g., first or second variable domain comprises an amino acid sequence selected from the group consisting of SEQ ID NO: 9, SEQ ID NO: 10, SEQ ID NO: 11, SEQ ID NO: 12, SEQ ID NO: 13, SEQ ID NO: 14, and any combinations thereof.

Embodiment 62: The conjugate of any one of Embodiments 1-61, wherein the Ig comprises a peptide linker between two of the domains, e.g., the first variable domain and the second variable domain.

Embodiment 63: The conjugate of Embodiment 62, wherein the peptide linker comprises an amino acid sequence selected from the group consisting of ASTKGP (SEQ IS NO: 1), TVAAPSVFIFPP (SEQ IS NO: 2), GIS (SEQ IS NO: 3), (G₄S)₂ (SEQ IS NO: 4), (G₄S)₃ (SEQ IS NO: 5), EPKSCDG₄S (SEQ IS NO: 6), EPKSCD(G₄S)₂ (SEQ IS NO: 7), EPKSCD(G₄S)₃ (SEQ IS NO: 8), and any combinations thereof.

Embodiment 64: The conjugate of any one of Embodiments 1-63, wherein the Ig comprises an amino acid sequence selected from the group consisting of SEQ ID NO: 15, SEQ ID NO: 16, SEQ ID NO: IT, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and any combinations thereof.

Embodiment 65: A pharmaceutical composition comprising the conjugate of according to any one of Embodiments 1-64 alone or in combination with a pharmaceutically acceptable carrier or excipient.

Embodiment 66: A gene silencing kit comprising the conjugate according to any one of claims 1-64.

Embodiment 67: A method for silencing a target gene in a cell, the method comprising introducing a conjugate according to any one of Embodiments 1-64 into the cell.

Embodiment 68: Use of the conjugate according to any one of Embodiments 1-64 in the preparation of a medicament.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences typically differ by at least 5 nucleotides.

In some embodiments, a dsRNA molecule is “sufficiently complementary” to a target RNA, e.g., a target mRNA, such that the dsRNA molecule silences production of protein encoded by the target mRNA. In another embodiment, the dsRNA molecule is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA molecule specifically discriminates a single-nucleotide difference. In this case, the dsRNA molecule only mediates RNAi if exact complementary is found in the region (e.g., within 7 nucleotides of) the single-nucleotide difference.

As used herein, the term “oligonucleotide” refers to a nucleic acid molecule (RNA or DNA) for example of length less than 100, 200, 300, or 400 nucleotides.

The term ‘BNA’ refers to bridged nucleic acid, and is often referred as constrained or inaccessible RNA. BNA can contain a 5-, 6-membered, or even a 7-membered bridged structure with a “fixed” C3′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

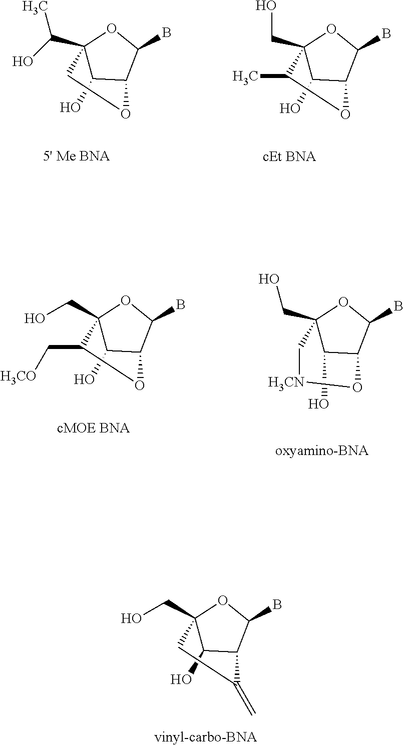

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

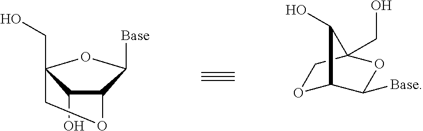

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “immunoglobulin” or “antibody” as used interchangeably herein refers to a basic 4-chain heterotetrameric glycoprotein composed of two identical light (L) chains and two identical heavy (H) chains. Each L chain is linked to an H chain by one covalent disulfide bond, while the two H chains are linked to each other by one or more disulfide bonds depending on the H chain isotype. Each H and L chain has an N-terminus and a C-terminus, and also has regularly spaced intrachain disulfide bridges. Each H chain has at the N-terminus a variable domain (V_{H}) followed by three constant domains (C_{H}1, C_{H}2 and C_{H}3). Each L chain has at the N-terminus a variable domain (V_{L}) followed by one constant domain (C_{L}). The V_{L} is aligned with the V_{H} and the C_{L} is aligned with the first constant domain of the heavy chain (C_{H}1). Particular amino acid residues are believed to form an interface between the L chain and H chain variable domains. The pairing of a V_{H} and V_{L} together forms a single antigen-binding site.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ϵ, γ, and μ, respectively. The γ and a classes are further divided into subclasses on the basis of relatively minor differences in CH sequence and function, e.g., humans express the following subclasses: IgG1, IgG2, IgG3, IgG4, IgA1, and IgA2.

The “variable region” or “variable domain” of an immunoglobulin refers to the N-terminal domains of the H or L chain of the immunoglobulin. The variable domain of the H chain can be referred to as “V_{H}” The variable domain of the light chain can be referred to as “V_{L}.” These domains are generally the most variable parts of an immunoglobulin and contain the antigen-binding sites.

The term “variable” refers to the fact that certain segments of the variable domains differ extensively in sequence among immunoglobulins. The V domain mediates antigen binding and defines specificity of a particular immunoglobulin for its particular antigen. However, the variability is not evenly distributed across the 110-amino acid span of most variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native H and L chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of immunoglobulins (see Kabat et al., Sequences of Proteins of Immunological interest, 5th Ed. Public Health Service, National Institutes of Health, Bethesda, Md. (1991)). The constant domains are not involved directly in binding an immunoglobulin to an antigen, but exhibit various effector functions, such as participation of the immunoglobulin in antibody dependent cellular cytotoxicity (ADCC), antibody-dependent cellular phagocytosis (ADCP), and complement-dependent cytotoxicity (CDC).

An “intact” immunoglobulin is one that comprises an antigen-binding site as well as a C_{L} and at least H chain constant domains, C_{H} I, C_{H} and C_{H}3. The constant domains can be native sequence constant domains (e.g., human native sequence constant domains) or amino acid sequence variants thereof. An intact immunoglobulin can have one or more effector functions.

A “naked immunoglobulin” for the purposes herein is an immunoglobulin that is not conjugated to a dsRNA molecule.

“Immunoglobulin fragments” comprise a portion of an intact immunoglobulin, preferably the antigen binding or variable region of the intact immunoglobulin. Examples of immunoglobulin fragments include, but are not limited to, Fab, Fab′, F(ab′)₂, and Fv fragments; diabodies; linear immunoglobulins (see U.S. Pat. No. 5,641,870, Example 2; Zapata et al., Protein Eng. 8(10): 1057-1062 [1995]); single-chain immunoglobulin molecules; and multispecific immunoglobulins formed from immunoglobulin fragments. In some aspects, the immunoglobulin fragments include all possible alternate fragment formats. In some aspects, the immunoglobulin fragments may be bispecific. In some aspects, the immunoglobulin fragments may be bi-paratopic. In some aspects, the immunoglobulin fragments may be trispecific. In some aspects, the immunoglobulin fragments may be multimeric. In some aspects, an immunoglobulin fragment comprises an antigen binding site of the intact immunoglobulin and thus retains the ability to bind antigen. In some aspects, the immunoglobulin fragment contains single variable domains which have the ability to bind antigen. In some aspects, the immunoglobulin fragments are further modified (not limited to peptide addition, pegylation, hesylation, glycosylation) to modulate activity, properties, pharmacokinetic behavior and in vivo efficacy.

Papain digestion of immunoglobulins produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}I). Each Fab fragment is monovalent with respect to antigen binding, i.e., it has a single antigen-binding site. Pepsin treatment of an immunoglobulin yields a single large F(ab′)₂ fragment which roughly corresponds to two disulfide linked Fab fragments having divalent antigen-binding activity and is still capable of cross-linking antigen Fab′ fragments differ from Fab fragments by having additional few residues at the carboxy terminus of the C_{H}1 domain including one or more cysteines from the immunoglobulin hinge region. Fab′-SH is the designation herein for Fab′ in which the cysteine residue(s) of the constant domains bear a free thiol group. F(ab′)₂ immunoglobulin fragments originally were produced as pairs of Fab′ fragments which have hinge cysteines between them. Other chemical couplings of immunoglobulin fragments are also known.

The Fc fragment comprises the carboxy-terminal portions of both H chains held together by disulfides. The effector functions of immunoglobulins are determined by sequences in the Fc region, which region is also the part recognized by Fe receptors (FcR) found on certain types of cells.

“Fv” is the minimum immunoglobulin fragment which contains a complete antigen-recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the immunoglobulin. However, even a single variable domain (or half of an Fv comprising only three CDRs specific for an antigen) has the ability to recognize and bind antigen, although typically at a lower affinity than the entire binding site. When used herein in reference to a DVD immunoglobulin molecule, the term “Fv” refers to a binding fragment that includes both the first and the second variable domains of the heavy chain and the light chain.

“Single-chain Fv” also abbreviated as “sFv” or “scFv” are immunoglobulin fragments that comprise the V_{H} and V_{L} immunoglobulin domains connected into a single polypeptide chain. Preferably, the sFv polypeptide further comprises a polypeptide linker between the V_{H} and V_{L} domains which enables the sFv to form the desired structure for antigen binding. For a review of sFv, see Plückthun in The Pharmacology of Monoclonal Antibodies, vol. 113, Rosenburg and Moore eds., Springer-Verlag, New York, pp. 269-315 (1994); Borrebaeck 1995, infra. When used herein in reference to a DVD immunoglobulin molecule, the term “scFv” refers to a binding fragment that includes both the first and the second variable domains of the heavy chain and the light chain.

The term “dual variable domain immunoglobulin” or “DVD-Ig” as used herein refers to an immunoglobulin molecule as described above, wherein both the H and L chains include a second variable domain located adjacent to the first variable domain. The L chain of a DVD-Ig therefore includes, from N-terminus to C-terminus, the following domains: V_{L}1-V_{L}2-C_{L}. The H chain of a DVD-Ig therefore includes, from N-terminus to C-terminus, the following domains: V_{H}1-V_{H}2-C_{H}1-C_{H}2-C_{H}3. The pairing of a V_{L}I and V_{H} I together forms a first antigen-binding site. The pairing of a V_{L}2 and V_{H}2 together forms a second antigen binding site.

Unless stated otherwise, the term “immunoglobulin” or “antibody” specifically includes native human and non-human IgG1, IgG2, IgG3, IgG4, IgE, IgA1, IgA2, IgD and IgM antibodies, including naturally occurring variants.

The term “native” with reference to a polypeptide (e.g., an antibody or immunoglobulin) is used herein to refer to a polypeptide having a sequence that occurs in nature, regardless of its mode of preparation. The term “non-native” with reference to a polypeptide (e.g., an antibody or immunoglobulin) is used herein to refer to a polypeptide having a sequence that does not occur in nature.

The term “polypeptide” is used herein in the broadest sense and includes peptide sequences. The term “peptide” generally describes linear molecular chains of amino acids containing up to about 30, preferably up to about 60 amino acids covalently linked by peptide bonds.

The term “monoclonal” as used herein refers to an antibody or immunoglobulin molecule (e.g., a DVD Ig molecule) obtained from a population of substantially homogeneous immunoglobulins, i.e., the individual immunoglobulins comprising the population are identical except for possible naturally occurring mutations that can be present in minor amounts. Monoclonal immunoglobulins are highly specific, being directed against a single antigenic site. Furthermore, in contrast to conventional (polyclonal) antibody preparations which typically include different antibodies directed against different determinants (epitopes), each monoclonal immunoglobulin is directed against a single determinant on the antigen. The modifier “monoclonal” indicates the character of the immunoglobulin as being obtained from a substantially homogeneous population of immunoglobulins, and is not to be construed as requiring production of the antibody by any particular method. For example, the monoclonal immunoglobulins in accordance with the present invention can be made by the hybridoma method first described by Kohler and Milstein (1975) Nature 256:495, or can be made by recombinant DNA methods (see, e.g., U.S. Pat. No. 4,816,567).

The monoclonal immunoglobulins herein specifically include “chimeric” immunoglobulins in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity (U.S. Pat. No. 4,816,567; and Morrison et al. (1984) Proc. Natl. Acad. Sci. USA 81:6851-6855).

“Humanized” forms of non-human (e.g., rodent, e.g., murine or rabbit) immunoglobulins are immunoglobulins which contain minimal sequences derived from non-human immunoglobulin. For the most part, humanized immunoglobulins are human immunoglobulins (recipient antibody) in which residues from a hypervariable region of the recipient are replaced by residues from a hypervariable region of a non-human species (donor antibody) such as mouse, rat, hamster, rabbit, chicken, bovine or non-human primate having the desired specificity, affinity, and capacity. In some instances, Fv framework region (FR) residues of the human immunoglobulin are also replaced by corresponding non-human residues. Furthermore, humanized antibodies can comprise residues which are not found in the recipient antibody or in the donor antibody. These modifications are made to further refine antibody performance. In general, the humanized immunoglobulin will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized immunoglobulin optionally also will comprise at least a portion of an immunoglobulin constant region (Fe), typically that of a human immunoglobulin. For further details, see Jones et al. (1986) Nature 321:522-525; Riechnmann et al. (1988) Nature 332:323-329; and Presta (1992) Curr. Op. Struct. Biol. 2:593-596.

The term “human immunoglobulin”, as used herein, is intended to include immunoglobulins having variable and constant regions derived from human germline immunoglobulin sequences. The human immunoglobulins of the invention can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human immunoglobulin”, as used herein, is not intended to include immunoglobulins in which CDR sequences derived from the germline of another mammalian species, such as a mouse, have been grafted onto human framework sequences.

An “isolated” immunoglobulin herein is one which has been identified and separated and/or recovered from a component of its natural environment in a recombinant host cell. Contaminant components of its natural environment are materials which would interfere with diagnostic or therapeutic uses for the immunoglobulin, and can include enzymes, hormones, and other proteinaceous or nonproteinaceous solutes, as well as undesired byproducts of the production. In some aspects, an isolated immunoglobulin herein will be purified (1) to greater than 95% by weight, or greater than 98% by weight, or greater than 99% by weight, as determined by SDS-PAGE or SEC-HPLC methods, (2) to a degree sufficient to obtain at least 15 residues of N-terminal or internal amino acid sequence by use of an amino acid sequencer, or (3) to homogeneity by SDS-PAGE under reducing or non-reducing conditions using Coomassie blue or, preferably, silver stain. Ordinarily, an isolated immunoglobulin will be prepared by at least one purification step.

The term “specific binding” or “specifically binds to” or is “specific for” refers to the binding of a binding moiety to a binding target, such as the binding of an immunoglobulin to a target antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety, or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a molecule binds to a particular polypeptide or epitope on a particular polypeptide without substantially binding to any other polypeptide or polypeptide epitope.

“Binding affinity” refers to the strength of the sum total of noncovalent interactions between a single binding site of a molecule (e.g., an immunoglobulin) and its binding partner (e.g., an antigen). Unless indicated otherwise, as used herein, “binding affinity” refers to intrinsic binding affinity which reflects a 1:1 interaction between members of a binding pair (e.g., immunoglobulin and antigen). The affinity of a molecule X for its partner Y can generally be represented by the dissociation constant (K_{d}). For example, the K_{d} can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger. Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art.

As used herein, the “K_{d}” or “K_{d} value” refers to a dissociation constant measured by a technique appropriate for the immunoglobulin and target pair, for example using surface plasmon resonance assays, for example, using a Biacore X100 or a Biacore T200 (GE Healthcare, Piscataway, N.J.) at 25° C. with immobilized antigen CM5 chips.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini. The term “fusion” is used herein to refer to the combination of amino acid sequences of different origin

The term “epitope” includes any molecular determinant capable of specific binding to an immunoglobulin. In certain aspects, epitope determinants include chemically active surface groupings of molecules such as amino acids, sugar side chains, phosphoryl, or sulfonyl, and, in certain aspects, can have specific three dimensional structural characteristics, and/or specific charge characteristics. An epitope is a region of an antigen that is bound by an immunoglobulin. A “binding region” is a region on a binding target bound by a binding molecule.

The term “target” or “binding target” is used in the broadest sense and specifically includes polypeptides, without limitation, nucleic acids, carbohydrates, lipids, cells, and other molecules with or without biological function as they exist in nature.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

An “antigen-binding site” or “antigen-binding region” of an immunoglobulin of the present invention typically contains six complementarity determining regions (CDRs) within each variable domain, and which contribute in varying degrees to the affinity of the binding site for antigen. In each variable domain there are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences and/or structural information from antibody/antigen complexes. Also included within the scope of the invention are functional antigen binding sites comprised of fewer CDRs (i.e., where binding specificity is determined by three, four or five CDRs). Less than a complete set of 6 CDRs can be sufficient for binding to some binding targets. Thus, in some instances, the CDRs of a V_{H} or a V_{L} domain alone will be sufficient. Furthermore, certain antibodies might have non-CDR-associated binding sites for an antigen. Such binding sites are specifically included within the present definition.

The term “host cell” as used in the current application denotes any kind of cellular system which can be engineered to generate the immunoglobulins according to the current invention. In one aspect, Chinese hamster ovary (CHO) cells are used as host cells. In some aspects, E. coli are used as host cells.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations, Variant progeny that have the same function or biological activity as screened for in the originally transformed cell are included.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a peptide or polypeptide sequence, i.e., the h38C2 antibody polypeptide sequences identified herein, is defined as the percentage of amino acid residues in a candidate sequence that are identical with the amino acid residues in the specific peptide or polypeptide sequence after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

“Treating” or “treatment” refers to both therapeutic treatment and prophylactic or preventative measures, wherein the object is to prevent or slow down (lessen) a targeted pathologic condition or disorder. For example, a subject or mammal is successfully “treated” for cancer, if, after receiving a therapeutic amount of a conjugate described herein, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The invention is further illustrated by the following examples, which should not be construed as further limiting. The contents of all references, pending patent applications and published patents, cited throughout this application are hereby expressly incorporated by reference.

### Examples

### Example 1: In Vitro Screening of Antibody-siRNA Conjugates

RNA interference (RNAi) enables the selective knockdown of any disease-related RNA-based factor making it a powerful strategy for the treatment of cancer. However, there are several challenges that limit RNAi as a therapeutic. Herein, we report a method to generate antibody-siRNA conjugates (ARCs) that circumvent some of these limitations. This strategy utilizes engineered dual-variable-domain antibodies (DVDs) containing a natural, highly reactive buried lysine residue to generate ARCs that are mutation free and site-specific. Three exemplary DVDs were prepared against SLAMF7, BCMA, and CD138 for the targeting of multiple myeloma (MM) and conjugated with siRNA targeting β-catenin. The BCMA targeting ARC resulted in high β-catenin RNA and protein knockdown, thus validating the ARCs as an RNAi based approach for the treatment of cancer.

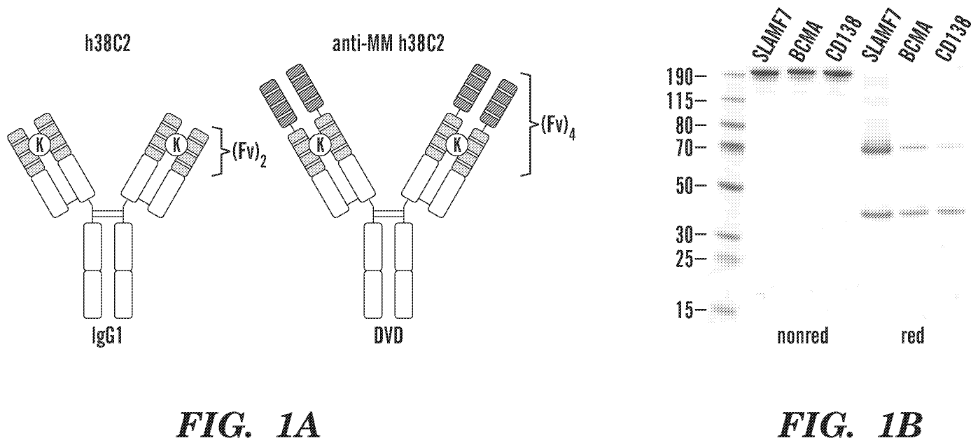

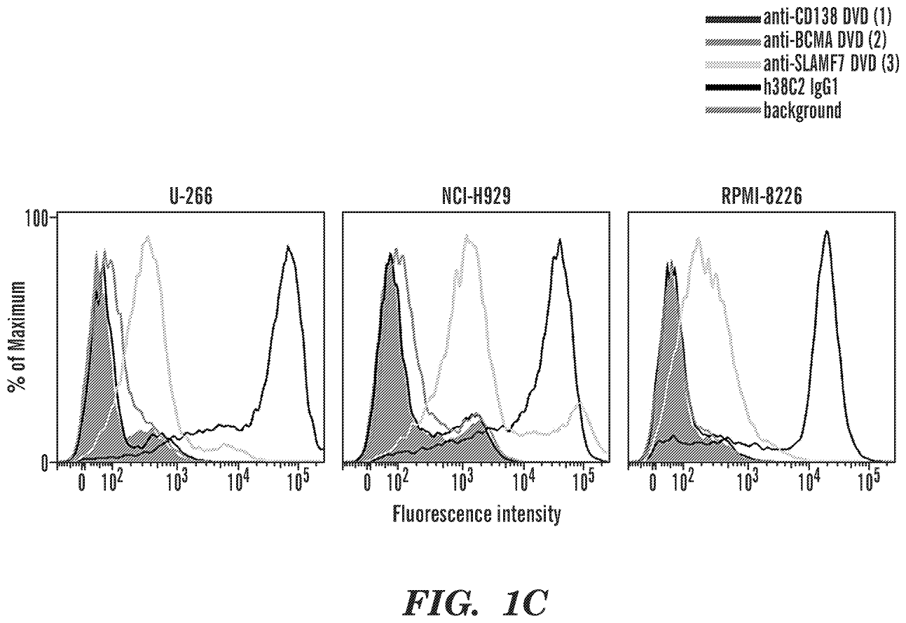

The inventors have developed a site-specific antibody-drug conjugate (ADC) platform by engineering dual-variable-domain (DVD) antibodies composed of an outer variable light (VL) and heavy chain (VH) domain pair (Fv) that selectively targets a cell surface antigen of interest and an inner Fv derived from the anti-hapten mAb h38C2 which contains a uniquely reactive lysine (Lys) residue at the bottom of an 11-Å deep hydrophobic pocket. Due to its distinctive environment, this Lys is more nucleophilic (pKa ˜6) and reacts specifically with β-lactam functionalized hapten derivatives. When made as IgG1, there are two binding sites (outer Fv) and two drug attachment sites (inner Fv) within one DVD molecule (FIG. 1A). The inventor used the DVD platform to generate highly homogeneous antibody-drug conjugates (ADCs) that potently and selectively killed tumor cells in vitro and in vivo. Compared to other ADC formats, DVD-ADCs have several advantages including low immunogenicity and ease of conjugation in physiological conditions.¹⁸ In the present work, the inventors selected multiple myeloma (MM), a hematologic malignancy characterized by aberrant growth of plasma cells in the bone marrow.¹⁹ This indication was a suitable place to start as there are several established cell surface antigens expressed in MM that have been successfully targeted by ADCs in clinical trials.⁷

Preparation of MM-targeting antibody-RNA conjugates (ARCs) delivering p3-catenin siRNA: Three DVD-IgG1s (FIG. 1A) targeting human (1) CD138²⁰, (2) B-cell maturation antigen (BCMA),²¹-2³ and (3) SLAMF7,²⁴ were cloned, expressed, and purified. All of these targets are validated MM targets for antibody-based therapeutics and have been clinically investigated with ADCs.⁷ The DVD IgG1s 1-3 were prepared in high purity (FIG. 1B) with retention of specific binding against three MM cell lines (FIG. 1C).

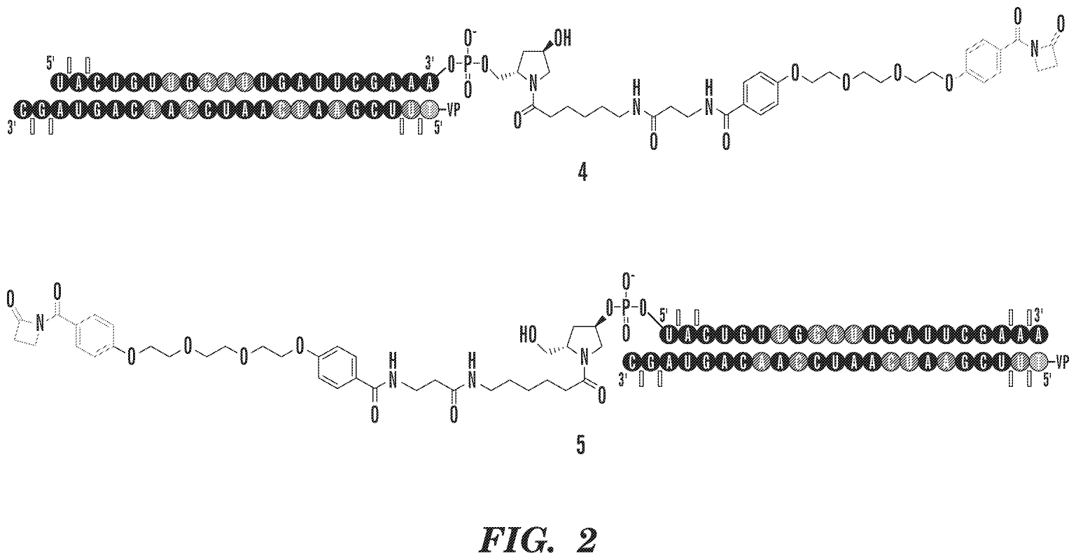

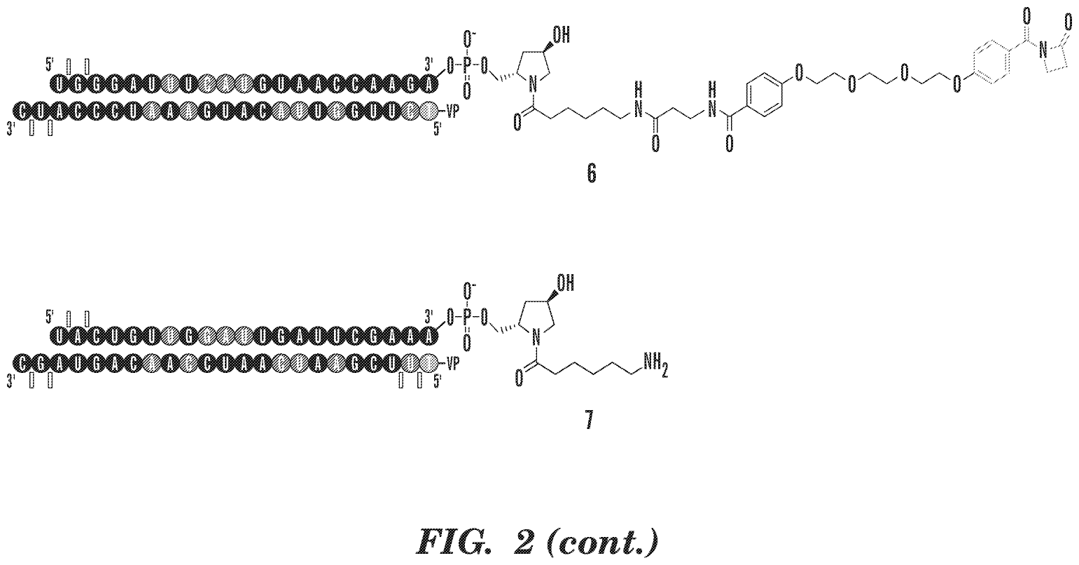

Next, β-lactam hapten functionalized siRNAs (4 and 5) targeting human β-catenin (CTNNB1) mRNA were synthesized. CTNNB1 was chosen as the target gene because it is overexpressed in MM.^{25,26} The β-lactam hapten functionality serves as the reactive handle to conjugate the siRNA to the uniquely reactive Lys residue contained in the inner Fv of the DVD-IgG1s. Two compounds were prepared targeting CTNNB1: an siRNA with the β-lactam hapten handle at the 3′ end (FIG. 2, compound 4) and an siRNA with the β-lactam hapten handle at the 5′ end (FIG. 2, compound 5) of the passenger (sense) strand. The passenger strand was modified with the β-lactam hapten handle in all cases so RISC complex formation would not be effected. In addition to these compounds, two control siRNAs were synthesized as negative controls. One compound is an siRNA targeting human transthyretin (TTR), an irrelevant gene in this study, with a β-lactam hapten handle at the 3′ end (FIG. 2, compound 6) and the other compound is an mRNA targeting CTNNB1 that lacks the β-lactam hapten moiety (FIG. 2, compound 7).^{Error! Bookmark not defined.}

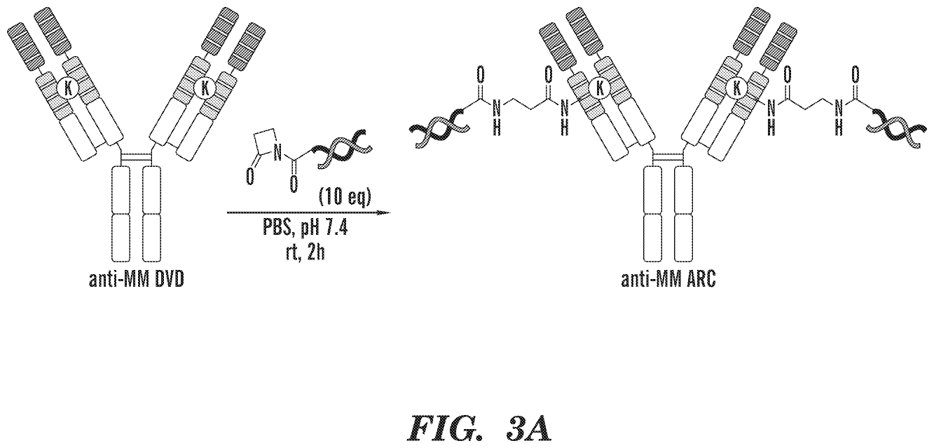

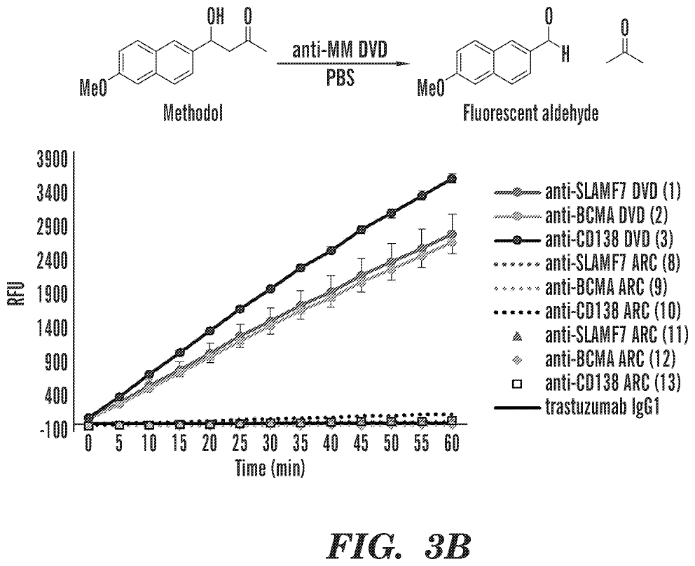

To prepare the desired ARCs each DVD-IgG1 (1-3) was incubated with 10 equivalents of β-lactam-hapten CTNNB1 siRNA (FIG. 2, compounds 4 and 5) in PBS for 2 h (FIG. 3A). The h38C2 Lys reactivity in the DVD-IgG1s was shown to be preserved using an assay directly assessing its catalytic activity through the conversion of methodol to its parent fluorescent aldehyde (FIG. 3B).²⁷ Furthermore, after incubation with the β-lactam-hapten siRNA, the catalytic activity was lost indicating complete siRNA conjugation to the Lys residue (FIG. 3B) to form ARCs 8-13. A total of six ARCs were assembled targeting CTNNB1: ARC 8=1+4, ARC 9=2+4, ARC 10=3+4, ARC 11=1+5, ARC 12=2+5, and ARC 13=3+5.

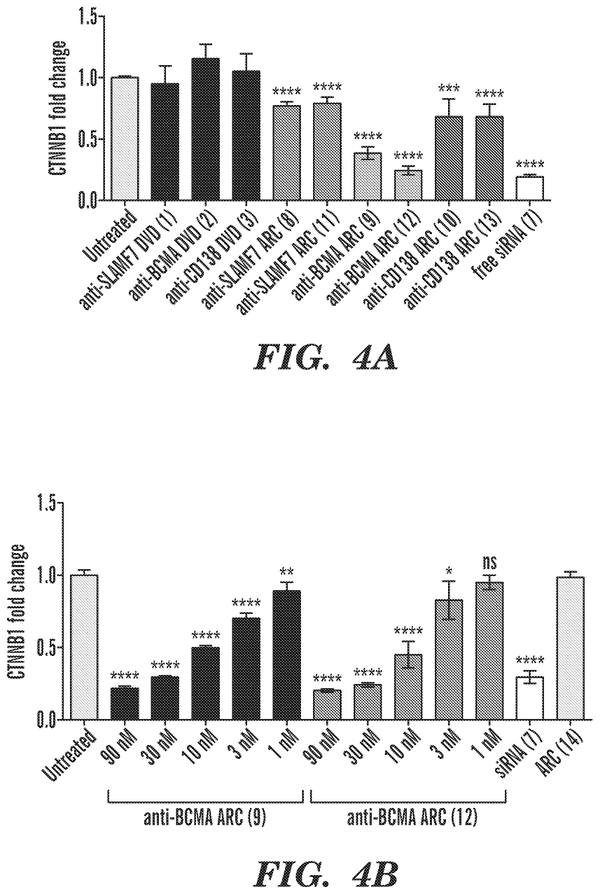

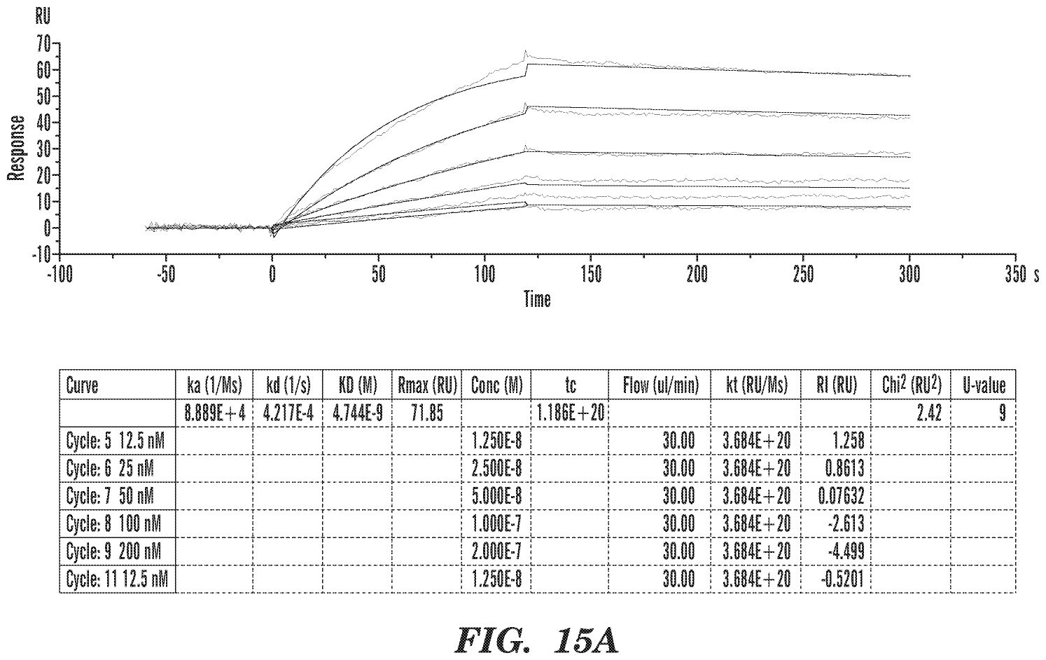

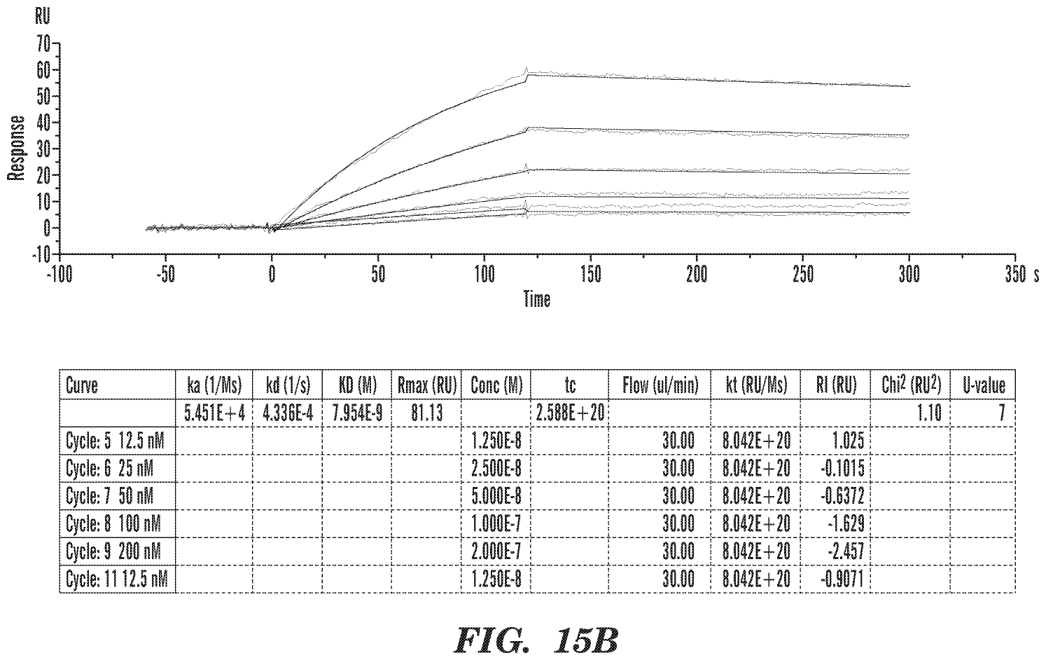

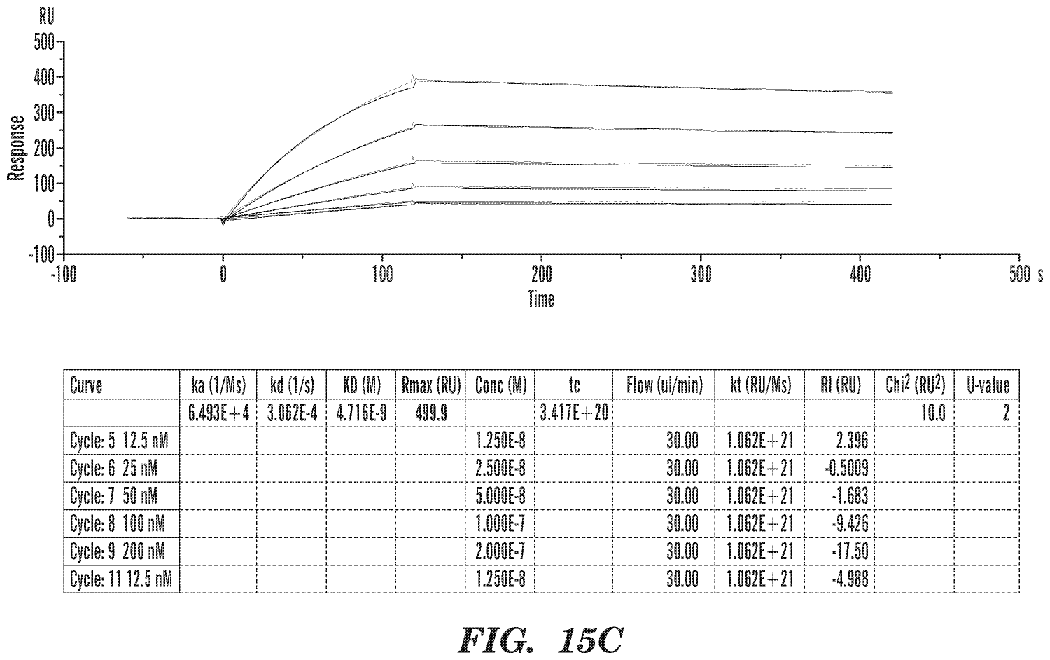

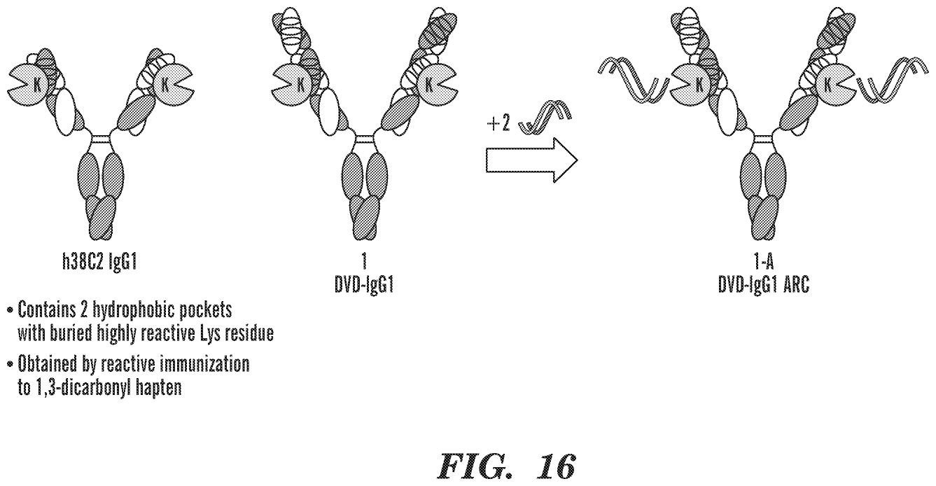

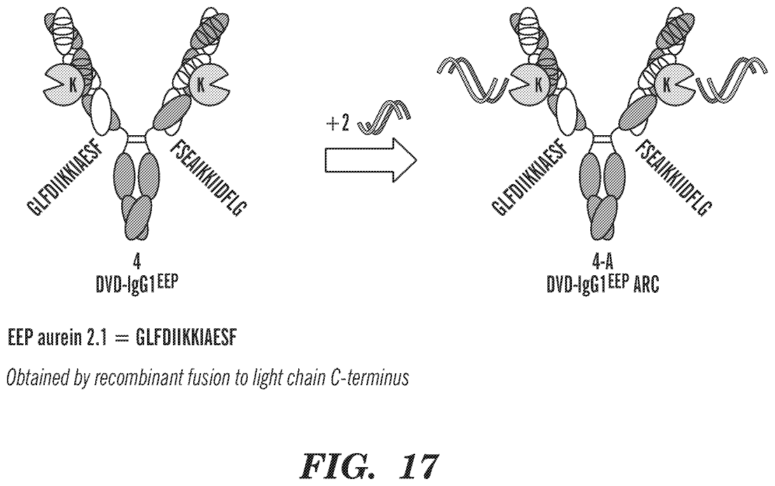

With the ARCs (8-13) in hand, knockdown of CTNNB1 mRNA was assessed using quantitative reverse transcription polymerase chain reaction (qRT-PCR) with R-actin as a housekeeping gene. Significant knockdown was observed against the MM cell line NCI-H929 for all the ARCs when treated at 90 nM for 72 h, though the BCMA-targeting ARCs (9 and 12) caused the highest level of silencing (FIG. 4A). To further investigate this knockdown, a dose response was performed using the BCMA-targeting ARCs (9 and 12) with an additional ARC (14) included as a negative control which was conjugated to an siRNA targeting human TTR (FIG. 2, compound 6), an irrelevant gene in this study (FIG. 9). Significant mRNA knockdown was observed at concentrations as low as 3 nM for both BCMA-targeting ARCs 9 and 12. ARC 14 did not cause any mRNA knockdown at the highest concentration (90 nM), as expected (FIG. 4B). To ensure that siRNA conjugation did not decrease binding of the outer variable domain to BCMA, surface-plasmon resonance (SPR) was performed using unconjugated anti-BCMA DVD-Fab or conjugated using the β-lactam hapten functionalized siRNA compounds targeting CTNNB1 (FIGS. 2, 4A, 4B and 5). The calculated equilibrium dissociation constants (K_{d}) were identical (FIGS. 15A-15C) before and after conjugation, indicating that the siRNA does not affect outer variable domain binding to BCMA.

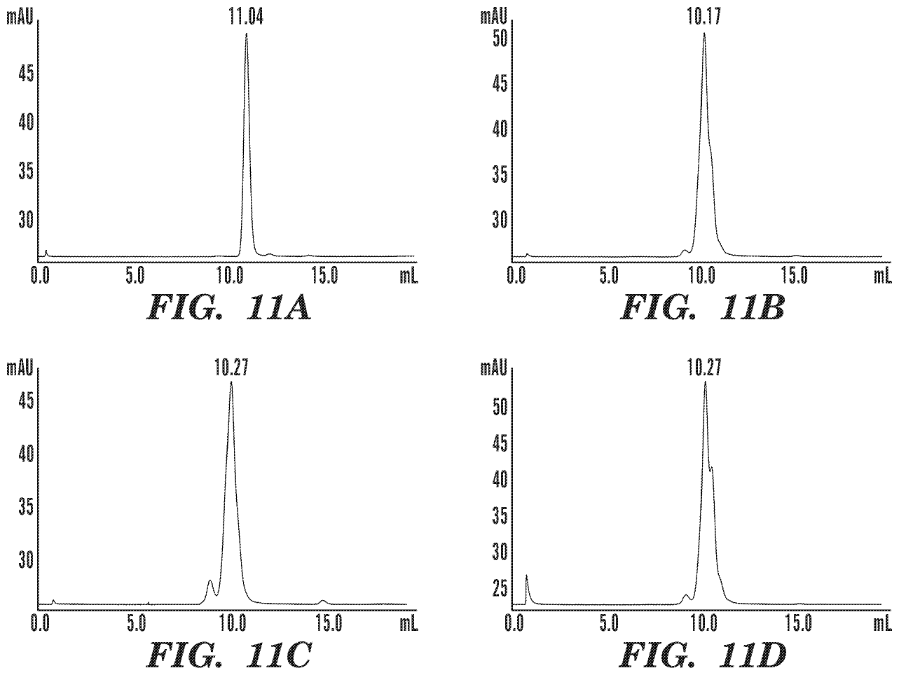

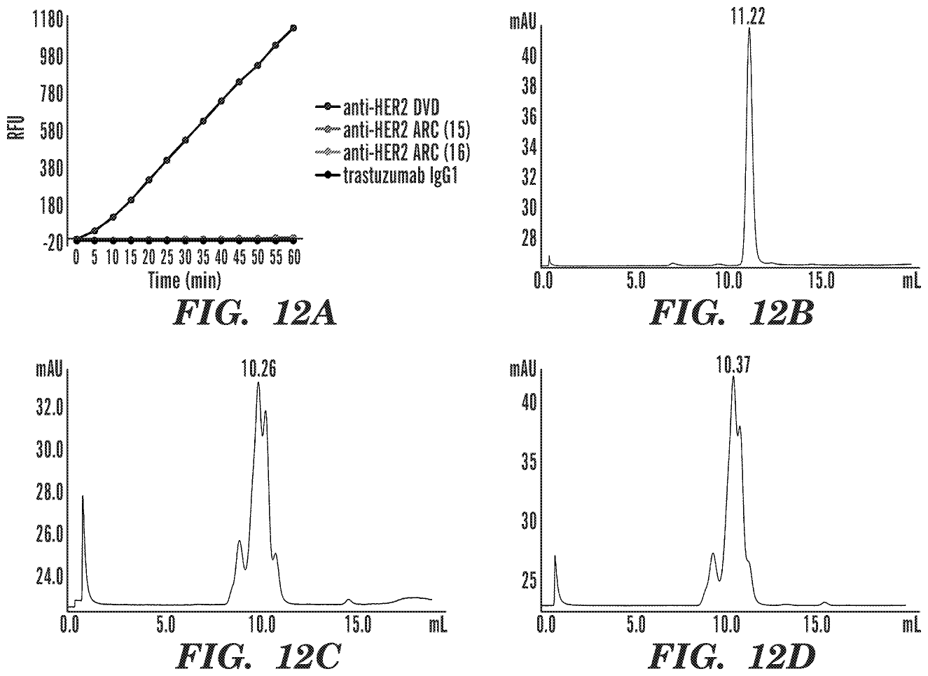

To further validate the BCMA-targeting ARCs, the conjugation reaction was optimized so only an equimolar ratio (1 eq with respect to each Lys residue) of siRNA was required for complete conjugation as indicated by complete loss of catalytic activity (FIGS. 10A-OB). The ARCs were purified using size-exclusion chromatography (SEC) to remove free siRNA compound and the increase in size indicated conjugation of siRNA to the DVD-IgGs (FIG. 11A-11D). To ensure the ARCs prepared in this manner had the same effect in vitro, CTNNB1 knockdown was compared to the unpurified ARCs prepared using a large excess of siRNA compound (FIG. 5). The CTNNB1 knockdown was identical between the two ARC samples. Furthermore, when an isotype control was prepared by conjugating the siRNA compounds to an anti-HER2 DVD (FIGS. 12A-12D) there was no significant knockdown as expected since NCI-H929 cells do not express HER2.¹⁸

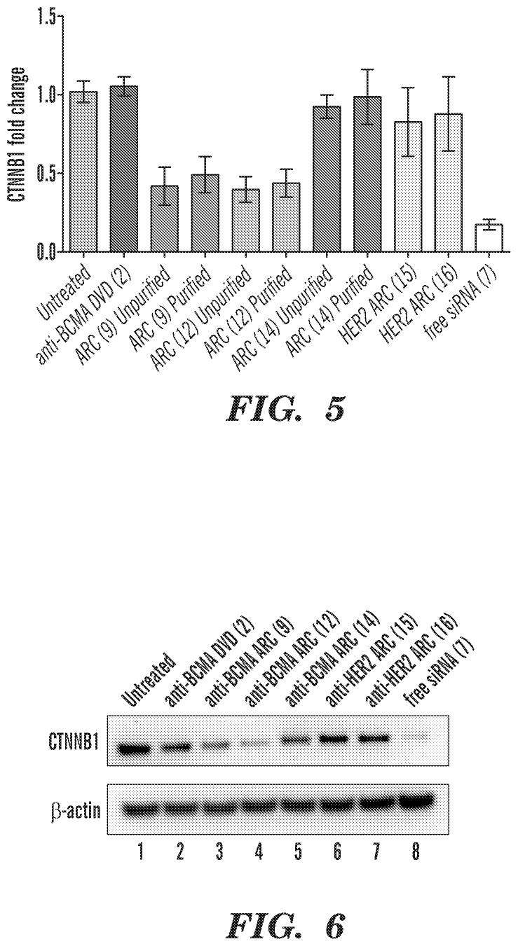

Lastly, to ensure the rt-qPCR mRNA knockdown correlated with protein knockdown, a western blot was performed after incubation with the ARCs at 90 nM for 7 days (FIG. 6). Significant CTNNB1 knockdown was observed for both BCMA-targeting ARCs (lanes 3 and 4), but there was not substantial knockdown for the unconjugated anti-BCMA DVD-IgG1 (lane 2), the anti-BCMA ARC targeting TTR (lane 5), or the anti-HER2 ARCs targeting CTNNB1 (lanes 6 and 7).

In summary, this work provides a method that generates site-specific ARCs using engineered DVDs that are mutation free and rely on rapid conjugation chemistry under neutral conditions. The generated ARCs retain binding towards the target antigen and successfully induce mRNA and protein knockdown in target cancer cells. Collectively, this strategy has several advantages over many antibody conjugation technologies and enables the selective delivery of siRNA for RNAi based strategies.

### Materials and Methods

Antibody cloning, expression, and purification: All variable domain sequences were based on published or patented amino acid sequences. All DVD-IgGs (1-3) were prepared as previously described.²⁸ DVD-IgG1s were prepared by linking the VH and VL of the targeting domain (anti-SLAMF7, BCMA, or CD138) to the VH and VL of h38C2 via a short (ASTKGP; the N-terminal 6 amino acids of human CH1) spacer. The desired sequences were synthesized as gBlocks (Integrated DNA Technologies) and expressed with human IgG1 heavy chain and κ light chain constant domains. The DVD expression cassettes were NheI/BamHI-cloned into a mammalian expression vector²⁹ and transiently transfected into Expi293F cells using Expifectamine according to the manufacturer's instructions (Life Technologies). After 5-7 days, the media was collected, filtered through a 0.22 micron filter, and purified using 1-mL HiTrap Protein A HP columns (GE Healthcare) in conjunction with an ÄKTA FPLC instrument (GE Healthcare). Yields were typically ˜40 mg/L. The purity of DVDs was confirmed by SDS-PAGE followed by Coomassie staining, and the concentration was determined by measuring the absorbance at 280 nm. The following protein sequences were prepared.

Anti-SLAMF7 DVD-IgG1 (1):
Light chain:
(SEQ ID NO: 22)
MPMGSLQPLATLYLLGMLVASVLADIQMTQSPSSLSASVGDRVTITCKASQDVGI
AVAWYQQKPGKVPKLLIYWASTRHTGVPDRFSGSGSGTDFTLTISSLQPEDVATY
YCQQYSSYPYTFGQGTKVEIKASTKGPELQMTQSPSSLSASVGDRVTITCRSSQSL
LHTYGSPYLNWYLQKPGQSPKLLIYKVSNRFSGVPSRFSGSGSGTDFTLTISSLQP
EDFAVYFCSQGTHLPYTFGGGTKVEIKRTVAAPSVFIFPPSDEQLKSGTASVVCLL
NNFYPREAKVQWKVDNALQSGNSQESVTEQDSKDSTYSLSSTLTLSKADYEKHK
VYACEVTHQGLSSPVTKSFNRGEC*
Heavy chain:
(SEQ ID NO:. 23)
MPMGSLQPLATLYLLGMLVASVLAEVQLVESGGGLVQPGGSLRLSCAASGFDFS
RYWMSWVRQAPGKGLEWIGEINPDSSTINYAPSLKDKFIISRDNAKNSLYLQMNS
LRAEDTAVYYCARPDGNYWYFDVWGQGTLVTVSSASTKGPEVQLVESGGGLV
QPGGSLRLSCAASGFTFSNYWMSWVRQSPEKGLEWVSEIRLRSDNYATHYAESV
KGRFTISRDNSKNTLYLQMNSLRAEDTGIYYCKTYFYSFSYWGQGTLVTVSSAST
KGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGA
Anti-BCMA DVD-IgG1 (2):
Light chain:
(SEQ ID NO: 20)
MPMGSLQPLATLYLLGMLVASVLADIQMTQSPSSLSASVGDRVTITCSASQDISN
YLNWYQQKPGKAPKLLIYYTSNLHSGVPSRFSGSGSGTDFTLTISSLQPEDFATYY
CQQYRKLPWTFGQGTKLEIKASTKGPELQMTQSPSSLSASVGDRVTITCRSSQSLL
HTYGSPYLNWYLQKPGQSPKLLIYKVSNRFSGVPSRFSGSGSGTDFTLTISSLQPE
DFAVYFCSQGTHLPYTFGGGTKVEIKRTVAAPSVFIFPPSDEQLKSGTASVVCLLN
NFYPREAKVQWKVDNALQSGNSQESVTEQDSKDSTYSLSSTLTLSKADYEKHKV
YACEVTHQGLSSPVTKSFNRGEC
Heavy chain:
(SEQ ID NO: 21)
MPMGSLQPLATLYLLGMLVASVLAQVQLVQSGAEVKKPGSSVKVSCKASGGTF
SNYWMHWVRQAPGQGLEWMGATYRGHSDTYYNQKFKGRVTITADKSTSTAY
MELSSLRSEDTAVYYCARGAIYDGYDVLDNWGQGTLVTVSSASTKGPEVQLVES
GGGLVQPGGSLRLSCAASGFTFSNYWMSWVRQSPEKGLEWVSEIRLRSDNYATH
YAESVKGRFTISRDNSKNTLYLQMNSLRAEDTGIYYCKTYFYSFSYWGQGTLVT
VSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTH
TCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSL
SLSPGA
Anti-CD138 DVD-IgG1 (3):
Light chain:
(SEQ ID NO: 18)
MPMGSLQPLATLYLLGMLVASVLADIQMTQSTSSLSASLGDRVTISCSASQGINN
YLNWYQQKPDGTVELLIYYTSTLQSGVPSRFSGSGSGTDYSLTISNLEPEDIGTYY
CQQYSKLPRTFGGGTKLEIKASTKGPELQMTQSPSSLSASVGDRVTITCRSSQSLL
HTYGSPYLNWYLQKPGQSPKLLIYKVSNRFSGVPSRFSGSGSGTDFTLTISSLQPE
DFAVYFCSQGTHLPYTFGGGTKVEIKRTVAAPSVFIFPPSDEQLKSGTASVVCLLN
NFYPREAKVQWKVDNALQSGNSQESVTEQDSKDSTYSLSSTLTLSKADYEKHKV
YACEVTHQGLSSPVTKSFNRGEC
Heavy chain:
(SEQ ID NO: 19)
MPMGSLQPLATLYLLGMLVASVLAQVQLQQSGSELMMPGASVKISCKATGYTF
SNYWIEWVKQRPGHGLEWIGEILPGTGRTIYNEKFKGKATFTADISSNTVQMQLS
SLTSEDSAVYYCARRDYYGNFYYAMDYWGQGTSVTVSSASTKGPEVQLVESGG
GLVQPGGSLRLSCAASGFTFSNYWMSWVRQSPEKGLEWVSEIRLRSDNYATHYA
ESVKGRFTISRDNSKNTLYLQMNSLRAEDTGIYYCKTYFYSFSYWGQGTLVTVSS
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLS
PGA
Anti-HER2 DVD-IgG1:
Light chain:
(SEQ ID NO: 24)
MDWTWRILFLVAAATGAHSDIQMTQSPSSLSASVGDRVTITCRASQDVNTAVAW
YQQKPGKAPKLLIYSASFLYSGVPSRFSGSRSGTDFTLTISSLQPEDFATYYCQQH
YTTPPTFGQGTKVEIKASTKGPELQMTQSPSSLSASVGDRVTITCRSSQSLLHTYG
SPYLNWYLQKPGQSPKLLIYKVSNRFSGVPSRFSGSGSGTDFTLTISSLQPEDFAV
YFCSQGTHLPYTFGGGTKVEIKRTVAAPSVFIFPPSDEQLKSGTASVVCLLNNFYP
REAKVQWKVDNALQSGNSQESVTEQDSKDSTYSLSSTLTLSKADYEKHKVYAC
EVTHQGLSSPVTKSFNRGEC*
Heavy chain:
(SEQ ID NO: 25)
MDWTWRILFLVAAATGAHSEVQLVESGGGLVQPGGSLRLSCAASGFNIKDTYIH
WVRQAPGKGLEWVARIYPTNGYTRYADSVKGRFTISADTSKNTAYLQMNSLRA
EDTAVYYCSRWGGDGFYAMDYWGQGTLVTVSSASTKGPEVQLVESGGGLVQP
GGSLRLSCAASGFTFSNYWMSWVRQSPEKGLEWVSEIRLRSDNYATHYAESVKG
RFTISRDNSKNTLYLQMNSLRAEDTGIYYCKTYFYSFSYWGQGTLVTVSSASTKG
PSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGA*

Flow cytometry: In a V-bottom 96-well plate (Corning), 100,000 cells per well were dispensed. The cells were washed with 200 μL flow cytometry buffer (PBS, 2% (v/v) FBS, 0.01% (w/v) NaN₃, pH 7.4), incubated with DVD-IgG1 or IgG1 (50 μL of a 20 nM solution in PBS) for 30 min on ice, washed with 200 μL ice-cold flow cytometry buffer, and stained with Alexa Fluor 647 conjugated polyclonal (Fab′)₂ donkey anti-human Fc (Jackson ImmunoResearch Laboratories) for 20 min on ice. After washing twice with 200 μL ice-cold flow cytometry buffer, the cells were analyzed using a Canto II Flow Cytometer (Becton-Dickinson). Data were analyzed using FlowJo software (Tree Star).

Antibody conjugation: Conjugations in FIG. 3 were performed in PBS (pH 7.4) after the antibodies were diluted to 15.7 μM (3.13 mg/mL). Next, 16.3 μL (2.75 mM H₂O solution of compound 4 shown in FIG. 2; 10 eq) or 19.8 μL (2.27 mM H₂O solution of compound 5 shown in FIG. 2; 10 eq) of β-lactam-siRNA was added to 900 μg of each antibody (1-3). Control anti-BCMA ARC 14 was prepared by diluting the anti-BCMA DVD to 4.08 mg/ml (20.4 μM), dispensing 2.0 mg, and adding 38 μL (2.59 mM H₂O solution of compound 6; 10 eq) of β-lactam-siRNA. The anti-HER2 ARCs (15 and 16) were prepared by diluting the anti-HER2 DVD to 4.47 mg/ml (22.4 μM), dispensing 2.0 mg, and adding 36 μL (2.75 mM H₂O solution of compound 4 or 5; 10 eq) of β-lactam-siRNA. The solutions were incubated for 2 h at room temperature (rt). For the optimized conjugation conditions, the anti-BCMA DVD was concentrated to 50.0 (10 mg/ml) using a 30-kDa cutoff centrifugal filter device (Millipore). Next, 2 eq of β-lactam-siRNA was added using a 2.75 mM solution of compound 4 or a 2.27 mM solution of compound 5. The solutions were incubated for 4 h at room temperature (rt). All conjugations were deemed complete by loss of catalytic activity using the methodol assay for which a portion of the crude reaction diluted to 1 μM in PBS was used. Upon completion, unreacted compound was removed by using a PD-10 desalting column (GE Healthcare). Protein containing fractions (Nanodrop A₂₈₀) were concentrated using a 4-ml 30-kDa cutoff centrifugal filter device (Millipore) and washed with ˜4 ml of PBS (×3). During the last wash, the samples were concentrated to a final volume of ˜250 μL. The concentration of the ARCs was determined using a BCA Assay kit (Thermo Fisher Scientific) according to the manufacturer's instructions with bovine gamma globulin (Thermo Fisher Scientific) as standard. For DVD-Fab conjugation, 2.0 mg of the anti-BCMA DVD-Fab was diluted to 7 mg/ml (100 μM) and 21.0 μL (2.75 mM H₂O solution of compound 4 or 5 shown in FIG. 2; 2 eq) of β-lactam-siRNA was added. The solutions were incubated for 4 h at room temperature (rt) and purified as described previously. All conjugates were stored in PBS at 4° C.

Surface Plasmon Resonance (SPR): SPR was used to determine the kinetic and thermodynamic parameters of the unconjugated anti-BCMA DVD-Fab and the anti-BCMA ARCs generated via conjugation with β-lactam hapten functionalized siRNA targeting CTNNB1 (FIG. 2, compounds 4 and 5). A Biacore X100 instrument was used with Biacore reagents and software (GE Healthcare). A mouse anti-human IgG C_{H}2 mAb was immobilized on a CM5 sensor chip using reagents and instructions supplied with the Human Antibody Capture Kit (GE Healthcare). hFc-hBCMA fusion protein was captured at a density not exceeding 1000 RU. Each sensor chip included an empty flow cell for instantaneous background depletion. All binding assays used 1×HBS-EP+running buffer (10 mM Hepes, 150 mM NaCl, 3 mM EDTA (pH 7.4), and 0.05% (v/v) Surfactant P20) and a flow rate of 30 μL/min. For affinity measurements, the anti-BCMA DVV-Fab or ARCs were injected at five different concentrations, one of which was tested in duplicate. The sensor chips were regenerated with 3 M MgCl₂ from the Human Antibody Capture Kit without any loss of binding capacity. Calculation of association (kₒₙ) and dissociation (k_{off}) rate constants was based on a 1:1 Langmuir binding model. The equilibrium dissociation constant (K_{d}) was calculated from k_{off}/kₒₙ.

Size-Exclusion Chromatography (SEC): SEC was performed on an AKTA FPLC system (GE Healthcare) equipped with a Superdex 200 10/300 GL column (GE Healthcare). For analytical runs, 30 μg of each sample was analyzed on the column with a flow rate of 0.5 mL/min PBS and the peaks were monitored at 280 nm. For purification, 1-2 mg of ARC samples were injected and the desired peak was collected at 280 nm.

Western Blotting: 500 μL of cells (4×10⁵ cell/mL, 200,000 cells per well) were dispensed in a 12-well cell culture dish. 500 μL of DVD-IgG1 or ARC solution (diluted with RPMI 1640 medium, supplemented with 10% FBS, 100 μg/mL streptomycin, and 100 U/mL penicillin) was immediately added at the appropriate concentration. Compound 7 (FIG. 2) was transfected using Lipofectamine RNAiMAX Transfection Reagent (Thermo Fisher) according to the manufacturer's instructions at a final siRNA concentration of 10 nM. 7 days later, the cells were washed with PBS and lysed using RIPA Lysis Buffer (Thermo Fisher) containing protease inhibitor cocktail (Thermo Fisher). The samples were diluted with 1×NuPAGE LDS sample buffer (Thermo Fisher) containing 2% (v/v) β-mercaptoethanol and boiled for 5 min before running on NuPAGE Novex 40%₆-12% Bis-Tris gels (Thermo Fisher). After transfer to a polyvinylidene difluoride membrane (Millipore) and blocking with 10% (v/v) western blot blocking solution (Sigma Aldrich, Cat #11921673001) in Tris-buffered saline containing 0.01% (v/v) Tween 20 (TBST), the membrane was incubated with 2 μg/mL anti-beta catenin 1 (CTNNB1) mouse IgG1 antibody (Biolegend, Clone: 12F7, Cat #844602) in 5% (v/v) western blot blocking solution (Sigma Aldrich, Cat #11921673001) in TBST at 4° C. overnight. The membrane was washed with TBST followed by incubation with a 1:5000 dilution (5% (v/v) western blot blocking in TBST) of HRP-conjugated goat anti-mouse IgG (Biolegend, Cat #405306) at room temperature for 3 h before washing with TBST and development using ECL Prime Western Blotting Detection Reagent (GE Healthcare). For R-actin staining, the membrane was incubated with a 1:10,000 dilution (5% (v/v) western blot blocking in TBST) of monoclonal mouse anti-β-actin peroxidase (Sigma Aldrich, Clone AC-15, Cat #A3854) at 4° C. overnight, washed with TBST, then imaged using ECL Prime Western Blotting Detection Reagent (GE Healthcare).

Synthesis of siRNAs 4, 5, 6, and 7: Efficient conjugation of the β-lactam moiety to siRNAs was achieved in the liquid-phase reaction of an excess of bis-β-lactam derivative 15 with β-catenin sense strand containing amino-moiety at the 5′-end (16, Q8u⋅a⋅cuguUgGAUugauucga⋅a⋅a (SEQ ID NO: 36)) (FIG. 7), β-catenin sense strand containing amino-moiety at the 3′-end (17) (u⋅a⋅cuguUgGAUugauucgaaaL8 (SEQ ID NO: 37)), where lower case letters stand for 2′-OMe nucleosides, upper-case letters in italics stand for 2′-deoxy-2′-F nucleosides, L8 stands for 3′-aminoprolinol linker, and a thick dot between nucleosides stands for phosphorothioate linkages), and negative control (hTTR) sense strand (18) (u⋅g⋅ggauUuCAUguaaccaagaL8 (SEQ ID NO: 38). Thus the corresponding β-lactam conjugated sense strands 19 (Q321u⋅a⋅cuguUgGAUugauucga⋅a⋅a (SEQ ID NO: 39)), 20 (u⋅a⋅cuguUgGAUugauucgaaaQ321′ (SEQ ID NO: 40)) and 21 (u⋅g⋅ggauUuCAUguaaccaagaQ321′ (SEQ ID NO: 41)) were obtained, where Q321 and Q321′ are as follows:

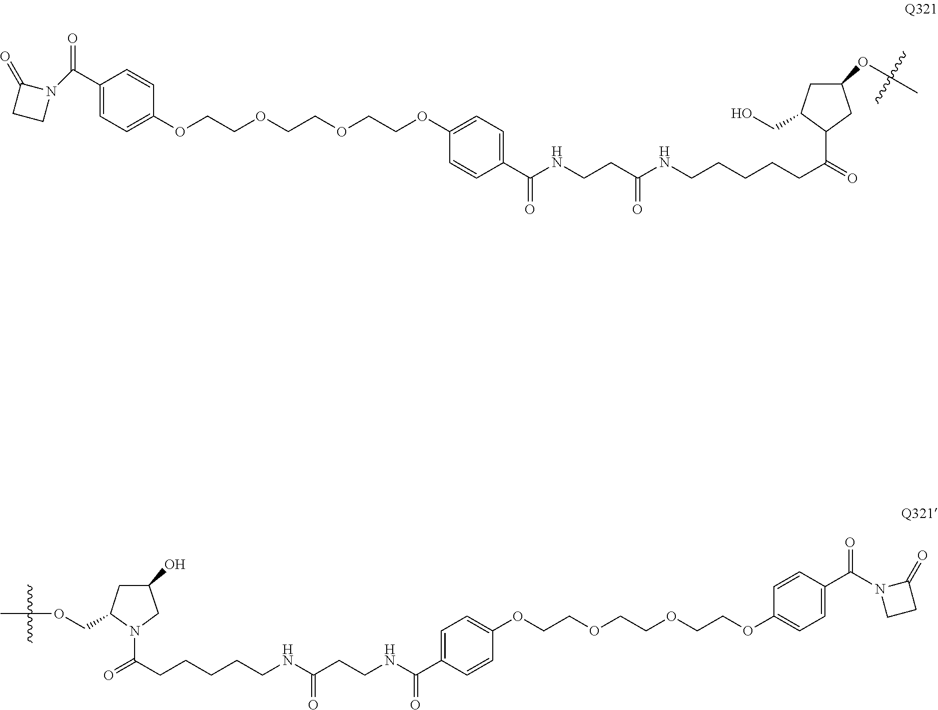 [SMILES: *O[C@H]1CC(C(=O)CCCCCNC(=O)CCNC(=O)c2ccc(OCCOCCOCCOc3ccc(C(=O)N4CCC4=O)cc3)cc2)[C@H](CO)C1 ; *OC[C@@H]1C[C@@H](O)CN1C(=O)CCCCCNC(=O)CCNC(=O)c1ccc(OCCOCCOCCOc2ccc(C(=O)N3CCC3=O)cc2)cc1]

Typical procedure: A solution of 16 (24.4 mg, 33 NM) in water (1 mL) was added to a solution of 15 (32.7 mg, 660 μM) in acetonitrile (1 mL) followed by addition of 50 μL of 10% solution of triethylamine in acetonitrile. The mixture was kept at rt with occasional swirling for 3 h, and 6 drops of 10% (v/v) solution of acetic acid in acetonitrile was added. The mixture was diluted with water (7 mL) and DCM (4 mL) was added. After the extraction, the aqueous phase was separated, washed with DCM (5 mL×2) and stirred in vacuum (up to 10 mbar) for complete evaporation of organic solvents. The aqueous solution of β-lactam conjugate 19 thus obtained was further used for low temperature duplex annealing with the corresponding antisense strand (22), where VP(Tam) stands for 5′-(E)-vinylphosophonate-2′-N-methylacetamide 5-methyl uridine^{Error! Bookmark not defined.}) to afford duplex 4 (FIG. 2).

Duplex 5 (FIG. 2) was prepared analogously from 15 and 17 followed by annealing with 22. Negative control (hTTR) duplex 7 (FIG. 2) was prepared analogously from 15 and 18 followed by annealing with the corresponding antisense strand (23). Unconjugated control siRNA 6 was prepared by annealing of 17 with 22. For details on automated synthesis of oligonucleotides 16, 17, 18, 22, 23, and analytical data for all oligonucleotides 16-23 see below.

Compounds 4-7 were dissolved in Ambion nuclease-free water (Thermo Fisher Scientific) to a working concentration of 2.27 or 2.75 mM and stored as aliquots at −80° C.

Catalytic activity assay: Catalytic activity was analyzed using methodol^{Error!Bookmark not defined.} as described previously.³⁰ DVD-IgGs, IgG1s, and ARCs were diluted to 1 μM in PBS (pH 7.4) and dispensed in 98-μl aliquots into a 96-well plate in triplicate. Then, 2 μL of 10 mM methodol in ethanol was added and the fluorescence was assessed immediately using a SpectraMax M5 instrument (Molecular Devices) with SoftMax Pro software, a wavelength of excitation (λₑₓₜ) set to 330 nm, a wavelength of emission (λₑₘ) set to 452 nm, and starting at 0 min using 5-min time points. The signal was determined by normalizing to 98 μL PBS with 2 μL of the methodol solution added.

Cell lines: Human MM cell lines U-266, NCI-H929, and RPMI-8226 were obtained from American Type Culture Collection (ATCC) and cultured in RPMI 1640 medium, supplemented with 10% FBS, 100 μg/mL streptomycin, and 100 U/mL penicillin at 37° C. in an atmosphere of 5% CO₂ and 100% humidity. Expi293F cells were grown in Expi293 Expression media (Life Technologies, Carlsbad, Calif.) at 37° C. in an atmosphere of 8% CO₂ and 100% humidity.

Cell treatment, RNA extraction, and quantitative real-time PCR (qPCR): 500 μL of cells (4×10⁵ cell/ml, 200,000 cells per well) were dispensed in a 12-well cell culture dish. 500 μL of DVD-IgG1 or ARC solution (diluted with RPMI 1640 medium, supplemented with 10% FBS, 100 μg/mL streptomycin, and 100 U/mL penicillin) was immediately added at the appropriate concentration. Compound 7 (FIG. 2) was transfected using Lipofectamine RNAiMAX Transfection Reagent (Thermo Fisher) according to the manufacturer's instructions at a final siRNA concentration of 10 nM. 72 h later, extraction of RNA from cell lysates was performed using Qiagen RNeasy kit (Qiagen) followed by cDNA synthesis of 1 μg DNase-digested RNA, using the Maxima First-Strand cDNA Synthesis Kit for quantitative RT-PCR (Invitrogen) according to the manufacturer's instructions. Quantitative PCR of the synthesized cDNA was conducted using SYBR Green PCR Master Mix (Applied Biosystems) according to the manufacturer's protocol. qRT-PCR reactions were performed on StepOnePlus Real-Time PCR System (Applied Biosystems) and analyzed using StepOne Software v2.2.2. All measurements were conducted three times using biological duplicates or triplicates and standardized to the levels of β-actin. Relative changes in gene expression were calculated according to the 2^{-ΔΔCT} algorithm.³¹

Primer sequences:³²
β-actin
Forward
(SEQ ID NO: 42)
5′ CCTGTACGCCAACACAGTGC 3′
Reverse
(SEQ ID NO: 43)
5’ ATACTCCTGCTTGCTGATCC 3′
β-catenin
Froward
(SEQ ID NO: 44)
5′ AAAATGGCAGTGCGTTTAG 3′
Reverse
(SEQ ID NO: 45)
5′ TTTGAAGGCAGTCTGTCGTA 3′

Synthesis of “non-cleavable” bis-β-lactam linker (15): Exemplary non-cleavable bis-β-lactam linker (15) was synthesized as shown in Scheme 1.

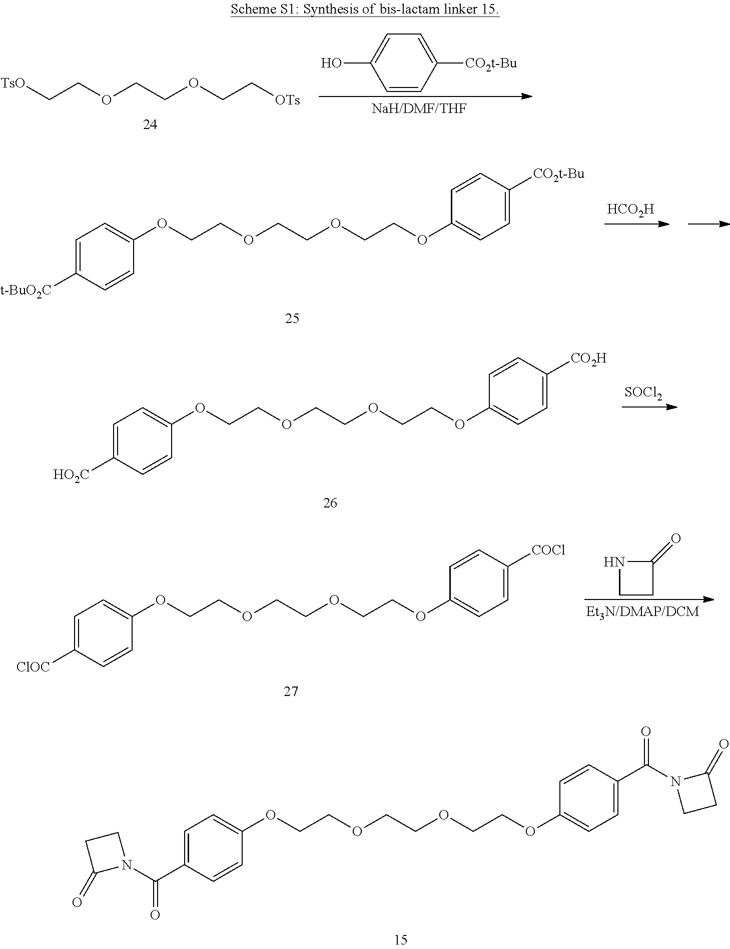

Synthesis of 2,2′-[1,2˜Ethanediylbis(oxy)]bis-,1,1′-bis(4-(tert-butoxycarbonyi) phenoxy)ethanol (25): To a stirred solution of purified by crystallization commercial bis-tosylate 24 (>98% purity, 3.90 g, 8.5 mmol) and tert-butyl 4-hydroxybenzoate (3.69 g, 19 mmol) in a mixture of anhyd. DMF (30 mL) and THF (30 mL) was added 60% oil dispersion of NaH (0.80 g, 20 mmol) under Ar atmosphere at −78° C. and the mixture was allowed to warm in bath up to 0° C. The cooling bath was replaced for heating bath, the mixture was heated at 40° C. for additional 5 h, cooled to 0° C., and quenched by addition of saturated aq. NH₄Cl followed by AcOEt and minimum water to dissolve inorganic salts. The organic phase was separated, washed twice with 5% aq. NaCl, saturated NaCl, and dried over anhyd. Na₂SO₄. The solvent was evaporated in vacuum to afford 4.92 g of crude residue. Chromatography of the residue over a flash column of silica gel with isocratic 30% AcOEt in hexanes gave 3.12 g (73%) of 25 as white solid. ¹H NMR (400 MHz, DMSO-d₆): δ 1.51 (s, 18H); 3.60 (s, 4H); 3.75 (dd, J=3.0, 4.6 Hz, 4H); 4.13 (dd, J=3.2, 4.5 Hz, 4H); 6.99 (d split, J=9.0 Hz, 4H); 7.81 (d split, J=9.0 Hz, 4H). ¹³C NMR (126 M1 Hz, DMSO-d₆): δ 27.8, 67.4; 68.7; 69.9; 80.1; 114.2; 123.6; 131.0; 162.0; 164.6. HRMS: [M+Na]+calc. for C₂₈H₃₈O₈Na⁺, 525.2459, found: 525.2444.

Synthesis of 2,2′-[1, 2-Ethanediylbis(oxy)]bis-, 1,1′-bis(4-carboxyphenoxy)ethanol (26): A solution of 25 (3.10 g, 6.2 mmol) in HCO₂H (98%, 60 mL) was stirred at rt for 4 h, and a mixture of toluene (60 mL) and diethyl ether (60 mL) was added. The suspension was stirred at rt overnight, filtered, white crystalline solid was washed thoroughly with toluene-diethyl ether (1:1) mixture, and dried to afford 2.41 g (100%) of crude 26. The latter was recrystallized from 100 mL of dry EtOH to afford 2.15 g (89%) of pure 26. ¹H NMR (400 MHz, DMSO-d₆): δ 3.60 (s, 4H); 3.75 (dd, J=3.0, 4.6 Hz, 4H); 4.14 (dd, J=3.0, 4.5 Hz, 4H); 6.99 (d split, J=8.9 Hz, 4H); 7.86 (d split, J=8.8 Hz, 4H); 12.6 (s, 2H). ¹³C NMR (126 MHz, DMSO-d₆): δ 67.4; 68.8; 69.9; 114.3; 123.0; 131.3; 162.1; 163.0. MS (ES+APCI) neg. scan [M−H]⁻=calc. exact 389.1236; found: 389.1.

Synthesis of 2,2′-[1,2-Ethanediylbis(oxy)]bis-1,1′-bis(4-[chlorocarbonyl]phenoxy)ethanol (27):

A suspension of bis-acid 26 (1.58 g, 4.1 mmol) in anhyd. CHCl₃ (35 mL) was heated at reflux in the presence of SOCl₂ (2.7 mL, 37 mmol) until complete dissolution of 26 (8 h). The mixture was cooled to rt under Ar atmosphere and diluted with anhyd. toluene (30 mL), the solvents were evaporated in vacuum followed by drying in high vacuum to afford 27 (1.73 g, 100%) as gradually crystallizing colorless oil. ¹H NMR (400 MHz, CDCl₃): 3.75 (s, 4H); 3.89 (dd, J=3.5, 4.8 Hz, 4H); 4.21 (dd, J=3.5, 4.6 Hz, 4H); 6.96 (d split, J=9.1 Hz, 4H); 8.05 (d split, J=9.1 Hz, 4H). ¹³C NMR (126 MHz, DMSO-d₆): δ 68.1; 69.6; 71.1; 114.9; 125.8; 134.13; 164.8; 167.3.

Synthesis of Triethylene glycol bis(1-[4-oxybenzoyl)]-2-azetidinone) (15): The synthesis of bis-2-azetidinone derivative 15 was performed by analogy to published procedure of triethylamine-catalyzed coupling of 2-azetidinones with acyl chlorides.³³ A solution of commercial 2-azetidinone (0.57 g, 8 mmol) in anhyd. DCM (20 mL) was cooled to −78° C. under Ar atmosphere, and DMAP (0.1 g, 0.8 mmol) was added. A solution of dichloride 27 (1.72 g, 4 mmol) in anhyd. DCM (10 nL) was added via cannula for 5 min followed by dropwise addition of triethylanine (1.0 mL, 7.2 mmol) for 10 min. The mixture was stirred at −78° C. for 1 h, the cooling bath was removed, the mixture was stirred at rt for 23 h, diluted with DCM (10 mL) and quenched by addition of water (50 mL). The organic phase was separated, washed consecutively with water (50 mL), saturated NaCl, and dried over anhyd. Na₂SO₄. The solvent was evaporated in vacuum to afford 1.73 g of crude residue. Chromatography of the residue over a flash column of silica gel with isocratic 80% AcOEt in hexanes gave 1.01 g (51%) of 15 as a white solid. ¹H NMR (400 MHz, CD3CN): 3.02 (t, J=5.6 Hz, 4H); 3.64 (t, J=5.5 Hz, 4H); 3.65 (s, 4H); 3.79 (dd, J=3.1, 4.6 Hz, 4H); 4.16 (dd, J=3.2, 4.5 Hz, 4H); 6.97 (d split, J=9.0 Hz, 4H); 7.91 (d split, J=9.0 Hz, 4H). ¹³C NMR (126 MHz, CD3CN): δ 35.7; 37.6; 68.8; 70.2; 71.4; 114.9; 125.9; 132.9; 163.7; 165.9; 166.2. MS (ES+APCI) pos. scan [M+H]+=calc. exact 497.1924; found: 497.2. HRMS: [M+H]⁺ calc. for C₂₆H₂₉N₂O₈, 497.1924, found: 497.1945.

Synthesis of “cleavable” bis-β-lactam linker (11): Exemplary cleavable bis-β-lactam linker was synthesized as shown in Scheme 2.

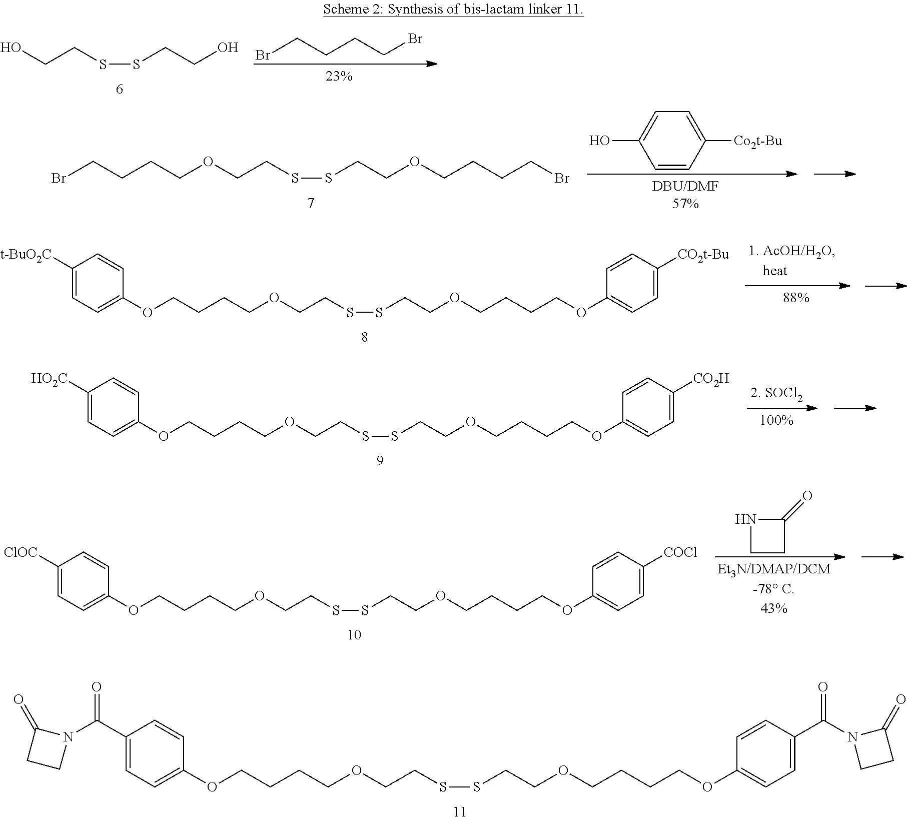

Synthesis of 1,16-Dibromo-5,12-dioxa-8,9-dithiahexadecane (7): To a stirred solution of disulfide 6 (2.4 mL, 20 mmol) and 1,4-dibromobutane (14.3 mL, 120 mmol) in a mixture of anhyd. DMF (50 mL) and THF (50 mL was added 60% oil dispersion of NaH (2.40 g, 60 mmol) under Ar atmosphere at −78° C. and the mixture was allowed to warm in bath up to it overnight. The solvents were removed in vacuum and the residue was partitioned between AcOEt (100 mL) and 5% aq. NaCl (100 nL), the organic phase was separated, washed with 5% aq NaCl, saturated NaCl, and dried over anhyd. Na₂SO₄. Flash chromatography of the crude residue (15.0 g) with 15% of AcOEt in hexanes gave 1.92 g (23%) of 7 as colorless liquid. ¹H NMR (500 MHz, DMSO-d₆): δ 1.57-1.64 (m, 4H); 1.80-1.89 (m, 4H); 2.89 (t, J=6.3 Hz, 4H); 3.42 (t, J=6.3 Hz, 4H); 3.54 (t, J=6.8 Hz, 4H); 3.59 (t, J=6.3 Hz, 4H). ¹³C NMR (126 MHz, DMSO-d₆): δ 28.4; 29.9; 35.6; 38.6; 68.7; 69.7. MS (ES+APCI) pos. scan [M+Na]+=calc. 447.2; found: 447.0.

Synthesis of bis-1,16-(4-(tert-Butoxycarbonyl)phenoxy)-5,12-dioxa-8,9-dithiahexadecane (8): DBU (1.4 mL, 9.5 mmol) was added to a suspension of dibromo compound 7 (1.90 g, 4.5 mmol) and tert-butyl 4-hydroxybenzoate (2.13 g, 11 mmol) in anhyd. DMF (15 mL) under Ar atmosphere, the mixture was heated at 40° C. for 60 b, cooled to rt and quenched by addition of 1:1 solution of sat NH₄Cl and water. The product was extracted with AcOEt, the organic phase was washed twice with 5% aq. NaCl, saturated NaCl, and dried over anhyd. Na₂SO₄. Flash chromatography of the crude residue (2.19 g) with 15% of AcOEt containing 1% of triethylamine in hexanes gave 1.68 g (57%) of 8. ¹H NMR (500 MHz, DMSO-d₆): δ 1.51 (s, 18H); 1.59-1.66 (m, 4H); 1.72-1.79 (m, 4H); 2.88 (t, J=6.3 Hz, 4H); 3.44 (t, J=6.4 Hz, 4H); 3.59 (t, J=6.3 Hz, 4H); 4.02 (t, J=6.5 Hz, 4H); 6.96 (d split, J=9.0 Hz, 4H); 6.96 (d split, J=9.0 Hz, 4H); 7.81 (d split, J=6.2 Hz, 4H). ¹³C NMR (126 MHz, DMSO-d₆): δ 25.4; 25.6; 27.8; 38.0; 67.5; 68.1; 69.6; 80.0; 114.1; 123.4; 131.0; 162.2; 164.6. MS (ES+APCI) pos. scan [M+Na]+=calc. 673.2839; found: 673.3.

Synthesis of bis-1,16-(4-Carboxyphenoxy)-5,12-dioxa-8,9-dithiahexa decane (9): A solution of bis-t-butyl ester 8 (1.67 g, 2.6 mmol) in AcOH (30 mL) and water (3 mL) was stirred at 100° C. for 8 h, the heating bath was removed and ACN (90 mL) was added to hot solution. Crystallization of 9 started soon, the suspension was stirred at rt overnight, the precipitate was filtered, washed twice with ACN and dried in air flow to afford 1.23 g (88%) of pure 9 as an white solid, ¹H NMR (400 MHz, DMSO-d₆): δ 1.58-1.68 (m, 4H); 1.70-1.81 (m, 4H); 2.89 (t, J=6.3 Hz, 4H); 3.44 (t, J=6.3 Hz, 4H); 3.60 (t, J=6.3 Hz, 4H); 4.03 (t, J=6.4 Hz, 4H); 6.98 (d split, J=9.0 Hz, 4H); 7.86 (d split, J=9.0 Hz, 4H); 12.57 (s, 2H). ¹³C NMR (126 MHz, DMSO-d₆): δ 25.4; 25.6; 38.0; 67.5; 68.1; 69.6; 114.2; 122.8; 131.3; 162.2; 167.0. MS (ES+APCI) neg. scan [M−H]⁻=calc. 537.1622; found: 537.2.

Synthesis of bis-1,16-(4-[Chlorocarbonyl]phenoxy)-5J2-dioxa-8,9-dithiahexadecane (10): A suspension of bis-acid 9 (1.22 g, 2.3 mmol) in anhyd. chloroform (35 mL) containing SOCl₂ (3.6 mL, 50 mmol) was heated under reflux in Ar atmosphere for 5 b, cooled to rt, and anhyd. toluene (50 mL) was added. The solvents were evaporated in vacuum, the residue was redissolved in anhyd. toluene (50 mL), and the solvent was removed in vacuum to afford 1.33 g (quant) of 10 as a colorless oil ¹H NMR (500 MHz, CDCl₃): δ 1.72-1.80 (m, 4H); 1.85-1.94 (m, 4H); 2.88 (t, J=6.5 Hz, 4H); 3.53 (t, J=6.3 Hz, 4H); 3.68 (t, J=6.5 Hz, 4H); 4.07 (t, J=6.4 Hz, 4H); 6.94 (d, J=8.9 Hz, 4H); 8.04 (d, J=8.9 Hz, 4H). ¹³C NMR (126 MHz, CDCl₃): δ 26.1; 26.3; 38.9; 68.5; 69.2; 70.7; 114.8; 125.4; 134.2, 165.1; 167.3.

Synthesis of bis-1,16-(1-[4-Oxybenzoyl)]-2-azetidinone)5,12-dioxa-8,9-dithiahexadecane (11): A solution of commercial 2-azetidinone (0.39 g, 5.5 mmol) in anhyd. DCM (15 mL) was cooled to −78° C. under Ar atmosphere, aid DMAP (61 mg, 0.5 mmol) was added. A solution of dichloride 10 (1.44 g, 2.5 mmol) in anhyd. DCM (6 mL) was added via cannula for 5 min followed by dropwise addition of triethylamine (0.63 mL, 4.5 mmol) for 10 min. The mixture was stirred at −78° C. for 1 h, allowed to slowly warm up to rt in bath overnight, diluted with DCM (20 mL) and quenched by addition of water (50 nL). The organic phase was separated, washed consecutively with water (80 mL), saturated NaCl, and dried over anhyd. Na₂SO₄. The solvent was evaporated in vacuum to afford 1.75 g of crude residue. Chromatography of the residue over a flash column of silica gel with isocratic 10% diethyl ether in DCM gave 0.69 g (43%) of 11 as slowly crystalizing oil. ¹H NMR (400 MHz, DMSO-d₆): 1.58-1.68 (m, 4H); 1.71-1.81 (m, 4H); 2.89 (t, J=6.2 Hz, 4H); 3.07 (t, J=5.6 Hz, 4H); 3.45 (t, J=6.3 Hz, 4H); 3.61 (q, J=6.1 Hz, 8H); 4.05 (t, J=6.4 Hz, 4H); 7.00 (d split, J=9.0 Hz, 4H); 7.87 (d split, J=9.0 Hz, 4H). ¹³C NMR (126 MHz, DMSO-d₆): δ 25.4; 25.6; 34.6; 36.7; 38.0; 67.6; 68.1; 69.6; 113.8; 124.0; 131.7; 162.4; 164.6; 164.7. MS (ES+APCI) pos. scan [M+H]+=calc. 645.2299; found: 645.3.

Automated synthesis of single strands 16, 17, 18, 22, and 23: Sense and antisense strands (Table 2) were synthesized on an Äkta Oligopilot 100 using commercially available 5′-O-(4,4′-dimethoxytrityl)-2′-deoxy-2′-fluoro-,5′-O-(4,4′-dimethoxytrityl)-2′-O-(tert-butyldimethyl silyl)-, and 5′-O-(4,4′-dimethoxytrityl)-2′-O-methyl-3′-O-(2-cyanoethyl-N,N-diisopropyl) phosphoramidite monomers of uridine, 4-N-acetylcytidine, 6-N-benzoyladenosine, and 2-N-isobutyrylguanosine using standard modified versions of the preinstalled solid-phase RNA synthesis cycles on the Äkta Oligopilot.

For oligonucleotide 16, the inventors placed a N-(aminocaproyl)prolinol-4-phosphate modification at the 5′ end, where the amine was protected with a TFA group. The oligo was cleaved and deprotected in 50/50 v/v solution of AMA: Aq. ammonia (30% wt/v) and Aq. methylamine (40% wt/v) for 3 hrs at R.T. Oligonucleotides 17 and 18 had a similar N-(aminocaproyl)-4-hydroxyprolinol modification at the 3′ terminus, which was introduced on the CPG support. This was also TFA protected and cleaved the same way.

Oligonucleotides 22 and 23 have a 5′-(E)-vinylphosophonate 2′ N-methylacetamide 5′-methyluridine monomer, which was introduced and cleaved using previously published protocols.³⁴

After cleavage all oligonucleotides were filtered through a 0.45-μm filter to remove solid residues, and the support was rinsed with water (1.5 mL/μmol of solid support). The crude ligand-conjugated and unconjugated oligonucleotides were purified by anion-exchange high-performance liquid chromatography (IEX-HPLC) with TSK-Gel Super Q-5PW support (TOSOH Corp.) using a linear gradient of 22-42% buffer B over 130 min with 50 mL/min flow rate (Buffer A: 0.02 M Na₂HPO₄ in 10% CH₃CN, pH 8.5 and buffer B: buffer A plus 1 M NaBr). All single strands were purified to >85% HPLC (260 nm) purity and then desalted by size exclusion chromatography on an AKTA Prime chromatography system using an AP-2 glass column (20×300 mm, Waters) custom-packed with Sephadex G25 (GE Healthcare), eluted with sterile nuclease-free water. The isolated yields for the oligonucleotides were calculated based on the respective ratios of measured to theoretical 260 nm optical density units. The integrities of the purified oligonucleotides were confirmed by LC-MS (Table 2) and by analytical IEX HPLC. Equimolar amounts of complementary sense and antisense strands were mixed and annealed by heating in a water bath at 95° C. for 5 min and cooling to room temperature to obtain the desired siRNAs. The siRNA samples were analyzed for purity, endotoxin, and osmolality, and the observed values were within the allowed range for the concentration tested.

TABLE 2
Single strands 16-18, 22, and 23

| Sequence | | Mass | |
|---|---|---|---|
| ID | Sequence (5′-3′)^{[a]} | Calc. | Observed |
| 16 (SS) | Q8•u•a•-cuguUgGAUugauucga•a•a (SEQ ID NO: 38) | 7301.943 | 7300.14 |
| 17 (SS) | u•a•cuguUgGAUugauucgaaaL8 (SEQ ID NO: 38) | 7269.813 | 7268.25 |
| 18 (SS) | u•g•ggauUuCAUguaaccaagaL8 (SEQ ID NO: 39 | 7291.868 | 7289.65 |
| 22 (AS) | VP(Tam)•U•ucgAaUCaaucCaAcagua•g•c (SEQ ID NO: 43) | 7737.128 | 7735.98 |
| 23 (AS) | VP(Tam)CuugGuuAcaugAaAuccca•u•c (SEQ ID NO: 44) | 7670.953 | 7669.53 |

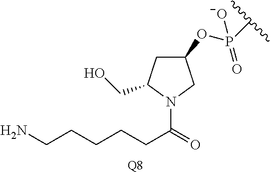

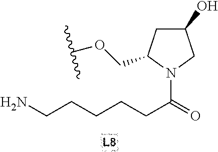 [SMILES: *OC[C@@H]1C[C@@H](O)CN1C(=O)CCCCCN]

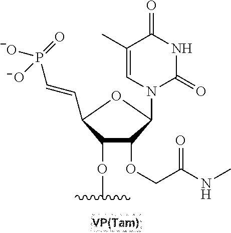

^{[a]}Italicized upper case and normal lower case letters indicate 2′-deoxy-2′-fluoro, and 2′-O-methyl sugar modifications, respectively, to adenosine (A), cytidine (C), guanosine (G) and uridine (U);
•indicates phosphorothioate (PS) linkage.

Analytical data for β-lactam-conjugated single strands 19, 20, and 21: Reverse phase LC/MS analyses of b-lactam conjugates 19, 20, and 21 was carried out using an Agilent 6130 Quadrupole LC/MS connected to an Agilent 1260 Infinity HPLC system. Standard oligonucleotide LC conditions were used with a column temperature of 60° C. and 95 mM of HFIP and 16 mM TEA in water as a mobile phase A and MeOH as a mobile phase B. It was found that in these conditions p3-lactam ring undergoes complete cleavage by all presenting nucleophiles affording water, MeOH, and HFIP adducts correspondingly (FIGS. 8A and 8B, and Scheme 3). Small amounts of the target products could be observed by eluting at low temperature, however even in these conditions major amounts of cleavage products presented. Representative example of a typical chromatogram (compound 19) are shown in FIGS. 8A and 8B.

For reliable analysis of the reaction course and purity of the product we developed aliquot pretreatment protocol by treatment with excess of butylamine for 5 min followed by running the HPLC. The butylamine adduct is stable in chromatography conditions and gives a single peak of the product, and all impurities existing in a β-lactam derivative before the pretreatment should manifest on an HPLC chromatogram. The results suggest a minimum purity of 85% of the product before pretreatment with the lactam ring hydrolysis by-product as main impurity that apparently formed during the reaction of 15 with 16. In some cases, the inventors observed an apparent shoulder before the main peak of the butylamine adduct that showed the same mass of the adduct, and which formation was attributed to change in composition of the cationic butylamine bound to siRNA during a run.

MALDI analysis of conjugates 19, 20, and 21: MALDI mass spectra were collected using a Bruker Microflex LRF MALDI mass spectrometer. Samples were analyzed in the linear positive ion mode, with 500 laser shots collected at random across each sample spot and summed using the automated sample collection mod.

MALDI Sample Preparation: After the reaction of sense strands 16, 17, and 18 with bis-β-lactam compound 15, the solutions of siRNA conjugates 19, 20, and 21 in water in concentration of ˜3 mg/mL were used for MALDI analysis. Matrix solutions were prepared as saturated solutions and used within 1 day. A 50 mg/mL solution of diammonium citrate in deionized water and a solution of 10 mg of THAP in 1.0 mL acetonitrile-deionized water (1:1, v/v) were separately prepared, combined in 1:8 ratio, and vortexed to obtain the matrix prior to analysis.

Sample analysis: 1.0 μL of a solution of β-lactam siRNA conjugate was aliquoted into a microcentrifuge tube containing 9 μL of the matrix and mixed well. 1-2 μL of the sample/matrix solution was loaded onto a stainless steel target plate and allowed to dry at ambient temperature and pressure before MALDI sample analysis. Masses obtained by MALDI analysis of compounds 19, 20, and 21 were as follows:

Conjugate 19: MS calc. for [M+H⁺] 7799.47, observed 7800.79; [M+Na⁺] calc. 7821.45, observed 7823.56. Conjugate 20: MS calc. for [M+H⁺] calc. 7768.35, observed 7769.62; [M+Na⁺] calc. 7790.33, observed 7791.84; [M] as Na salt+Na⁺ ([M−H⁺+2Na⁺]) calc. 7812.31, observed 7813.70. Conjugate 21: MS calc. for [M+H⁺] 7790.41, observed 7792.21.

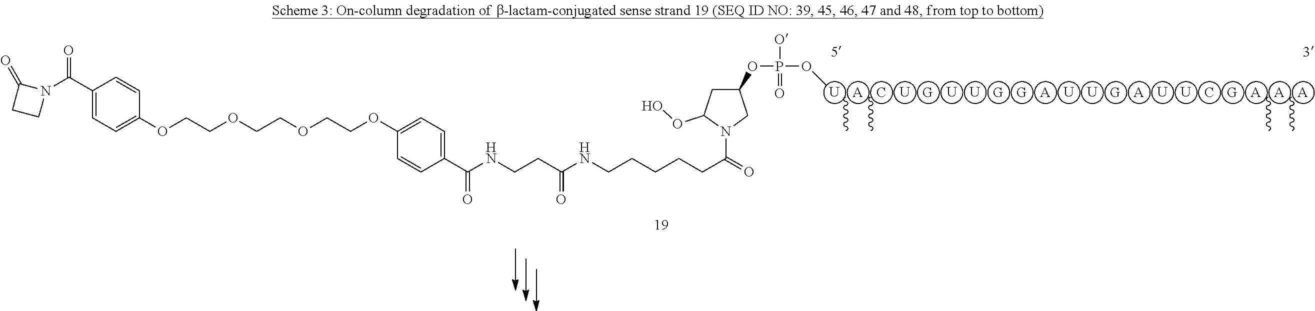

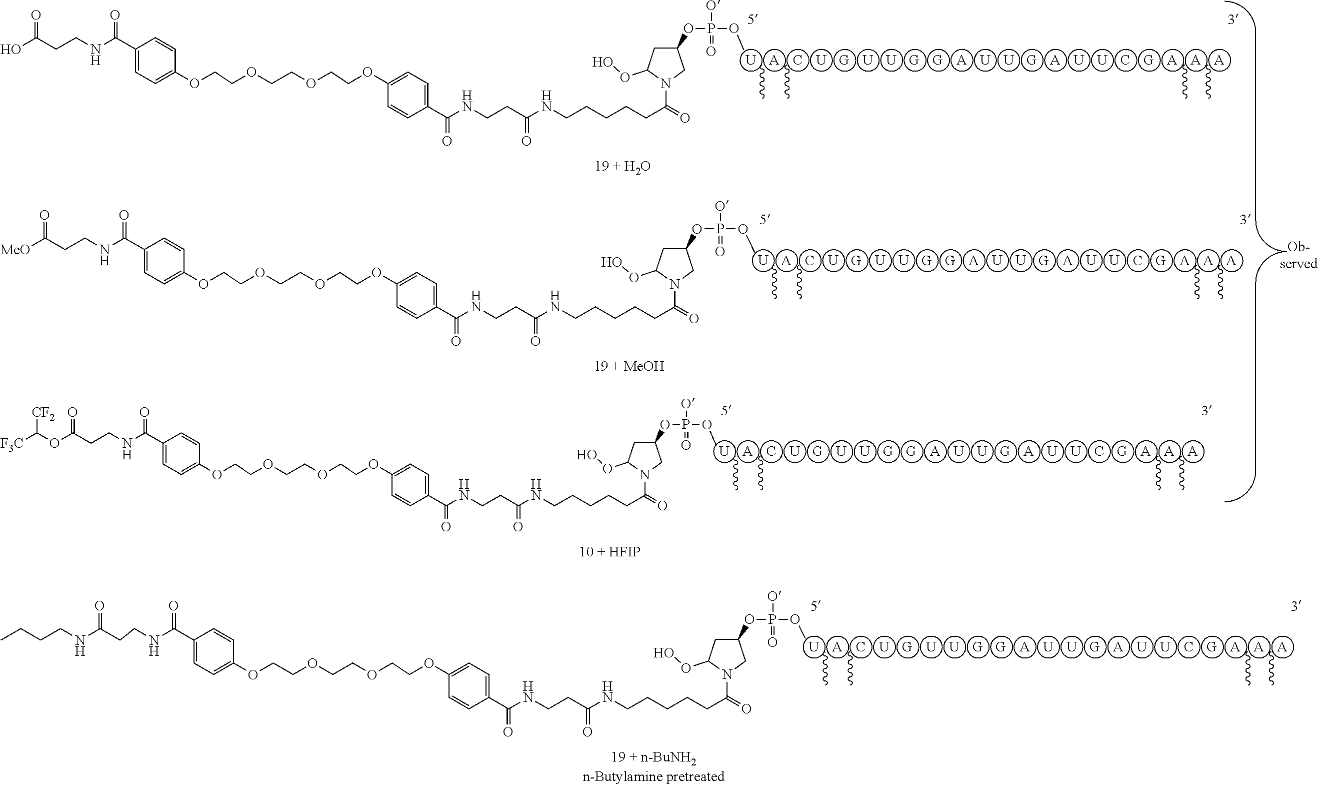

Double-stranded RNAs (duplexes) comprising β-lactam-conjugated strands: The β-lactam conjugated strands were annealed with complementary strands using a low temperature duplex annealing. Generally, the duplexes were annealed by lyophilization as follows.

The purified β-lactam sense strand as an aqueous solution was analyzed by UV spectrophotometry to obtain an exact concentration. A subsequent equimolar amount of antisense strand was added at a concentration of 20-100 mg/mL in water. The combined strands were vortexed for 30 seconds and centrifuged to the bottom of a conical tube. The strands were frozen on dry ice and lyophilized to a powder during which the two strands anneal to each other.

### REFERENCES

(1) Setten, R. L.; Rossi, J. J.; Han, S.-p. The current state and future directions of RNAi-based therapeutics. Nature Reviews Drug Discovery 2019.
(2) Semple, S. C.; Akinc, A.; Chen, J.; Sandhu, A. P.; Mui, B. L.; Cho, C. K.; Sah, D. W.; Stebbing, D.; Crosley, E. J.; Yaworski, E.; Hafez, I M.; Dorkin, J. R.; Qin, J.; Lam, K.; Rajeev, K. G.; Wong, K. F.; Jeffs, L. B.; Nechev, L.; Eisenhardt, M. L.; Jayaraman, M.; Kazem, M.; Maier, M. A.; Srinivasulu, M.; Weinstein, M. J.; Chen, Q.; Alvarez, R.; Barros, S. A.; De, S.; Klimuk, S. K.; Borland, T.; Kosovrasti, V.; Cantley, W. L.; Tam, Y. K.; Manoharan, M.; Ciufolini, M. A.; Tracy, M. A.; de Fougerolles, A.; MacLachlan, I.; Cullis, P. R.; Madden, T. D.; Hope, M. J. Rational design of cationic lipids for siRNA delivery. Nature biotechnology 2010, 28, 172-176.
(3) Jayaraman, M.; Ansell, S. M.; Mui, B. L.; Tam, Y. K.; Chen, J.; Du, X.; Butler, D.; Eltepu, L.; Matsuda, S.; Narayanannair, J. K.; Rajeev, K. G.; Hafez, I M.; Akinc, A.; Maier, M. A.; Tracy, M. A.; Cullis, P. R.; Madden, T. D.; Manoharan, M.; Hope, M. J. Maximizing the potency of siRNA lipid nanoparticles for hepatic gene silencing in vivo. Angewandte Chemie (International ed in English) 2012, 51, 8529-8533.
(4) Nair, J. K.; Willoughby, J. L.; Chan, A.; Charisse, K.; Alam, M. R.; Wang, Q.; Hoekstra, M.; Kandasamy, P.; Kel'in, A. V.; Milstein, S.; Taneja, N.; O'Shea, J.; Shaikh, S.; Zhang, L.; van der Sluis, R. J.; Jung, M. E.; Akinc, A.; Hutabarat, R.; Kuchimanchi, S.; Fitzgerald, K.; Zimmermann, T.; van Berkel, T. J.; Maier, M. A.; Rajeev, K. G.; Manoharan, M. Multivalent N-acetylgalactosamine-conjugated siRNA localizes in hepatocytes and elicits robust RNAi-mediated gene silencing. Journal of the American Chemical Society 2014, 136, 16958-16961.
(5) Weiner, G. J. Building better monoclonal antibody-based therapeutics. Nature reviews. Cancer 2015, 15, 361-370.
(6) Carter, P. J.; Lazar, G. A. Next generation antibody drugs: pursuit of the &#39; high-hanging fruit&#39. Nature Reviews Drug Discovery 2017, 17, 197.
(7) Beck, A.; Goetsch, L.; Dumontet, C.; Corvaia, N. Strategies and challenges for the next generation of antibody-drug conjugates. Nature reviews. Drug discovery 2017, 16, 315-337.
(8) Kumar, P.; Ban, H. S.; Kim, S. S.; Wu, H.; Pearson, T.; Greiner, D. L.; Laouar, A.; Yao, J.; Haridas, V.; Habiro, K.; Yang, Y. G.; Jeong, J. H.; Lee, K. Y.; Kim, Y. H.; Kim, S. W.; Peipp, M.; Fey, G. H.; Manjunath, N.; Shultz, L. D.; Lee, S. K.; Shankar, P. T cell-specific siRNA delivery suppresses HV-1 infection in humanized mice. Cell 2008, 134, 577-586.
(9) Song, E.; Zhu, P.; Lee, S. K.; Chowdhury, D.; Kussman, S.; Dykxhoom, D. M.; Feng, Y.; Palliser, D.; Weiner, D. B.; Shankar, P.; Marasco, W. A.; Lieberman, J. Antibody mediated in vivo delivery of small interfering RNAs via cell-surface receptors. Nature biotechnology 2005, 23, 709-717.
(10) Peer, D.; Zhu, P.; Carman, C. V.; Lieberman, J.; Shimaoka, M. Selective gene silencing in activated leukocytes by targeting siRNAs to the integrin lymphocyte function-associated antigen-1. Proceedings of the National Academy of Sciences of the United States of America 2007, 104, 4095-4100.
(11) Xia, C.-F.; Boado, R. J.; Pardridge, W. M. Antibody-Mediated Targeting of siRNA via the Human Insulin Receptor Using Avidin-Biotin Technology. Molecular Pharmaceutics 2009, 6, 747-751.
(12) Lu, H.; Wang, D.; Kazane, S.; Javahishvili, T.; Tian, F.; Song, F.; Sellers, A.; Barnett, B.; Schultz, P. G. Site-Specific Antibody-Polymer Conjugates for siRNA Delivery. Journal of the American Chemical Society 2013, 135, 13885-13891.
(13) Baumer, S.; Baumer, N.; Appel, N.; Terheyden, L.; Fremerey, J.; Schelhaas, S.; Wardelmann, E.; Buchholz, F.; Berdel, W. E.; Muller-Tidow, C. Antibody-mediated delivery of anti-KRAS-siRNA in vivo overcomes therapy resistance in colon cancer. Clinical cancer research: an official journal of the American Association for Cancer Research 2015, 21, 1383-1394.
(14) Baumer, N.; Appel, N.; Terheyden, L.; Buchholz, F.; Rossig, C.; Muller-Tidow, C.; Berdel, W. E.; Baumer, S. Antibody-coupled siRNA as an efficient method for in vivo mRNA knockdown. Nature protocols 2016, 11, 22-36.
(15) Sugo, T.; Terada, M.; Oikawa, T.; Miyata, K.; Nishimura, S.; Kenjo, E.; Ogasawara-Shimizu, M.; Makita, Y.; Imaichi, S.; Murata, S.; Otake, K.; Kikuchi, K.; Teratani, M.; Masuda, Y.; Kamei, T.; Takagahara, S.; Ikeda, S.; Ohtaki, T.; Matsumoto, H. Development of antibody-siRNA conjugate targeted to cardiac and skeletal muscles. Journal of controlled release:official journal of the Controlled Release Society 2016, 237, 1-13.
(16) Lu, H.; Wang, D.; Kazane, S.; Javahishvili, T.; Tian, F.; Song, F.; Sellers, A.; Barnett, B.; Schultz, P. G. Site-specific antibody-polymer conjugates for siRNA delivery. J Am Chem Soc 2013, 135, 13885-13891.
(17) Cuellar, T. L.; Barnes, D.; Nelson, C.; Tanguay, J.; Yu, S. F.; Wen, X.; Scales, S. J.; Gesch, J.; Davis, D.; van Brabant Smith, A.; Leake, D.; Vandlen, R.; Siebel, C. W. Systematic evaluation of antibody-mediated siRNA delivery using an industrial platform of THIOMAB-siRNA conjugates. Nucleic acids research 2015, 43, 1189-1203.
(18) Nanna, A. R.; Li, X.; Walseng, E.; Pedzisa, L.; Goydel, R. S.; Hymel, D.; Burke Jr, T. R.; Roush, W. R.; Rader, C. Harnessing a catalytic lysine residue for the one-step preparation of homogeneous antibody-drug conjugates. Nature Communications 2017, 8, 1112.
(19) Kyle, R. A.; Rajkumar, S. V. Multiple myeloma. The New England journal of medicine 2004, 351, 1860-1873.
(20) Ikeda, H.; Hideshima, T.; Fulciniti, M.; Lutz, R. J.; Yasui, H.; Okawa, Y.; Kiziltepe, T.; Vallet, S.; Pozzi, S.; Santo, L.; Perrone, G.; Tai, Y. T.; Cirstea, D.; Raje, N. S.; Uherek, C.; Dalken, B.; Aigner, S.; Osterroth, F.; Munshi, N.; Richardson, P.; Anderson, K. C. The monoclonal antibody nBT062 conjugated to cytotoxic Maytansinoids has selective cytotoxicity against CD138-positive multiple myeloma cells in vitro and in vivo. Clinical cancer research:an official journal of the American Association for Cancer Research 2009, 15, 4028-4037.
(21) Tai, Y. T.; Mayes, P. A.; Acharya, C.; Zhong, M. Y.; Cea, M.; Cagnetta, A.; Craigen, J.; Yates, J.; Gliddon, L.; Fieles, W.; Hoang, B.; Tunstead, J.; Christie, A. L.; Kung, A. L.; Richardson, P.; Munshi, N. C.; Anderson, K. C. Novel anti-B-cell maturation antigen antibody-drug conjugate (GSK2857916) selectively induces killing of multiple myeloma. Blood 2014, 123, 3128-3138.
(22) Trudel, S.; Lendvai, N.; Popat, R.; Voorhees, P. M.; Reeves, B.; Libby, E. N.; Richardson, P. G.; Anderson, L. D., Jr.; Sutherland, H. J.; Yong, K.; Hoos, A.; Gorczyca, M. M.; Lahiri, S.; He, Z.; Austin, D. J.; Opalinska, J. B.; Cohen, A. D. Targeting B-cell maturation antigen with GSK2857916 antibody-drug conjugate in relapsed or refractory multiple myeloma (BMA117159): a dose escalation and expansion phase 1 trial. The Lancet. Oncology 2018, 19, 1641-1653.
(23) Trudel, S.; Lendvai, N.; Popat, R.; Voorhees, P. M.; Reeves, B.; Libby, E. N.; Richardson, P. G.; Hoos, A.; Gupta, I.; Bragulat, V.; He, Z.; Opalinska, J. B.; Cohen, A. D. Antibody-drug conjugate, GSK2857916, in relapsed/refractory multiple myeloma: an update on safety and efficacy from dose expansion phase I study. Blood cancer journal 2019, 9, 37.
(24) Kumar, S. K.; Anderson, K. C. Immune Therapies in Multiple Myeloma. Clinical cancer research:an official journal of the American Association for Cancer Research 2016, 22, 5453-5460.
(25) Schmeel, L. C.; Schmeel, F. C.; Kim, Y.; Endo, T.; Lu, D.; Schmidt-Wolf, I. G. Targeting the Wnt/beta-catenin pathway in multiple myeloma. Anticancer research 2013, 33, 4719-4726.
(26) Su, N.; Wang, P.; Li, Y. Role of Wnt/β-catenin pathway in inducing autophagy and apoptosis in multiple myeloma cells. Oncology letters 2016, 12, 4623-4629.
(27) List, B.; Barbas, C. F., 3rd; Lerner, R. A. Aldol sensors for the rapid generation of tunable fluorescence by antibody catalysis. Proceedings of the National Academy of Sciences of the United States of America 1998, 95, 15351-15355.
(28) Gu, J.; Yang, J.; Chang, Q.; Lu, X.; Wang, J.; Chen, M.; Ghayur, T.; Gu, J. Identification of anti-ErbB2 dual variable domain immunoglobulin (DVD-Ig) proteins with unique activities. PloS one 2014, 9, e97292.
(29) Gardner, M. R.; Fellinger, C. H.; Prasad, N. R.; Zhou, A. S.; Kondur, H. R.; Joshi, V. R.; Quinlan, B. D.; Farzan, M. CD4-Induced Antibodies Promote Association of the HIV-1 Envelope Glycoprotein with CD4-Binding Site Antibodies. Journal of Virology 2016, 90, 7822-7832.
(30) Sinha, S. C.; Das, S.; Li, L. S.; Lerner, R. A.; Barbas, C. F., 3rd Preparation of integrin alpha(v)beta3-targeting Ab 38C2 constructs. Nature protocols 2007, 2, 449-456.
(31) Livak, K. J.; Schmittgen, T. D. Analysis of relative gene expression data using real-time quantitative PCR and the 2(-Delta Delta C(T)) Method Methods (San Diego, Calif.) 2001, 25, 402-408.
(32) Lv, J.; Cao, X. F.; Ji, L.; Zhu, B.; Wang, D. D.; Tao, L.; Li, S. Q. Association of beta-catenin, Wnt1, Smad4, Hoxa9, and Bmi-1 with the prognosis of esophageal squamous cell carcinoma. Medical oncology (Northwood, London, England) 2012, 29, 151-160.
(33) Tsang, W. Y.; Ahmed, N.; Hinchliffe, P. S.; Wood, J. M.; Harding, L. P.; Laws, A. P.; Page, M. I. Different transition-state structures for the reactions of beta-lactams and analogous beta-sultams with serine beta-lactamases. Journal of the American Chemical Society 2005, 127, 17556-17564.
(34) Parmar, R. G.; Brown, C. R.; Matsuda, S.; Willoughby, J. L. S.; Theile, C. S.; Charissé, K.; Foster, D. J.; Zlatev, I.; Jadhav, V.; Maier, M. A.; Egli, M.; Manoharan, M.; Rajeev, K. G. Facile Synthesis, Geometry, and 2′-Substituent-Dependent in Vivo Activity of 5′-(E)- and 5′-(Z)-Vinylphosphonate-Modified siRNA Conjugates. Journal of Medicinal Chemistry 2018, 61, 734-744.

### Example 2

Cell lines: Human multiple myeloma (MM) cell lines NCI-H929 and MM.1S were purchased directly from American Type Culture Collection (ATCC), and subsequently cultured in RPMI-1640 medium that was supplemented with 10% (v/v) FBS, 100 μg/mL streptomycin, and 100 U/mL penicillin (Thermo Fisher Scientific) at 37° C., 5% CO₂ and 100% humidity. In addition, Expi293F cells for antibody production were grown in Expi293 Expression Medium (Thermo Fisher Scientific) at 37° C., 8% CO₂ and 100% humidity.

BCMA Antigen expression: The BCMA sequence was obtained from NCBI GenBank (accession #NM_001192). Subsequently, Fc-BCMA was cloned, expressed, followed by purification and biotinylation as described in previous literature (Peng et al., 2017).

Library Selection: Anti-BCMA antibodies were selected via phage display from a previously described (Peng et al., 2017) naïve chimeric rabbit/human library against biotinylated Fc-BCMA with a total of four rounds of panning. Briefly, streptavidin-coated magnetic beads (Dynabeads MyOne Streptavidin C1, Thermo Fisher) were incubated with the biotinylated antigen, and subsequently employed to competitively select anti-BCMA antibodies from the phage library as described previously (Peng et al., 2017). Polyclonal human IgG (Pierce) was added during each round of panning as decoy at a concentration of 5 μg/μL. Multiple clones tested positive by ELISA against Fc-BCMA after selection. Upon further analysis via DNA fingerprinting and sequencing, these clones revealed only one unique kappa clone (VOO1) against Fc-BCMA.

Humanization of VOO1: Briefly, an online surveillance was employed to search out closely similar human germline(s) using the IgBlast website with the least amount of polymorphisms. Subsequently, the heavy chain was designed using IMGT's IGHV database while the light chain was designed using IGKV database. All six complementarity-determining regions (CDRs) of VOO1 were grafted into human germline framework regions (FRs). Various mutations to the FRs were conducted by gradually replacing human germline to VOO1 rabbit FR residues without severely affecting the binding affinity. A similar humanization protocol for a rabbit antibody was previously published (Goydel et al., 2020).

Fab cloning, expression and purification: The selected chimeric rabbit/human clone VOO1 (in bacterial vector pC3C) or humanized VOO1 (hVOO1) Fab was cloned into pET11a using the restriction enzyme Sfil, and transformed into E. coli strain Rosetta (DE3) (EMD Millipore) and cultured in autoinduction media. The Fab protein was purified from the supernatant with CaptureSelect CH1-XL pre-packed column, and purity was analyzed via SDS-PAGE and Coomassie staining of both reduced and non-reduced Fabs. Yields generated were in the range of 1-5 mg/L of culture media. Amino acid sequences of VOO1 and hVOO1 Fab are displayed in the order variable light chain (VL) followed by variable heavy chain (VH) domain.

VOO1 Fab VL:
(SEQ ID NO: 11)
DVVMTQTPSSVPAAVGGTVTINCQASQSIDSNLAWFQQKPGQPPNLLIY
DASTLASGVPSRFKGSAGKQFTLTISGVQREDAATYYCLGSYSRTEKAF
GAGTKVEIK
VOO1 Fab VH:
(SEQ ID NO: 12)
QEQLEESGGRLVTPGTPLTLTCTVSGFSLSNYHMSWVRQAPGKGLEWIG
FITSGGSTYYASWAKGRFTISRTSTTVDLKITSPTTEDTATYFCARWNG
YGGNMWGPGTLVTVSS
hVOO1 Fab VL:
(SEQ ID NO: 13)
DIQMTQSPSSLSASVGDRVTITCQASQSIDSNLAWYQQKPGKVPKLLIY
DASTLASGVPSRFSGSGSGTDFTLTISSLQPEDVATYYCLGSYSRTEKA
FGGGTKVEIK
hVOO1 Fab VH:
(SEQ ID NO: 14)
EVQLVESGGGLVQPGGSLRLSCAASGFTLSNYHMSWVRQAPGKGLEWVS
FITSGGSTYYASWAKGRFTISRDNSKNTLYLQMNSLRAEDTAVYYCARW
NGYGGNMWGQGTLVTVS

Cloning, expression and purification of IgG1- and IgM-based VOO1 and hVOO1: The heterodimeric (knobs-into-holes) VOO1×(h38C2)₂ IgG1 was cloned and expressed as previously described (Qi et al., 2019) by replacing v9 scFv with VOO1 scFv (VL-(G₄S)₃-VH). As for VOO1/hVOO1×h38C2 DVD-IgMs, the variable domain encoding cDNA sequence of h38C2 was PCR-amplified from VOO1×(h38C2)₂ and the constant domains (Cμ2-Cμ3-Cμ4) of the heavy chain of human IgM were cloned from a previously published plasmid (Vire et al., 2014). The h38C2 IgM (light and heavy chains) was assembled by overlap extension PCR and NheI/BamHI-cloned into previously described mammalian expression vector (Gardner et al., 2016). All plasmids were transiently transfected into Expi293F cells using ExpiFectamine according to the manufacturer's instructions (Thermo Fisher Scientific). After 5 days, the media were collected, followed by addition of ammonium sulphate (0.8 M) and sodium phosphate (20 mM final concentration), and finally filtered through a 0.22-μm filter. The purification was performed using a 1-ml HiTrap IgM Purification HP column (GE Healthcare) together with an ÄKTA FPLC instrument (GE Healthcare), and the IgM was finally eluted off the column with 20 mM sodium phosphate. Yields were typically in the range of 20-40 mg/L. The purity of VOO1×(h38C2)₂ IgG1 and VOO1/hVOO1×h38C2 DVD-IgMs was confirmed by nonreducing and reducing SDS-PAGE followed by Coomassie blue staining, and the concentration was determined by measuring the absorbance at 280 nm and BCA assay. Amino acid sequences of DVD IgG1s in the order signal sequence—outer variable domain—spacer—inner variable domain—constant domain(s):

VOO1x(h38C2)₂ IgG1
(1) VOO1 VL-(G₄S)₃-VH-hinge-CH2-CH3 (holes):
(SEQ ID NO: 28)
MDWTWRILFLVAAATGAHSDVVMTQTPSSVPAAVGGTVTINCQASQSIDSNLA
WFQQKPGQPPNLLIYDASTLASGVPSRFKGSGAGKQFTLTISGVQREDAATYYCL
GSYSRTEKAFGAGTKVEIKGGGGSGGGGSGGGGSQEQLEESGGRLVTPGTPLTLT
CTVSGFSLSNYHMSWVRQAPGKGLEWIGFITSGGSTYYASWAKGRFTISRTSTTV
DLKITSPTTEDTATYFCARWNGYGGNMWGPGTLVTVEPKSSDKTHTCPPCPAPE
LLGGPSVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTISKAKG
QPREPQVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPGA
(2) (h38C2)₂ VH-CH1-EPKSCD(G₄S)2-VH-CH1-hinge-CH2-CH3 (knobs):
(SEQ ID NO: 29)
MDWTWRILFLVAAATGAHSEVQLVESGGGLVQPGGSLRLSCAASGFTFSNYWM
SWVRQSPEKGLEWVSEIRLRSDNYATHYAESVKGRFTISRDNSKNTLYLQMNSL
RAEDTGIYYCKTYFYSFSYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQT
YICNVNHKPSNTKVDKRVEPKSCDGGGGSGGGGSEVQLVESGGGLVQPGGSLRL
SCAASGFTFSNYWMSWVRQSPEKGLEWVSEIRLRSDNYATHYAESVKGRFTISR
DNSKNTLYLQMNSLRAEDTGIYYCKTYFYSFSYWGQGTLVTVSSASTKGPSVFP
LAPSSKSTSGGTAALGCLVKDYFPEPVTVSWNSGALTSGVHTFPAVLQSSGLYSL
SSWTVPSSSLGTQTYICNVNHKPSNTKVDKRVEPKSSDKTHTCPPCPAPELLGGP
SVFLFPPKPKDTLMISRTPEVTCVVVDVSHEDPEVKFNWYVDGVEVHNAKTKPR
EEQYASTYRVVSVLTVLHQDWLNGKEYKCKVSNKALPAPIEKTISKAKGQPREP
QVYTLPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGA
(3) h38C2VL-CL:
(SEQ ID NO: 30)
MDWTWRILFLVAAATGAHSELQMTQSPSSLSASVGDRVTITCRSSQSLLHTYGSP
YLNWYLQKPGQSPKLLIYKVSNRFSGVPSRFSGSGSGTDFTLTISSLQPEDFAVYF
CSQGTHLPYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEV
THQGLSSPVTKSFNRGEC

Cloning, expression, and purification of anti-SLAMF7 IgG1-EEP: This construct was based on humanized anti-human SLAMF7 (also known as CS-1) mAb hERCS-409 disclosed in PCT/EP2018/071429 and the previously published endosomal escape peptide (EEP) aurein 1.2 (Li et al., 2015) in reverse amino acid sequence at the C-terminus of the light chain. Both light and heavy chains of anti-SLAMF7 IgG1-EEP were assembled by overlap extension PCR and NheI/BamHI-cloned into a previously published mammalian expression vector (Gardner et al., 2016).

hERCS-409-h38C2 DVD-IgG1-EEP:
(1) Light Chain:
(SEQ ID NO: 26)
MPMGSLQPLATLYLLGMLVADQQLTQSPSSLSASVGDRVTITCRAS
QSIGSWLSWYQQKPGKAPKLLIYGASNLASGVPSRFSGSRSGTDYT
LTISSLQPEDFATYYCLGASPNGWAFGQGTKVEIKASTKGPELQMT
QSPSSLSASVGDRVTITCRSSQSLLHTYGSPYLNWYLQKPGQSPKL
LIYKVSNRFSGVPSRFSGSGSGTDFTLTISSLQPEDFAVYFCSQGT
HLPYTFGGGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGECFSEAIKKIIDFLG
(2) Heavy Chain:
(SEQ ID NO: 27)
MPMGSLQPLATLYLLGMLVAEQQVVESGGGLVQPGGSLRLSCAVSG
FSLNSYGVIWVRQAPGKGLEYVSIIGSSGNTYYASSVKGRFTISRD
TRLNTVYLQMNSLRAEDTAVYFCARYYGDSGFDSWGQGTLVTVSSA
STKGPEVQLVESGGGLVQPGGSLRLSCAASGFTFSNYWMSWVRQSP
EKGLEWVSEIRLRSDNYATHYAESVKGRFTISRDNSKNTLYLQMNS
LRAEDTGIYYCKTYFYSFSYWGQGTLVTVSSASTKGPSVFPLAPSS
KSTSGGTAALGCLVKDYFPEPVTVSWNSGALTSGVHTFPAVLQSSG
LYSLSSVVTVPSSSLGTQTYICNVNHKPSNTKVDKRVEPKSCDKTH
TCPPCPAPELLGGPSVFLFPPKPKDTLMISRTPEVTCVVVDVSHED
PEVKFNWYVDGVEVHNAKTKPREEQYNSTYRVVSVLTVLHQDWLNG
KEYKCKVSNKALPAPIEKTISKAKGQPREPQVYTLPPSREEMTKNQ
VSLTCLVKGFYPSDIAVEWESNGQPENNYKTTPPVLDSDGSFFLYS
KLTVDKSRWQQGNVFSCSVMHEALHNHYTQKSLSLSPGA

Antibody conjugation: Conjugations were performed in PBS (pH 7.4) after the various IgMs constructs were diluted to 10 mg/mL (10.5 μM). In general, 10 μL of 3 mM beta lactam linker functionalized siRNA in H₂O (13.5 eq) was added to 2 mg of each IgM construct. The mixture was incubated overnight at 4° C. As for VOO1×(h38C2)₂ IgG bio-conjugation, 5 equivalents (eq) of lactam linker-functionalized siRNA was added using 5 μL of 3 mM siRNA in H₂O and incubated with 1 mg of IgG (11.4 μM) for 4 h at RT. Complete conjugation was analyzed using a methodol assay as described previously (Nanna et al., 2017). Upon completion, unreacted compound was removed by using size exclusion chromatography (SEC) as described before (Nanna et al., 2020). The antibody-siRNA conjugates (ARCs) obtained after SEC were then concentrated using a 15-mL Millipore Ultra-15 30-kDa cut-off Centrifugal Filter Unit, washed with 4 mL of PBS three times, and were concentrated to a final volume of about 250 μL. The concentration of the ARCs was measured with a BCA assay, using BSA as standard. The samples were loaded on a NuPAGE Novex Bis-Tris 4-12% gradient gel in SDS-PAGE Sample Buffer, and the gel was stained with Coomassie blue followed by washing to determine the purity.

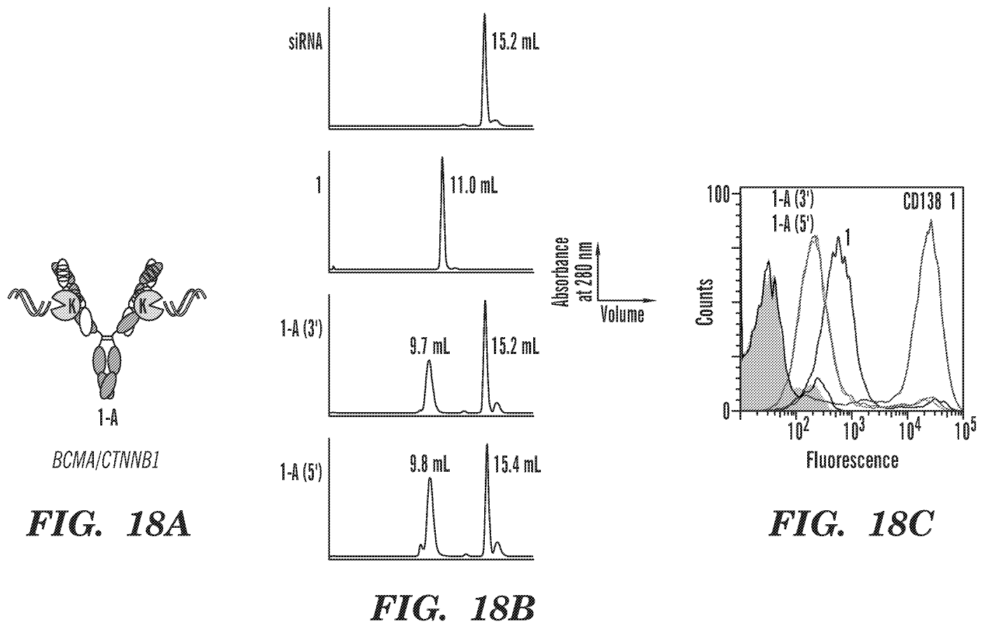

Size-exclusion chromatography (SEC): The ÄKTA FPLC instrument was utilized to perform SEC by installing a Superdex® 200 10/300 GL column (GE Healthcare) for separation. During analytical runs, 15 μg (not exceeding 200 μL) of sample was loaded into the loop and analyzed under a flow rate of 0.5 mL/min in PBS and at a wavelength of 280 nm. As for actual ARC purification, 1-2 mg of ARC (not exceeding 700 μL) was loaded and passed through the SEC column, with the desired ARC peak separated from the free siRNA that was not conjugated. Exemplary results are shown in FIG. 18.

Catalytic activity assay: The methodol assay was employed to detect the catalytic activity of unreacted reactive lysine within the h38C2 fragment within the antibody after conjugation as described previously (Nanna et al., 2017; Nanna and Rader, 2019). Both antibodies and ARCs were made up to a concentration of 1 μM in PBS (pH 7.4) and a volume of 98 μL, before aliquoting them into a 96-well plate with triplicates of each sample. Subsequently, 2 μL of 10 mM methodol in ethanol was added and the fluorescence was determined using a SpectraMax M5 instrument (Molecular Devices) with SoftMax Pro software. The excitation wavelength (ext) was set to 330 nm, while the emission wavelength (em) was calibrated to 452 nm, starting at time point of 0 min while obtaining data at 5-min intervals. Finally, all data were normalized to the data set of 98 μL PBS with 2 μL of the methodol solution added.

Flow cytometry: During the process of flow cytometry, 100,000 cells were dispensed in each well of a V-bottom 96-well plate (Corning) and washed with 150 μL of flow cytometry buffer (PBS, 2% (v/v) FBS, 0.01% (w/v) NaN₃, pH 7.4). Subsequently, the cells were incubated with the antibodies (200 nM in 50 μL PBS) for 1 h on ice, followed by 3 rounds of washing (200 μL flow buffer). Finally, the cells were stained with R-Phycoerythrin AffiniPure F(ab′)₂ Fragment Goat Anti-Human IgG, F(ab′)₂ fragment specific (for Fabs and IgG1 or IgG1 ARCs), or R-Phycoerythrin AffiniPure F(ab′)₂ Fragment Donkey Anti-Human IgM, Fc_{5μ} fragment specific (IgM or IgM ARCs) purchased from Jackson ImmunoResearch Laboratories for 30 min on ice. After 3 rounds of washing, the cells were passed through a BD FACSCanto™ II and the data were extracted and analyzed via FlowJo software. Exemplary results are shown in FIG. 18.

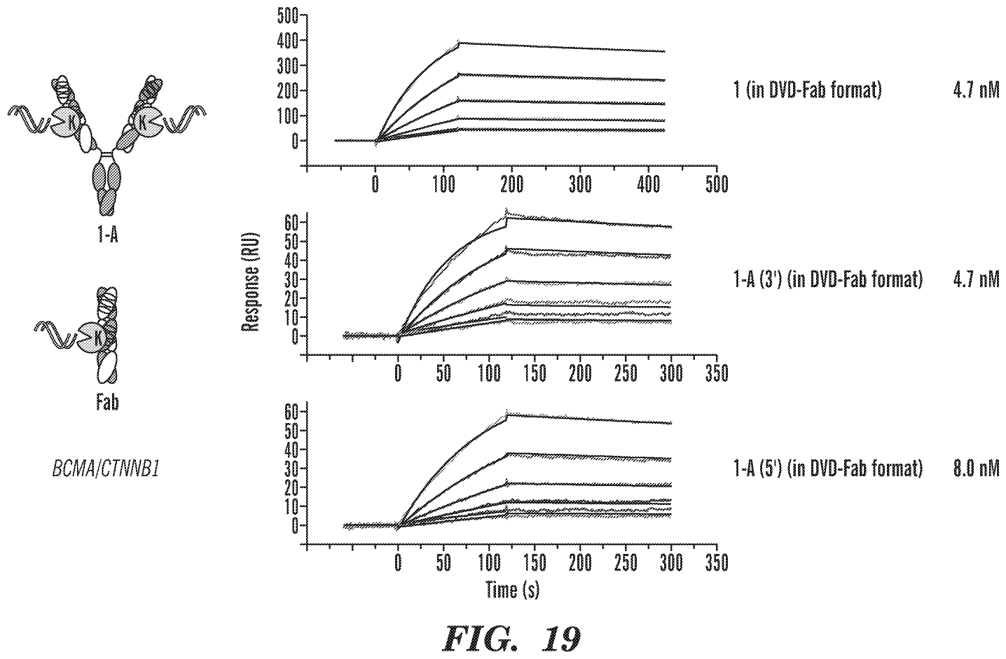

Surface plasmon resonance (SPR): SPR was used to determine the kinetic and thermodynamic BCMA binding parameters of the anti-BCMA Fabs VOO1 and hVOO1. A Biacore X100 instrument was used with Biacore reagents and software (GE Healthcare). A mouse anti-human IgG (CH₂ domain) mAb was immobilized on a CM5 sensor chip using reagents and instructions supplied with the Human Antibody Capture Kit (GE Healthcare). A human BCMA-Fc fusion protein (R&D Systems) was captured at a density not exceeding 1,000 RU. Each sensor chip included an empty flow cell for instantaneous background depletion. All binding assays used 1×HBSEP+ running buffer (10 mM HEPES, 150 mM NaCl, 3 mM EDTA (pH 7.4) and 0.05% (v/v) Surfactant P20) and a flow rate of 30 μL/min. For affinity measurements, VOO1 and hVOO1 Fabs were injected at five different concentrations 12.5 (twice), 25, 50, 100 and 200 nM). The sensor chips were regenerated with 3 M MgCl₂ from the Human Antibody Capture Kit without any loss of binding capacity. Calculation of association (kₒₙ) and dissociation (k_{off}) rate constants was based on a 1:1 Langmuir binding model. The equilibrium dissociation constant (K_{d}) was calculated from k_{off}/kₒₙ. Exemplary results are shown in FIG. 19.

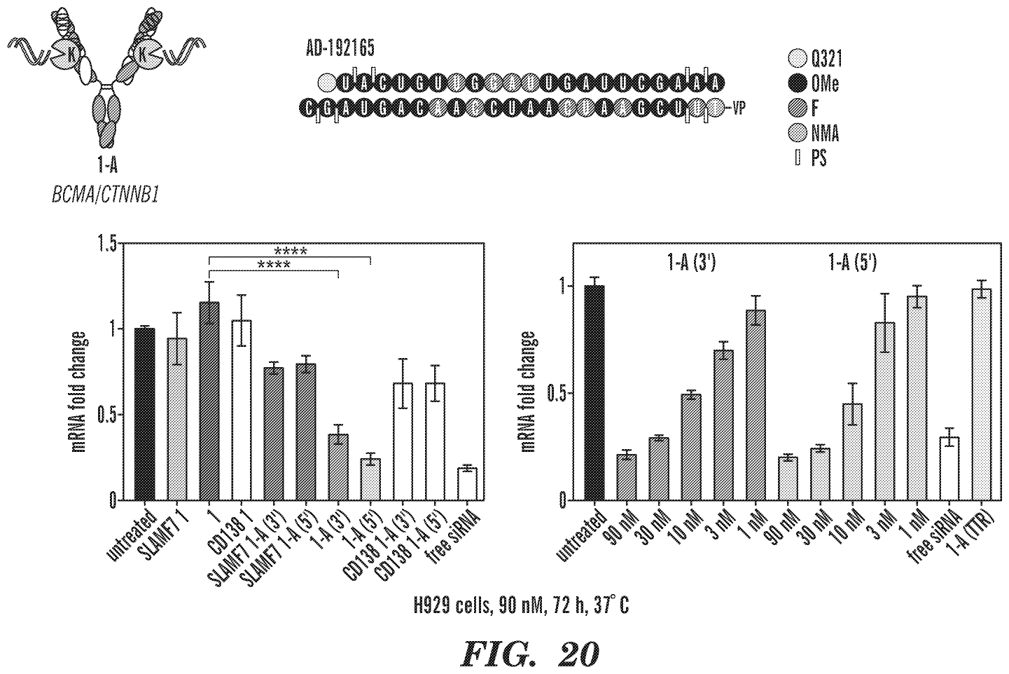

RT-qPCR: To analyze mRNA knockdown by RT-qPCR, 500 μL of cells (200,000 cells per well) were dispensed in a 12-well cell culture dish initially. Subsequently, 500 μL of VOO1×(h38C2)₂ IgG1, IgG1 ARCs, VOO1/hVOO1×h38C2 IgM, and its corresponding ARCs were diluted with RPMI 1640 medium (10% (v/v) FBS supplemented with 100 μg/ml streptomycin and 100 U/ml penicillin) was immediately added to the desired concentrations. Free IRF4 siRNA was diluted to 18 μM in 50 μL of Opti-MEM medium, and subsequently added to a mixture of 3 μL of Lipofectamine RNAiMAX Transfection Reagent (Thermo Fisher Scientific) in 47 μL of Opti-MEM following the manufacturer's instructions. After 5-min incubation at RT, 100 μL of this mixture was added to cells (200,000 cells per well), and finally made up to 1 mL with RPMI 1640 medium (final siRNA concentration of 1.8 μM). After 48 h, extraction of RNA from cell lysates was performed using the RNeasy Mini Kit (Qiagen) followed by cDNA synthesis of 1 μg DNase-digested RNA, using the Maxima First-Strand cDNA Synthesis Kit for RT-qPCR (Invitrogen) according to the manufacturer's instructions. qPCR of the synthesized cDNA was performed using the SYBR Green PCR Master Mix (Thermo Fisher Scientific) according to the manufacturer's instructions, performed on an Applied Biosystems' StepOnePlus Real-Time PCR System, and analyzed using StepOne Software v2.2.2 (both from Thermo Fisher Scientific). All measurements were conducted three times using biological duplicates or triplicates and standardized to the levels of β-actin. Relative changes in gene expression were calculated according to the 2-CT algorithm (29). Primer sequences (30): IRF4 Forward: [5′-TCACCACTGCCAGCTGCTA-3′ (SEQ ID NO: 49)]; IRF4 Reverse: [5′-AAACTCCGGATGGCCTCAT-3′ (SEQ ID NO: 50)]; Actin Forward: [5′-CCTGTACGCCAACACAGTGC-3′ (SEQ ID NO: 42)]; Actin Reverse: [5′-ATACTCCTGCTTGCTGATCC-3′ (SEQ ID NO: 43)]. Exemplary results are shown in FIG. 20.

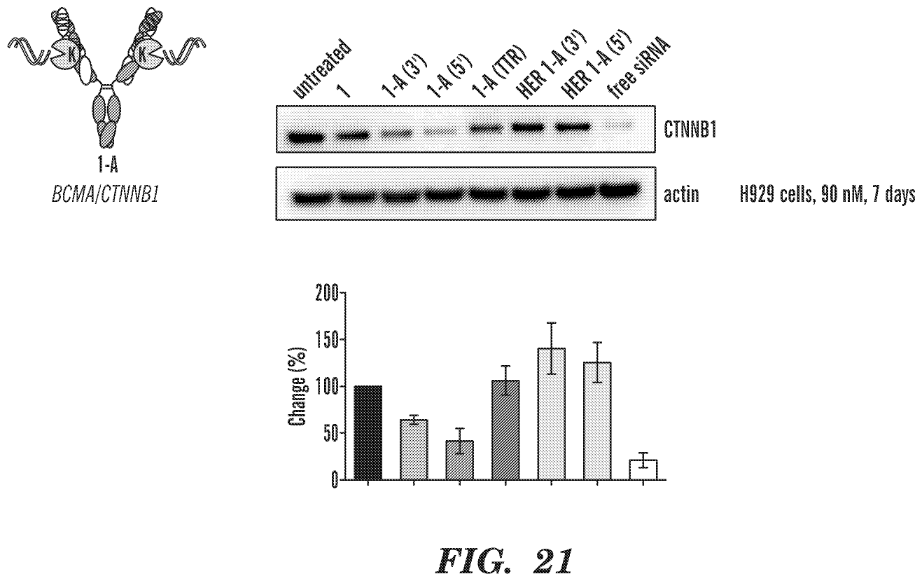

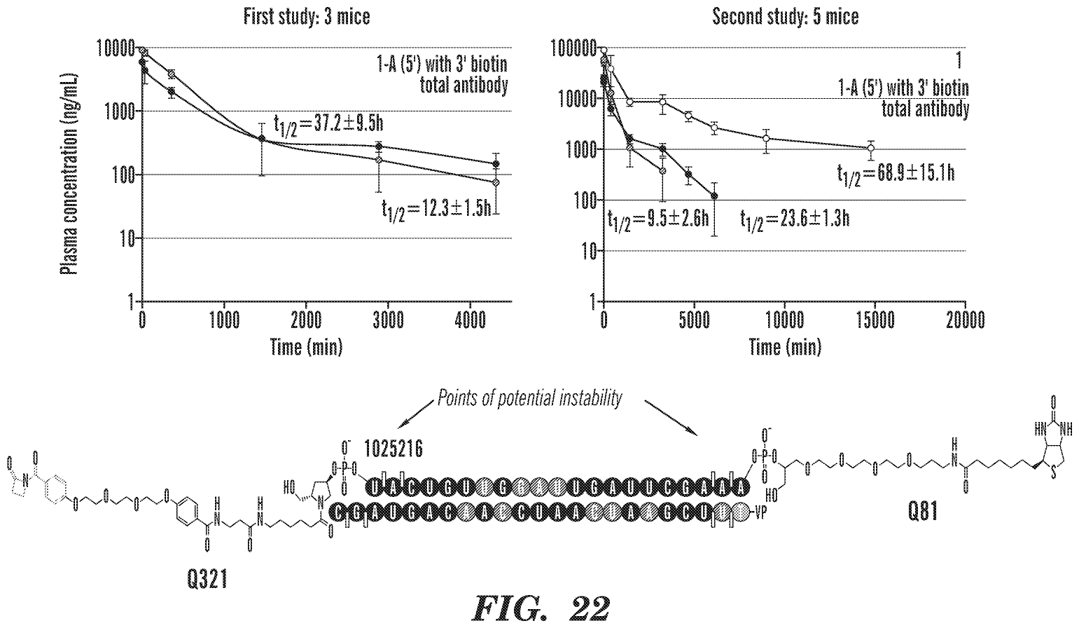

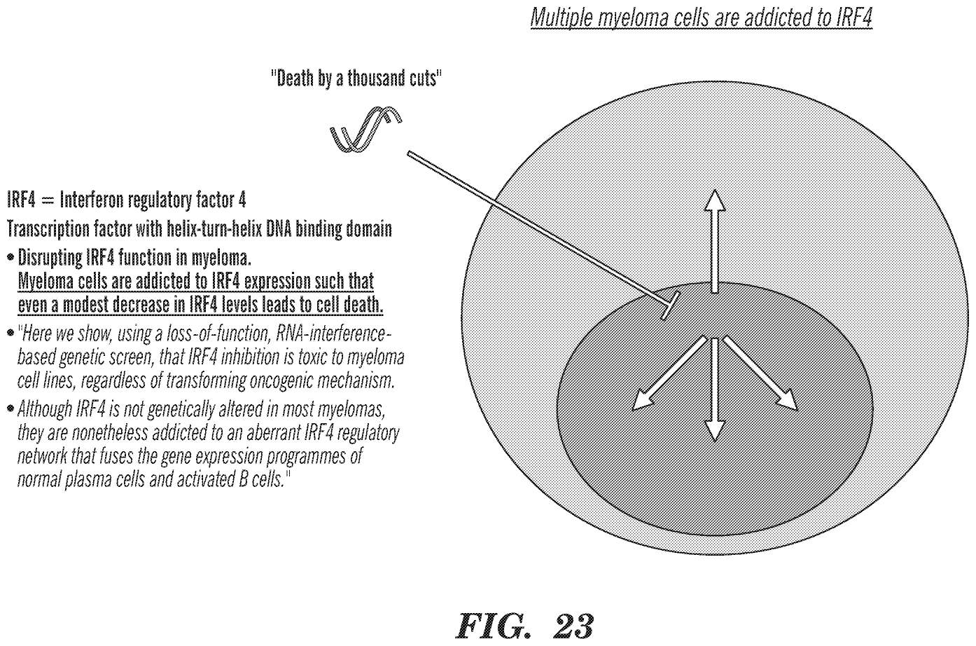

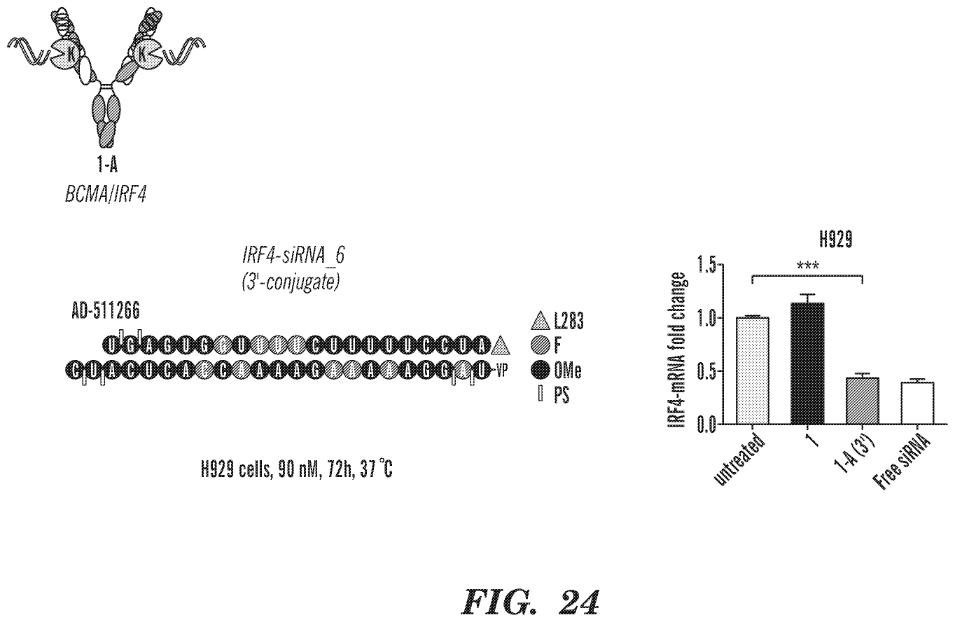

Western blotting: To analyze protein knockdown via western blotting, 500 μL of cells (200,000 cells per well) were dispensed in a 12-well cell culture dish initially. Subsequently, 500 μL of VOO1×(h38C2)₂ IgG1, IgG1 ARCs, VOO1/hVOO1×h38C2 IgM, and its corresponding ARCs were diluted with RPMI 1640 medium (10% (v/v) FBS supplemented with 100 μg/ml streptomycin and 100 U/ml penicillin) and immediately added to their desired concentrations. Free IRF4 siRNA was diluted to 18 μM in 50 μL of Opti-MEM medium, and subsequently added to a mixture of 3 μL of Lipofectamine RNAiMAX Transfection Reagent (Thermo Fisher Scientific) in 47 μL of Opti-MEM following the manufacturer's instructions. After 5 min incubation at RT, 100 μL of this mixture was added to the cells (200,000 cells per well), and finally made up to 1 mL with RPMI 1640 medium (final siRNA concentration of 1.8 μM). After 48 h, the cells were washed with PBS and lysed using RIPA Lysis Buffer (Thermo Fisher Scientific) containing a protease inhibitor cocktail and EDTA (Thermo Fisher Scientific). The samples were diluted with 1x NuPAGE LDS sample buffer (Thermo Fisher Scientific) containing 2% (v/v) β-mercaptoethanol and boiled at 100° C. for 5 min before running on NuPAGE Novex 4-12% Bis-Tris gels (Thermo Fisher Scientific). After transfer to a polyvinylidene difluoride (PVDF) membrane (Millipore) and blocking with 10% (v/v) Western Blocking Reagent (Thermo Fisher, cat. no. 37532) in Tris-buffered saline containing 0.01% Tween 20 (TBST), the PVDF membrane was incubated with 0.286 μg/mL rabbit anti-MUM1 (IRF4) mAb EP5699 (Abcam, cat. no. ab133590) in 10% (v/v) Western Blocking Reagent in TBST at 4° C. overnight. The PVDF membrane was washed with TBST followed by incubation with a 1:10,000 dilution (10% (v/v) Western Blocking Reagent in TBST) of Peroxidase AffiniPure Goat Anti-Rabbit IgG (H+L) polyclonal antibody (Jackson ImmunoResearch Laboratories, cat. no. 111-035-144) at RT for 1 h before washing with TBST and development using ECL Prime Western Blotting Detection Reagent (GE Healthcare). The membrane was stripped thereafter and re-stained for housekeeping protein R-actin after blocking. For actin staining, the membrane was incubated with a 1:10,000 dilution (10% (v/v) Western Blocking Reagent in TBST) of peroxidase-conjugated mouse anti-human R-actin mAb AC-15 (Sigma Aldrich, cat. no. A3854) at 4° C. overnight, washed with TBST, and then imaged using ECL Prime Western Blotting Detection Reagent. ImageJ software was used for quantification. Exemplary results are shown in FIG. 21.

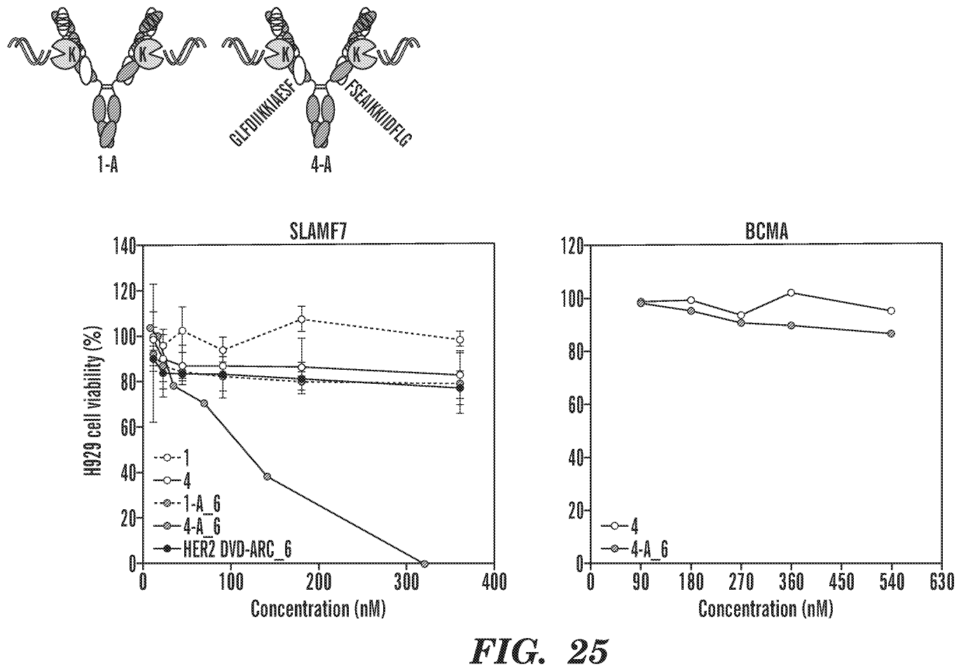

Cytotoxicity assay: To determine the cytotoxicity of the various antibody constructs and siRNA, NCI-H929 or MM.1S cells were plated in 96-well plates at 2×10⁴ cells per well initially in 50 μL. Subsequently, serial dilutions of unconjugated antibody and ARCs were added to the cells at concentrations ranging from 11.3 to 360 nM. Free IRF4 siRNA was diluted to 5.4 μM in 5 μL of Opti-MEM medium, and subsequently added to a mixture of 0.3 μL of Lipofectamine RNAiMAX Transfection Reagent (Thermo Fisher Scientific) in 4.7 μL of Opti-MEM following the manufacturer's instructions. After 5 min incubation at RT, 10 μL of this mixture was diluted to the desired concentration before addition to the cells (20,000 cells per well), and finally made up to 100 μL with RPMI 1640 medium. All samples were performed in triplicates. After incubation for 72 h, the cell viability was measured using the CellTiter 96 AQueous One Solution Cell Proliferation Assay (Promega) following the manufacturer's instructions. The cell viability was calculated as a percentage of untreated cells (≡100%), with cells treated with bortezomib (200 nM) used as positive control (≡0%). Exemplary results are shown in FIG. 25.

### REFERENCES

1. Gardner, M. R., Fellinger, C. H., Prasad, N. R., Zhou, A. S., Kondur, H. R., Joshi, V. R., Quinlan, B. D., and Farzan, M. (2016). CD4-induced antibodies promote association of the HIV-1 envelope glycoprotein with CD4-binding site antibodies. J Virol 90, 7822-7832.
2. Goydel, R. S., Weber, J., Peng, H., Qi, J., Soden, J., Freeth, J., Park, H., and Rader, C. (2020). Affinity maturation, humanization, and co-crystallization of a rabbit anti-human ROR2 monoclonal antibody for therapeutic applications. J Biol Chem 295, 5995-6006.
3. Li, M., Tao, Y., Shu, Y., LaRochelle, J. R., Steinauer, A., Thompson, D., Schepartz, A., Chen, Z. Y., and Liu, D. R. (2015). Discovery and characterization of a peptide that enhances endosomal escape of delivered proteins in vitro and in vivo. J Am Chem Soc 137, 14084-14093.
4. Nanna, A. R., Kel'in, A. V., Theile, C., Pierson, J. M., Voo, Z. X., Garg, A., Nair, J. K., Maier, M. A., Fitzgerald, K., and Rader, C. (2020). Generation and validation of structurally defined antibody-siRNA conjugates. Nucleic Acids Research 48, 5281-5293.
5. Nanna, A. R., Li, X., Walseng, E., Pedzisa, L., Goydel, R. S., Hymel, D., Burke Jr, T. R., Roush, W. R., and Rader, C. (2017). Harnessing a catalytic lysine residue for the one-step preparation of homogeneous antibody-drug conjugates. Nature Communications 8, 1112.
6. Nanna, A. R., and Rader, C. (2019). Engineering dual variable domains for the generation of site-specific antibody-drug conjugates. Methods Mol Biol 2033, 39-52.
7. Peng, H., Nerreter, T., Chang, J., Qi, J., Li, X., Karunadharma, P., Martinez, G. J., Fallahi, M., Soden, J., Freeth, J., et al. (2017). Mining naïve rabbit antibody repertoires by phage display for monoclonal antibodies of therapeutic utility. Journal of Molecular Biology 429, 2954-2973.
8. Qi, J., Hymel, D., Nelson, C. G., Burke, T. R., Jr., and Rader, C. (2019). Conventional and chemically programmed asymmetric bispecific antibodies targeting folate receptor 1. Front Immunol 10, 1994.
9. Vire, B., Skarzynski, M., Thomas, J. D., Nelson, C. G., David, A., Aue, G., Burke, T. R., Jr., Rader, C., and Wiestner, A. (2014). Harnessing the Fcμ receptor for potent and selective cytotoxic therapy of chronic lymphocytic leukemia. Cancer Res 74, 7510-7520.

All of the U.S. patents, U.S. patent application publications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A conjugate comprising:
a. a dual variable domain immunoglobulin molecule (Ig), or an antigen-binding fragment thereof, wherein the dual variable domain immunoglobulin molecule comprises:
i. a first variable domain that binds to a binding target; and
ii. a second variable domain that comprises a reactive residue;
b. a linker (L) covalently conjugated to the reactive residue of the second variable domain of the Ig; and
c. a double-stranded RNA (dsRNA) molecule conjugated to the linker, where the dsRNA is capable of inhibiting the expression of a target gene, where the dsRNA comprises a sense strand and an antisense strand, each strand having 14 to 40 nucleotides, wherein the antisense strand has sufficient complementarity to the target sequence to mediate RNA interference.

2. The conjugate of claim 1, wherein the dsRNA has a melting temperature (Tₘ) of from about 40° C. to about 80° C.

3. (canceled)

4. The conjugate according to claim 1, wherein the dsRNA comprises at least four 2′-fluoro modifications.

5. The conjugate of claim 1, wherein the dsRNA comprises a duplex region of 12-40 nucleotide base pairs in length.

6. The conjugate of claim 5, wherein the dsRNA comprises a duplex region of 18-25 nucleotide base pairs in length.

7. The conjugate of claim 1, wherein the dsRNA comprises a blunt end at 5′-end of the antisense strand.

8. The conjugate of claim 1, wherein the dsRNA comprises an overhang at 3′-end of the antisense strand.

9. (canceled)

10. The conjugate of claim 1, wherein the sense strand is covalently conjugated with the linker.

11.-14. (canceled)

15. The conjugate of claim 1, wherein the sense strand comprises 2 to 5 2′-fluoro modifications.

16.-18. (canceled)

19. The conjugate of claim 1, wherein the sense strand comprises 0 to 4 phosphorothioate internucleotide linkages.

20.-22. (canceled)

23. The conjugate of claim 1, wherein the antisense comprises 2 to 6 2′-fluoro modifications.

24.-26. (canceled)

27. The conjugate of claim 1, wherein the antisense comprises 2 to 4 phosphorothioate internucleotide linkages

28.-29. (canceled)

30. The conjugate of claim 1, wherein the antisense strand comprises at least one thermally destabilizing modification of the duplex within the first 9 nucleotide positions of the 5′ region.

31.-32. (canceled)

33. The conjugate of claim 1, wherein the antisense comprises a 5′-vinylphosphonate nucleotide at 5′-end.

34. The conjugate of claim 1, wherein the dsRNA comprises at least one 2′-OMe modification or the dsRNA comprises at least one locked nucleic acid (LNA) modification.

35.-38. (canceled)

39. The conjugate of claim 1, wherein the first variable domain of Ig is positioned closer to an N-terminus than the second variable domain.

40. The conjugate of claim 1, wherein Ig is a bispecific immunoglobulin molecule.

41.-42. (canceled)

43. The conjugate of claim 1, wherein:
(i) Ig comprises a chimeric immunoglobulin sequence; or
(ii) Ig comprises a humanized immunoglobulin sequence; or
(iii) Ig comprises a human immunoglobulin sequence.

44.-45. (canceled)

46. The conjugate of claim 1, wherein the binding target is a tumor cell surface antigen.

47. (canceled)

48. The conjugate of claim 1, wherein the linker L is a reversible linker, an irreversible linker, a cleavable linker, a non-cleavable linker, a branched linker, or a linear linker.

49.-53. (canceled)

54. The conjugate of claim 1, wherein the Ig further comprises a ligand.

55.-56. (canceled)

57. The conjugate of claim 1, wherein the second variable domain of Ig comprises an amino acid sequence selected from the group consisting of SEQ ID NO: 9, SEQ ID NO: 10, SEQ ID NO: 11, SEQ ID NO: 12, SEQ ID NO: 13, SEQ ID NO: 14, and any combinations thereof.

58. The conjugate of claim 1, wherein the Ig comprises a peptide linker between the first variable domain and the second variable domain.

59. (canceled)

60. The conjugate of claim 1, wherein the Ig comprises an amino acid sequence selected from the group consisting of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and any combinations thereof.

61. (canceled)

62. A pharmaceutical composition comprising the conjugate of claim 1 alone or in combination with a pharmaceutically acceptable carrier or excipient.

63. (canceled)

64. A method for silencing a target gene in a cell, the method comprising introducing a conjugate of claim 1 into the cell.

65. (canceled)


SEQUENCE LISTING (58 sequences)

SEQ ID NO: 1 - protein, 6 aa, Artificial Sequence
  Description of Artificial Sequence: Synthetic peptide
ASTKGP

SEQ ID NO: 2 - protein, 12 aa, Artificial Sequence
  Description of Artificial Sequence: Synthetic peptide
TVAAPSVFIFPP

SEQ ID NO: 3 - protein, 5 aa, Artificial Sequence
  Description of Artificial Sequence: Synthetic peptide
GGGGS

SEQ ID NO: 4 - protein, 10 aa, Artificial Sequence
  Description of Artificial Sequence: Synthetic peptide
GGGGSGGGGS

SEQ ID NO: 5 - protein, 15 aa, Artificial Sequence
  Description of Artificial Sequence: Synthetic peptide
GGGGSGGGGSGGGGS

SEQ ID NO: 6 - protein, 11 aa, Artificial Sequence
  Description of Artificial Sequence: Synthetic peptide
EPKSCDGGGGS

SEQ ID NO: 7 - protein, 16 aa, Artificial Sequence
  Description of Artificial Sequence: Synthetic peptide
EPKSCDGGGGSGGGGS

SEQ ID NO: 8 - protein, 21 aa, Artificial Sequence
  Description of Artificial Sequence: Synthetic peptide
EPKSCDGGGGSGGGGSGGGGS

SEQ ID NO: 9 - protein, 112 aa, Artificial Sequence
  Description of Artificial Sequence: Synthetic polypeptide
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIK

SEQ ID NO: 10 - protein, 118 aa, Artificial Sequence
  Description of Artificial Sequence: Synthetic polypeptide
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 11 - protein, 107 aa, Artificial Sequence
  Description of Artificial Sequence: Synthetic polypeptide
DVVMTQTPSSVPAAVGGTVTINCQASQSIDSNLAWFQQKPGQPPNLLIYDASTLASGVPSRFKGSAGKQFTLTISGVQREDAATYYCLGSYSRTEKAFGAGTKVEIK

SEQ ID NO: 12 - protein, 114 aa, Artificial Sequence
  Description of Artificial Sequence: Synthetic polypeptide
QEQLEESGGRLVTPGTPLTLTCTVSGFSLSNYHMSWVRQAPGKGLEWIGFITSGGSTYYASWAKGRFTISRTSTTVDLKITSPTTEDTATYFCARWNGYGGNMWGPGTLVTVSS

SEQ ID NO: 13 - protein, 108 aa, Artificial Sequence
  Description of Artificial Sequence: Synthetic polypeptide
DIQMTQSPSSLSASVGDRVTITCQASQSIDSNLAWYQQKPGKVPKLLIYDASTLASGVPSRFSGSGSGTDFTLTISSLQPEDVATYYCLGSYSRTEKAFGGGTKVEIK

SEQ ID NO: 14 - protein, 115 aa, Artificial Sequence
  Description of Artificial Sequence: Synthetic polypeptide
EVQLVESGGGLVQPGGSLRLSCAASGFTLSNYHMSWVRQAPGKGLEWVSFITSGGSTYYASWAKGRFTISRDNSKNTLYLQMNSLRAEDTAVYYCARWNGYGGNMWGQGTLVTVS

SEQ ID NO: 15 - protein, 219 aa, Artificial Sequence
  Description of Artificial Sequence: Synthetic polypeptide
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 448 aa, Artificial Sequence
  Description of Artificial Sequence: Synthetic polypeptide
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 17 - protein, 13 aa, Artificial Sequence
  Description of Artificial Sequence: Synthetic peptide
FSEAIKKIIDFLG

SEQ ID NO: 18 - protein, 356 aa, Artificial Sequence
  Description of Artificial Sequence: Synthetic polypeptide
MPMGSLQPLATLYLLGMLVASVLADIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 600 aa, Artificial Sequence
  Description of Artificial Sequence: Synthetic polypeptide
MPMGSLQPLATLYLLGMLVASVLAQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 20 - protein, 356 aa, Artificial Sequence
  Description of Artificial Sequence: Synthetic polypeptide
MPMGSLQPLATLYLLGMLVASVLADIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 599 aa, Artificial Sequence
  Description of Artificial Sequence: Synthetic polypeptide
MPMGSLQPLATLYLLGMLVASVLAQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 22 - protein, 356 aa, Artificial Sequence
  Description of Artificial Sequence: Synthetic polypeptide
MPMGSLQPLATLYLLGMLVASVLADIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 597 aa, Artificial Sequence
  Description of Artificial Sequence: Synthetic polypeptide
MPMGSLQPLATLYLLGMLVASVLAEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 24 - protein, 351 aa, Artificial Sequence
  Description of Artificial Sequence: Synthetic polypeptide
MDWTWRILFLVAAATGAHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 593 aa, Artificial Sequence
  Description of Artificial Sequence: Synthetic polypeptide
MDWTWRILFLVAAATGAHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 26 - protein, 365 aa, Artificial Sequence
  Description of Artificial Sequence: Synthetic polypeptide
MPMGSLQPLATLYLLGMLVADQQLTQSPSSLSASVGDRVTITCRASQSIGSWLSWYQQKPGKAPKLLIYGASNLASGVPSRFSGSRSGTDYTLTISSLQPEDFATYYCLGASPNGWAFGQGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECFSEAIKKIIDFLG

SEQ ID NO: 27 - protein, 591 aa, Artificial Sequence
  Description of Artificial Sequence: Synthetic polypeptide
MPMGSLQPLATLYLLGMLVAEQQVVESGGGLVQPGGSLRLSCAVSGFSLNSYGVIWVRQAPGKGLEYVSIIGSSGNTYYASSVKGRFTISRDTRLNTVYLQMNSLRAEDTAVYFCARYYGDSGFDSWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 28 - protein, 486 aa, Artificial Sequence
  Description of Artificial Sequence: Synthetic polypeptide
MDWTWRILFLVAAATGAHSDVVMTQTPSSVPAAVGGTVTINCQASQSIDSNLAWFQQKPGQPPNLLIYDASTLASGVPSRFKGSGAGKQFTLTISGVQREDAATYYCLGSYSRTEKAFGAGTKVEIKGGGGSGGGGSGGGGSQEQLEESGGRLVTPGTPLTLTCTVSGFSLSNYHMSWVRQAPGKGLEWIGFITSGGSTYYASWAKGRFTISRTSTTVDLKITSPTTEDTATYFCARWNGYGGNMWGPGTLVTVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGA

SEQ ID NO: 29 - protein, 699 aa, Artificial Sequence
  Description of Artificial Sequence: Synthetic polypeptide
MDWTWRILFLVAAATGAHSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 30 - protein, 238 aa, Artificial Sequence
  Description of Artificial Sequence: Synthetic polypeptide
MDWTWRILFLVAAATGAHSELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31-35 - skipped (5 entries)

SEQ ID NO: 36 - RNA, 21 nt, Artificial Sequence
  Description of Artificial Sequence: Synthetic oligonucleotide
uacuguuggauugauucgaaa

SEQ ID NO: 37 - RNA, 21 nt, Artificial Sequence
  Description of Artificial Sequence: Synthetic oligonucleotide
uacuguuggauugauucgaaa

SEQ ID NO: 38 - RNA, 21 nt, Artificial Sequence
  Description of Artificial Sequence: Synthetic oligonucleotide
ugggauuucauguaaccaaga

SEQ ID NO: 39 - RNA, 21 nt, Artificial Sequence
  Description of Artificial Sequence: Synthetic oligonucleotide
uacuguuggauugauucgaaa

SEQ ID NO: 40 - RNA, 21 nt, Artificial Sequence
  Description of Artificial Sequence: Synthetic oligonucleotide
uacuguuggauugauucgaaa

SEQ ID NO: 41 - RNA, 21 nt, Artificial Sequence
  Description of Artificial Sequence: Synthetic oligonucleotide
ugggauuucauguaaccaaga

SEQ ID NO: 42 - DNA, 20 bp, Artificial Sequence
  Description of Artificial Sequence: Synthetic primer
cctgtacgccaacacagtgc

SEQ ID NO: 43 - DNA, 20 bp, Artificial Sequence
  Description of Artificial Sequence: Synthetic primer
atactcctgcttgctgatcc

SEQ ID NO: 44 - DNA, 19 bp, Artificial Sequence
  Description of Artificial Sequence: Synthetic primer
aaaatggcagtgcgtttag

SEQ ID NO: 45 - DNA, 20 bp, Artificial Sequence
  Description of Artificial Sequence: Synthetic primer
tttgaaggcagtctgtcgta

SEQ ID NO: 46 - RNA, 21 nt, Artificial Sequence
  Description of Artificial Sequence: Synthetic oligonucleotide
uacuguuggauugauucgaaa

SEQ ID NO: 47 - RNA, 21 nt, Artificial Sequence
  Description of Artificial Sequence: Synthetic oligonucleotide
uacuguuggauugauucgaaa

SEQ ID NO: 48 - RNA, 21 nt, Artificial Sequence
  Description of Artificial Sequence: Synthetic oligonucleotide
uacuguuggauugauucgaaa

SEQ ID NO: 49 - DNA, 19 bp, Artificial Sequence
  Description of Artificial Sequence: Synthetic primer
tcaccactgccagctgcta

SEQ ID NO: 50 - DNA, 19 bp, Artificial Sequence
  Description of Artificial Sequence: Synthetic primer
aaactccggatggcctcat

SEQ ID NO: 51 - RNA, 21 nt, Artificial Sequence
  Description of Artificial Sequence: Synthetic oligonucleotide
uacuguuggauugauucgaaa

SEQ ID NO: 52 - RNA, 21 nt, Artificial Sequence
  Description of Artificial Sequence: Synthetic oligonucleotide
ugagugguuuucuuuuuccua

SEQ ID NO: 53 - RNA, 23 nt, Artificial Sequence
  Description of Artificial Sequence: Synthetic oligonucleotide
uaggaaaaagaaaaccacucauc

SEQ ID NO: 54 - DNA, 23 bp, Artificial Sequence
  Description of Artificial Sequence: Synthetic oligonucleotide
  Description of Combined DNA/RNA Molecule: Synthetic oligonucleotide
tuucgaaucaauccaacaguagc

SEQ ID NO: 55 - DNA, 23 bp, Artificial Sequence
  Description of Artificial Sequence: Synthetic oligonucleotide
  Description of Combined DNA/RNA Molecule: Synthetic oligonucleotide
tcuugguuacaugaaaucccauc

SEQ ID NO: 56 - RNA, 21 nt, Artificial Sequence
  Description of Artificial Sequence: Synthetic oligonucleotide
uacuguuggauugauucgaaa

SEQ ID NO: 57 - protein, 60 aa, Artificial Sequence
  Description of Artificial Sequence: Synthetic polypeptide
  MISC_FEATURE (1)..(60): This sequence may encompass 1-10 “(Gly)n Ser” repeating units where n=2-5
GGGGGSGGGGGSGGGGGSGGGGGSGGGGGSGGGGGSGGGGGSGGGGGSGGGGGSGGGGGS

SEQ ID NO: 58 - protein, 13 aa, Artificial Sequence
  Description of Artificial Sequence: Synthetic peptide
GLFDIIKKIAESF